SEQUENCE LISTING (44 sequences)

SEQ ID NO: 1 - DNA, 21 bp, Artificial Sequence
  synthetic oligonucleotide
gtggactcttgaaagtactat

SEQ ID NO: 2 - DNA, 21 bp, Artificial Sequence
  synthetic oligonucleotide
ccacaagtcaaacctttattt

SEQ ID NO: 3 - DNA, 20 bp, Artificial Sequence
  synthetic oligonucleotide
cctgagaggtcattcggtca

SEQ ID NO: 4 - DNA, 20 bp, Mus musculus
gaccttccaggatgaggaca

SEQ ID NO: 5 - DNA, 20 bp, Mus Musculus
tccattgaggtggagagctt

SEQ ID NO: 6 - DNA, 22 bp, Mus Musculus
ccatagctacctggagtacatg

SEQ ID NO: 7 - DNA, 22 bp, Mus Musculus
tggaaattggggtaggaaggac

SEQ ID NO: 8 - DNA, 21 bp, Mus musculus
tcttctcattcctgcttgtgg

SEQ ID NO: 9 - DNA, 20 bp, Mus musculus
cacttggtggtttgctacga

SEQ ID NO: 10 - DNA, 18 bp, Mus musculus
tctgatgcagcaggtggg

SEQ ID NO: 11 - DNA, 24 bp, Mus musculus
agggctctccagacttctgctctg

SEQ ID NO: 12 - DNA, 18 bp, Mus musculus
gggggagagtcgatccag

SEQ ID NO: 13 - DNA, 20 bp, Mus musculus
ccccagcatcttcaccttta

SEQ ID NO: 14 - DNA, 20 bp, Mus musculus
gctctgctgaaaacccagag

SEQ ID NO: 15 - DNA, 20 bp, Mus musculus
cccaatgggttcttgatgtc

SEQ ID NO: 16 - DNA, 20 bp, Mus musculus
caccttcggtatggcaactt

SEQ ID NO: 17 - DNA, 20 bp, Mus musculus
gcaaggcctcagaatcagac

SEQ ID NO: 18 - DNA, 18 bp, Mus musculus
gggggagagtcgatccag

SEQ ID NO: 19 - DNA, 20 bp, Mus musculus
ccccagcatcttcaccttta

SEQ ID NO: 20 - DNA, 20 bp, Mus musculus
ctccgaggtctacgcaaatc

SEQ ID NO: 21 - DNA, 20 bp, Mus musculus
cagctccagggcatagagag

SEQ ID NO: 22 - DNA, 19 bp, Mus musculus
gacattgccaccacagagg

SEQ ID NO: 23 - DNA, 20 bp, Mus musculus
ctgctcttggatgtcctgct

SEQ ID NO: 24 - DNA, 21 bp, Homo sapiens
ggctccaggcggtgcttgttc

SEQ ID NO: 25 - DNA, 20 bp, Homo sapiens
agacggcgatgcggctgatg

SEQ ID NO: 26 - DNA, 22 bp, Homo sapiens
cactggcagaaaacaacctgaa

SEQ ID NO: 27 - DNA, 22 bp, Homo sapiens
accaggcaagtctcctcattga

SEQ ID NO: 28 - DNA, 20 bp, Homo sapiens
ttgatggcaaccagttccag

SEQ ID NO: 29 - DNA, 20 bp, Homo sapiens
tcatcccaagcagcagatga

SEQ ID NO: 30 - DNA, 20 bp, Homo sapiens
tgttcccaagcagcagatga

SEQ ID NO: 31 - DNA, 21 bp, Homo sapiens
cgcagatcacccagaagatcg

SEQ ID NO: 32 - DNA, 20 bp, Homo sapiens
ttcgtcgcatttgtccacca

SEQ ID NO: 33 - DNA, 20 bp, Mus musculus
ttttccaggcaaagcagatt

SEQ ID NO: 34 - DNA, 20 bp, Mus musculus
atgtatgggcacaagcacaa

SEQ ID NO: 35 - DNA, 20 bp, Mus musculus
taatccttgggagtggagca

SEQ ID NO: 36 - DNA, 20 bp, Mus musculus
gtgtggctggagtctggagt

SEQ ID NO: 37 - DNA, 20 bp, Mus musculus
aataatgcccatttccacca

SEQ ID NO: 38 - DNA, 20 bp, Mus musculus
gctgtgaaattttcccttgg

SEQ ID NO: 39 - DNA, 20 bp, Mus musculus
ctgtcctggcttaggctttg

SEQ ID NO: 40 - DNA, 20 bp, Mus musculus
ggaaaccccacgactgacta

SEQ ID NO: 41 - DNA, 20 bp, Mus musculus
ccggctgctgaggagtattt

SEQ ID NO: 42 - DNA, 20 bp, Mus musculus
agcaatgccctcggtttaca

SEQ ID NO: 43 - DNA, 20 bp, Mus musculus
cgcggatcatgctttctgtg

SEQ ID NO: 44 - DNA, 20 bp, Mus musculus
gaaaagcaagcagccaacca

(19) United States
(12) Patent Application Publication
(10) Pub. No.: US 2023/0310421 A1
(43) Pub. Date: Oct. 5, 2023

(54) MOLECULES FOR USE IN THE TREATMENT OF VIRAL INFECTIONS

(71) Applicants: ISTITUTO EUROPEO DI ONCOLOGIA S.R.L., Milano (MI) (IT); UNIVERSITA' DEGLI STUDI DI MILANO, Milano (MI) (IT)

(72) Inventors: Piergiuseppe PELICCI, Milano (MI) (IT); Saverio MINUCCI, Opera (MI) (IT); Fabio SANTORO, Milano (MI) (IT); Mauro ROMANENGHI, Milano (MI) (IT); Luca MAZZARELLA, Milano (MI) (IT); Paul-Edward MASSA, Milano (MI) (IT); Bruno ACHUTTI DUSO, Milano (MI) (IT)

(73) Assignees: ISTITUTO EUROPEO DI ONCOLOGIA S.R.L., Milano (MI) (IT); UNIVERSITA' DEGLI STUDI DI MILANO, Milano (MI) (IT)

(21) Appl. No.: 17/996,113

(22) PCT Filed: Apr. 14, 2021
(86) PCT No.: PCT/EP2021/059707

§ 371 Date: Oct. 13, 2022

(30) Foreign Application Priority Data
Apr. 14, 2020 (IT) 102020000007873

(51) Int. Cl. A61K 31/4965; A61K 45/06; A61K 31/4409; A61K 31/4245; A61P 31/14

(52) U.S. Cl. CPC A61K 31/4965; A61K 45/06; A61K 31/4409; A61K 31/4245; A61P 31/14

(57) ABSTRACT

The present invention relates to LSD1 inhibitors for use in the treatment and/or prevention of a viral infection caused by and/or associated with RNA viruses, preferably Coronaviridae. The LSD1 inhibitors are able to inhibit or prevent the viral induced increased expression of inflammatory cytokines while sparing the expression of Interferon and Interferon-Stimulated Genes. The present invention further concerns a combination and a pharmaceutical composition including the molecules or combinations thereof.

### TECHNICAL FIELD

The present invention relates to LSD1 inhibitors for use in the treatment and/or prevention of a viral infection and/or viral disease caused by and/or associated with RNA viruses, preferably Coronaviridae. The LSD1 inhibitors are able to inhibit or prevent the viral induced increased expression of inflammatory cytokines while sparing the expression of Interferon and Interferon-Stimulated Genes. The present invention further concerns a combination and a pharmaceutical composition comprising said molecule or combinations thereof.

### BACKGROUND ART

The ongoing Covid-19 pandemics is unquestionably a major event in human history. Its dramatic impact is favored by several elements of novelty including the relatively recent human-virus coevolution (Cui et al., 2019), the elevated degree of connectivity in modern society (Haw et al., 2019) and the elevated numbers of infections and deaths, as compared to the other two coronavirus epidemics of this millennium, severe acute respiratory syndrome (SARS) and Middle East respiratory syndrome (MERS) (Wang et al., 2020).

Despite recent trials with antiviral combinations, mortality for hospitalized patients remains elevated, up to 25% (Cao et al., 2020). An alternative strategy is the treatment of early-stage disease, which might reduce hospitalization, thus relieving the pressure on the currently overwhelmed healthcare systems, and minimize the risk of hospital-based viral spread, a major route of infection in this pandemics (Wang et al., 2020). To this end, it is necessary to adopt novel approaches, including novel modalities of treatment, by targeting biological processes of the early phases of viral spread.

Increasing evidence suggests that the severity of COVID-19 pathology is dictated by the profile of immune mediators produced in response to SARS-CoV2 infection. High levels of inflammatory cytokines typical of the innate immune response, such as IL6, TNF and ILL are associated with severe disease, whereas expression of molecules with direct antiviral activity, such as type I interferon and its downstream targets (Interferon-Stimulated Genes, ISGs) are associated with better outcome [Del Valle D M et al, 2020; Hadjadj J et al, 2020; Bastard P et al, 2020; 370]. Key transcriptional regulators of these innate responses to pathogens include Nuclear Factor-Kappa B (NFKB), which regulates transcription of proinflammatory cytokines, and the family of Interferon-Regulated Factors (IRFs), which promote expression of Interferons and ISGs [Fung T S et al, 2019]. These responses are structured in self-amplifying paracrine loops where downstream targets (e.g. IL1b and TNF for NFKB; type I Interferons for IRFs) are themselves activators of the same pathway, a logic architecture that allows the signal to propagate in space and time to rapidly arrest infection spread in the involved organ [Hayden M S et al, 2006; Hall J C et al 2010]. Unbridled loop activation, however, can lead to extensive local or systemic damage, further amplified by the recruitment of additional effector cells like neutrophils or lymphocytes. The initiation and duration of the innate transcriptional responses is further regulated by multiple cofactors, among which chromatin-modifying enzymes are thought to play a key role [Zhou H et al 2010]. The Lysine Demethylase 1 (LSD1; also known as KDM1A), in particular, has been implicated in both NFKB and IRF pathways and is pharmacologically targetable. Multiple LSD1 inhibitors have completed initial phases of clinical development for oncological indications with acceptable toxicity profiles and may be amenable to repurposing. In a mouse system of Lipopolysaccharide (LPS)-mediated NFKB activation using bone marrow-derived macrophages (BMDM), genetic ablation of Lsd1 was found to be associated with decreased nuclear translocation of NFKB and reduced binding to its target promoters, resulting in decreased transcription of proinflammatory cytokines and improved survival in a sepsis mouse model [Kim D et al, 2018]. On the other hand, LSD1 has also been implicated in suppressing endogenous retroviral elements (ERVs) in mouse melanoma cells. Loss of LSD1 induced up-regulation of ERV expression with consequent accumulation of double-stranded RNA (dsRNA), recognition by the dsRNA-sensing pathway MDA5-IRF3 and activation of an interferon-mediated response [Sheng W et al 2018].

Transcription factor dynamics in human and murine coronavirus infections have been mostly assessed using epithelial or fibroblast cell lines [Canton J et al 2018; Zhou H et al, 2007]. Although epithelia and connective tissue are sites of active involvement in the pathogenesis of coronavirus infections, increasing evidence points to a central role for cells of the monocyte-macrophage compartment, which are strongly modulated during Covid19 in relation to disease severity [Merad M et al, 2020; Schulte-Schrepping J et al, 2020; Zhou Z et al, 2020]. These cells are equipped with a vast array of pathogen-sensing mechanisms and play a key role in the generation and amplification of inter-cellular signaling loops in innate immunity [Bagnall J, et al, 2018; Lawrence T et al, 2011; Lech M et al, 2010]. Notably, alveolar macrophages from Covid19 patients or SARS-CoV2-infected African Green Monkeys contain viral RNA in significant amounts, including negative strand sequences that are indicative of active replication. However, the study of the molecular mechanisms involved in macrophage responses to human SARS-CoV2 has been hampered by the lack of in vitro models of productive infection [Dalskov L et at, 2020], as also observed for SARS-CoV1 [Yilla M et al, 2005; Tseng C-T K et al, 2005] suggesting that virus uptake by macrophages is mediated by elements of the in vivo environment that are missing in vitro. In vivo, non-receptor-mediated entry mechanisms have been suggested, such as antibody-mediated endocytosis or phagocytosis of infected cells [Lee W S et al, 2020] The dynamics of NFKB and IRF activation in macrophage coronavirus infections and the potential role of LSD1 have not been extensively characterized to date. As model system, we used the Murine Hepatitis Virus (MHV), strain A59, a beta-coronavirus phylogenetically close to human SARS-CoV1/2 [Fung T S et al, 2019] and able to produce a disease highly similar to that triggered by human SARS-CoV [Qing H et al, 2020; Yang Z et al, 2014]. MHV entry is mediated by the murine CEACAM1 receptor, which is highly expressed in mouse macrophages, both in vivo and in vitro [Hirai A et al, 2010], thus allowing to circumvent the intrinsic difficulties to model macrophage infection in vitro by SARS-CoVs. To investigate cell-intrinsic responses and paracrine signaling, macrophages were studied alone or in cocultures with fibroblasts (L929), an established model for the study of coronavirus innate responses [Zhou H et al, 2007]. In the course of the present invention it has been shown that murine macrophages activate an NFKB and IRF-dependent transcriptional response that is highly similar to that elicited in human SARS-CoV2-infected alveolar macrophages, defining a tractable system to investigate the signaling pathways activated upon coronavirus infection. The dynamics of the NFKB/IRF activation has been characterized, key roles for IRF1 and LSD1 have been identified and the molecular rationale for investigating LSD1 inhibitors in the treatment of Coronavirus disease was provided. The potential role of LSD1 inhibitor in the treatment of viral infections has been previously suggested for several viruses, including Herpesviridae and Orthomyxoviridae, and patent applications have been submitted for these uses (Future Med. Chem. (2018) 10(10), 1133-1136; Nature Medicine volume 15, pages 1312-1317(2009)). However, all viruses for which this therapeutic strategy has been assessed are DNA-based viruses which require genomic integration as a necessary step of their infectious cycle, and the proposed mechanism of action for LSD1 inhibition concerns its impact on the chromatin landscape of the viral integration site. No evidence exists so far on an impact of LSD1 inhibition on the host innate response to coronavirus infections.

Finally, currently approved therapies for the treatment of coronavirus infections are very limited and have significant limitations; in particular, steroids, the only approved class of drugs acting on the host immune response, is effective in reducing the intensity of the inflammatory response but has well known suppressive activity on the interferon response, which is considered essential for effective clearance of coronaviruses. Thus, steroids appear ineffective or even detrimental in some clinical settings, with evidence of potentially prolonged clearance times and higher incidence of bacterial superinfections (J Med Virol. 2021 Apr. 6. doi: 10.1002/jmv.27000; Crit Care 24, 696 (2020). https://doi.org/10.1186/s13054-020-03400-9). Therapies able to decrease the inflammatory response without affecting the interferon response may positively impact treatment of coronavirus infections.

### SUMMARY OF THE INVENTION

The present invention starts from the evidence that macrophages are central to Covid19 pathology but difficult to investigate in humans as they cannot be productively infected in vitro. Inventors show that transcriptional responses of mouse macrophages to the homologous MHV-A59 are highly reminiscent of those observed in human macrophages infected in vivo by SARS-CoV2, and include simultaneous activation of two major programs: i) an NFKB-dependent module of inflammatory cytokines, cytotoxic to neighboring cells regardless of infection; ii) a type I-interferon/Interferon-Stimulated Genes (ISG) module which limits viral spread without cytotoxicity. Inventors identified Interferon-Related Factor 1 (IRF1) as the master regulator of the antiviral module, while other IRF family members conventionally implicated in antiviral responses were not involved. Ablating the lysine demethylase LSD1 selectively suppressed NFKB while sparing IRF1 (unlike steroids that suppressed both modules indiscriminately), activated ISGs in an interferon-independent manner and blocked viral egress through the lysosomal pathway, providing a rationale for repurposing LSD1 inhibitors for treatment and/or prevention of viral infection or viral disease caused by and/or associated with RNA viruses, preferably Coronaviridae, more preferably SARS COV-2.

### DETAILED DESCRIPTION OF THE INVENTION

It is therefore an object of the invention an LSD1 inhibitor for use in the treatment and/or prevention of a viral infection and/or viral disease caused by and/or associated with RNA viruses, preferably Coronaviridae.

The LSD1 inhibitor as defined above is selected from:
a) a compound of general formula (I)

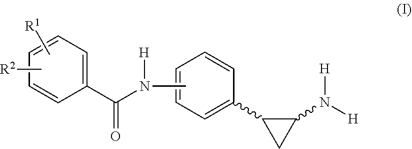

or a pharmaceutically acceptable salt or solvate thereof, wherein
R¹ is 4-methylpiperazin-1-yl, 1-methylpiperidin-4-yl, oxooxazolidin-3-yl, piperidin-1-yl or morpholin-4-yl; and
R² is hydrogen, halogen, C₁₋₆ alkyl, C₁₋₆ alkoxy, C₁₋₆ haloalkyl, C₁₋₆ haloalkoxy, or benzyloxycarbonylamino;
and/or
b) anyone of the following compounds:
ORY-1001 (or iadademstat), CC-90011, ORY-2001 (or vafidemstat), GSK-2879552, IMG-7289 (or bomedemstat), INCB059872, 4SC-202 (or domatinostat), Seclidemstat, TAK-418, SYHA-1807, BEA-17, HM-97211, HM-97346, JBI-097, JBI-128, ORY-3001, RN-1, SP-2509, T-3775440, T-448, EPI-110; and pharmaceutically acceptable salt or solvate thereof. The LSD1 inhibitor is also preferably any one or more of the compounds disclosed in WO2011131576, WO2014086790, WO2015181380, WO2016034946, WO2017198780 or WO2019034774, all of which are herein enclosed by reference.

Preferably in the LSD1 inhibitor of general formula (I) as defined above:
R¹ is 4-methylpiperazin-1-yl and R² is hydrogen; or
R¹ is 4-methylpiperazin-1-yl and R² is benzyloxycarbonylamino; or
R¹ is 1-methylpiperidin-4-yl and R² is hydrogen; or
R¹ is 1-methylpiperidin-4-yl and R² is benzyloxycarbonylamino; or
R¹ is 2-oxooxazolidin-3-yl and R² is hydrogen; or
R¹ is 2-oxooxazolidin-3-yl and R² is benzyloxycarbonylamino;
R¹ is piperidin-1-yl and R² is hydrogen; or
R¹ is piperidin-1-yl and R² is benzyloxycarbonylamino; or
R¹ is morpholin-4-yl and R² is hydrogen; or
   R¹ is morpholin-4-yl and R² is benzyloxycarbonylamino; and pharmaceutically acceptable salt or solvate thereof.

Still preferably the LSD1 inhibitor of general formula (I) is selected from:
N-[4-[(1S,2R)-2-aminocyclopropyl]phenyl]-4-(4-methylpiperazin-1-yl)benzamide;
N-[4-[(1S,2R)-2-aminocyclopropyl]phenyl]-3-(2-oxooxazolidin-3-yl)benzamide;
N-[4-[(1R,2S)-2-aminocyclopropyl]phenyl]-4-(4-methylpiperazin-1-yl)benzamide;
N-[4-[(1R,2S)-2-aminocyclopropyl]phenyl]-3-(2-oxooxazolidin-3-yl)benzamide;
N-[4-[trans-2-aminocyclopropyl]phenyl]-4-(4-methylpiperazin-1-yl)benzamide;
N-[4-[trans-2-aminocyclopropyl]phenyl]-4-(1-methylpiperidin-4-yl)benzamide;
N-[4-[trans-2-aminocyclopropyl]phenyl]-3-(2-oxooxazolidin-3-yl)benzamide;
N-[4-[trans-2-aminocyclopropyl]phenyl]-4-(2-oxooxazolidin-3-yl)benzamide;
Benzyl N-[5-[[4-(trans-2-aminocyclopropyl)phenyl]carbamoyl]-2-(4-methylpiperazin-1-yl)phenyl]carbamate;
Benzyl N-[4-[[4-(trans-2-aminocyclopropyl)phenyl]carbamoyl]-2-(4-methylpiperazin-1-yl)phenyl]carbamate;
Benzyl N-[5-[[4-[trans-2-aminocyclopropyl]phenyl]carbamoyl]-2-(1-piperidyl)phenyl]carbamate;
Benzyl N-[5-[[4-[trans-2-aminocyclopropyl]phenyl]carbamoyl]-2-morpholinophenyl]carbamate
   and pharmaceutically acceptable salt or solvate thereof.

Even more preferably the LSD1 inhibitor is selected from the group consisting of: N-[4-[(1S,2R)-2-aminocyclopropyl]phenyl]-4-(4-methylpiperazin-1-yl)benzamide (DDP38003), N-[4-[trans-2-aminocyclopropyl]phenyl]-4-(4-methylpiperazin-1-yl)benzamide, N-[4-[(1R,2S)-2-aminocyclopropyl]phenyl]-4-(4-methylpiperazin-1-yl)benzamide (DDP37368), ORY-1001 (or iadademstat), ORY-2001 (or vafidemstat) and a pharmaceutically acceptable salt or solvate thereof.

It is a further object of the invention the LSD1 inhibitor as defined above for the treatment and/or prevention of the viral induced increased expression of inflammatory cytokines while sparing the expression of Interferon and Interferon-Stimulated Genes. Inflammatory cytokines include but are not limited to Ccl3, Ccl4, Ccl5, Ccl8, Cxcl2, Cxcl3, Il10, Il12a, Il12b, Il1a, Il1b, Il6, TNFa and their orthologs in different species. The Interferon and Interferon-Stimulated genes include but are not limited to Ifit1, Ifit2, Ifit3, Ifitm3, Isg15, Mx1, Mx2, Oas1, Oas2, Oas3, MDA5, RIG-I, Irf1, Irf2, Irf7 and genes encoding for type I, II and III interferon and their orthologs in different species.

It is also an object of the present invention any combination of two or more molecules as defined above. In particular, it is a further object of the present invention a combination for use in the treatment and/or prevention of viral infection caused by and/or associated with RNA viruses, preferably Coronaviridae, comprising at least one LSD1 inhibitor, or the pharmaceutically acceptable salt, hydrate or solvate thereof as defined above and at least one other therapeutic agent. Preferably, said one or more therapeutic agents are selected from the group consisting of: antiviral drugs, cytidine-deaminase inhibitors, retinoic acid, lipoic acid, anti-coagulating agents, Vitamin D, antibiotics, corticosteroids, curcumin, procaine, hydralazine, epigallocatechin gallate, RG-108, 3-nitro-2-(3-nitrophenyl) flavone, disulfiram, isoxazoline. More preferably, said one or more therapeutic agents are selected from the group consisting of Interferon (in particular interferon alpha), Ribavirin, Lopinavir/Ritonavir, chloroquine, hydroxychloroquine, heparin, cedazuridine.

It is also an object of the present invention a pharmaceutical composition for use in the treatment and/or prevention a viral infection and/or viral disease caused by and/or associated with RNA viruses, preferably Coronaviridae, comprising:
a) the LSD1 inhibitor, or the pharmaceutically acceptable salt or solvate as defined above or the combination as defined above; and
b) at least one pharmaceutically acceptable excipient and/or carrier.

Preferably the viral infection or disease is an infection or disease of the respiratory tract. Preferably the viral infection or disease is an infection of the gastrointestinal tract, still preferably of the kidneys, still preferably of the central nervous system. Preferably, the viral infection or disease is caused by and/or associated with RNA viruses. Preferably, said RNA viruses are of the Coronaviridae family. More preferably the virus is selected from the group consisting of: human coronavirus 229E; human coronavirus 0C43; Severe Acute Respiratory Syndrome coronavirus (SARS-CoV); human Coronavirus NL63 (HCoV-NL63, New Haven coronavirus); human coronavirus HKU1; Middle East respiratory syndrome coronavirus (MERS-CoV); and Severe Acute Respiratory Syndrome coronavirus 2 (SARS-CoV-2).

Preferably, the viral infection is a coronavirus infection selected from the group consisting of: COVID-19, SARS, MERS, any other severe acute respiratory syndrome caused by Coronavirus, upper respiratory tract infections, pneumonia, pneumonitis, bronchitis.

The LSD1 inhibitors of the invention can be used in combination or in association with a further therapeutic agent, preferably an epigenetic regulator. Said epigenetic regulator is preferably selected from DNMT inhibitors, HDAC inhibitors, histone methyltransferase (HMT) inhibitors such as EZH1/2 inhibitors or PRMT inhibitors, dual HDAC/LSD1 inhibitors, BET inhibitors or a pharmaceutically acceptable salt, hydrate or solvate thereof and/or combinations thereof.

Preferably, the DNMT inhibitor is selected from the group consisting of: 5-azacytidine (SAC, trade name; Vidaza®, Azadine), 5-aza-2′-deoxycytidine (5-aza-CdR; DAC; also known as Decitabine, trade name; Dacogen®), CC-486 (oral Vidaza), 4′-thio-2′-deoxycytidine (or TdCyd), 5aza-4′-thio-2′-deoxycytidine (or aza-T-dCyd), Guadecitabine sodium (SGI-110), Zebularine, CP-4200, Flucytosine, Roducitabine, NSC-764276, EF-009, KM-101, NTX-301, Sinefungin, an antisense oligonucleotide such as MG-98, or a pharmaceutically acceptable salt, hydrate or solvate thereof and/or combinations thereof.

Preferably, the HDAC inhibitor is a pan-HDAC inhibitor or is HDAC1-3 selective or is HDAC6 selective. Preferably, the HDAC inhibitor is selected from the group consisting of: Vorinostat (trade name; ZOLINZA®), Romidepsin (trade name; Istodax®), Panobinostat (trade name; FARYDAK®), Belinostat, Entinostat, Dacinostat, Domatinostat, Resminostat, Valproic acid, Valproate sodium, Quisinostat hydrochloride, CUDC-101, Tefinostat, Givinostat, Mocetinostat, Chidamide, Abexinostat, Pracinostat, Tacedinaline, Butyric acid, Pivanex, 4-phenylbutyric acid (or sodium phenylbutyrate), Tucidinostat (or chidamide, trade name; Epidaza®), Nanatinostat, Fimepinostat, Remetinostat, Ricolinostat, Tinostamustine, Pracinostat, APH-0812, CG-745, CKD-506, CKD-581, CXD-101, FX-322, YPL-001, CFH-367C, Citarinostat, CKD-504, CUDC-908, HG-146, KA-2507, Lipocurc, MPT-0E028, NBM-BMX, OBP-801, OKI-179, RDN-929, VTR-297, REC-2282, CS-3003, ACY-1035, ACY-1071, ACY-1083, ACY-738, ACY-775, ACY-957, ADV-300, AN-446, AP-001 (or Metavert), Arginine Butyrate, BMN-290, C-1A, CG-1521, CKD-509, CKD-L, CM-414, Crocetin, CS-3158, CT-101, CX-1026, RCY-1410, SE-7552, SKLB-23bb, CY-190602, JBI-097, JBI-128, JMF-3086, KAN-0440262, KDAC-0001, Largazole, MPT-0B291, MPT-0G211, MRx-0029, MRX-0573, MRX-1299, NHC-51, Nexturastat A, OKI-422, QTX-125, RCY-1305, SP-1161, SP-259, SRX-3636, ST-7612AA1, TJC-0545, Trichosic, YH-508, HSB-501, NBM-1001, QTX-153, RDN-1201, ROD-119, ROD-1246, ROD-1275, ROD-1702, ROD-2003, ROD-2089, ROD-702 and RTSV-5, or a pharmaceutically acceptable salt, hydrate or solvate thereof and/or combinations thereof.

Preferably, the HMT inhibitor is:
i. an EZH1/2 inhibitor selected from the group consisting of: Tazemetostat hydrobromide (trade name; TAZVERIK™), Valemetostat, ZLD1039, GSK926, GSK126, PF-06821497, UNC1999, CPI-1205, MC-3629, CPI-0209, SHR-2554, CPI-169, EBI-2554, GSK-343, HM-97594, IONISEZH-22.5Rx, JQEZ-5, MS-1943, ORS-1, TBL-0404, KM-301, GSK2816126 or a pharmaceutically acceptable salt, hydrate or solvate thereof and/or combinations thereof, and/or
ii. a PRMT inhibitor, such as a PRMT5 inhibitor or a PRMT1 inhibitor,
preferably said PRMT5 inhibitor is selected from the group consisting of: GSK3326595, JNJ-64619178, PF-06939999, PRT-543, PRT-811, JBI-778, GSK-3235025 or a pharmaceutically acceptable salt, hydrate or solvate thereof and/or combinations thereof,
preferably said PRMT1 inhibitor is GSK3368715 or a pharmaceutically acceptable salt, hydrate or solvate thereof and/or combinations thereof,
and/or
iii. a DOT1L inhibitor, preferably Pinometostat (or EPZ-5676)
or a pharmaceutically acceptable salt, hydrate or solvate thereof and/or combinations thereof, and/or
iv. APTA-16
or a pharmaceutically acceptable salt, hydrate or solvate thereof, and/or
v. a Histone Lysine N Methyltransferase (EHMT2 or G9a) inhibitor, preferably selected from the group consisting of: BIX-01294, a BIX-01294 analogue (or TM-2115)
or a pharmaceutically acceptable salt, hydrate or solvate thereof and/or combinations thereof, and/or
vi. a dual inhibitor against G9a and DNMTs, preferably CM-272
or a pharmaceutically acceptable salt, hydrate or solvate thereof and/or combinations thereof, and/or
vii. a menin-MLL1 (or KMT2A) interaction inhibitor, preferably MI-3454 and/or KO-539, or a pharmaceutically acceptable salt, hydrate or solvate thereof and/or combinations thereof,
viii. a SETD2 inhibitor, preferably EPZ-040414
or a pharmaceutically acceptable salt, hydrate or solvate thereof and/or combinations thereof. Preferably, the dual HDAC/LSD1 inhibitor is selected from the group consisting of: the molecules described in WO 2017/195216; D. Sivanandhan et al., “Abstract 3509: Novel dual inhibitors of LSD1-HDAC for treatment of cancer”, Cancer Research, 2015, 75, 15, doi: 10.1158/1538-7445.AM2015-3509; D. Sivanandhan et al., “Abstract 5860: Novel, first-in-class dual inhibitors of lysine specific demethylase 1 (LSD1) and histone deacetylatse 1 (HDAC) for treatment of cancer”, Cancer Research, 2018, 78, 13, doi: 10.1158/1538-7445.AM2018-5860; D. Sivanandhan et al., “Abstract 1382: Novel dual inhibitors of LSD1-HDAC6/8 for treatment of cancer”, Cancer Research, 2018, 78, 13, doi: 10.1158/1538-7445.AM2018-1382; Kalin J H, Wu M, Gomez A V, et al.; “Targeting the CoREST complex with dual histone deacetylase and demethylase inhibitors.”; Nat. Commun. 2018; 9(1):53. doi:10.1038/s41467-017-02242-4, all of which are herein enclosed by reference, or a pharmaceutically acceptable salt, hydrate or solvate thereof and/or combinations thereof.

Preferably, the BET inhibitor is selected from the group consisting of: I-BET762 (or molibresib), CPI-0610, OTX015, RVX-280 (or apabetalone), ODM-207, PLX-2853, ZEN-3694, ABBV-744, AZD-5153, BI-894999, JQ-1 BOS-475, CC-90010, CC-95775, Mivebresib, BPI-23314, SYHA-1801, ARV-771, CK-103, dBET-1, GSK-3358699, MA-2014, MS-417, NEO-2734, NHWD-870, NUE-7770, OHM-581, PLX-51107, QCA-570, RVX-297, SF-2523, SF-2535, SRX-3177, SRX-3262, ZBC-260, DCBD-005, KM-601, MZ-1, SBX-1301, SRX-3225, ZL-0580, NUE-19796, NUE-20798, or a pharmaceutically acceptable salt, hydrate or solvate thereof and/or combinations thereof.

In the context of the present invention the term “molecule” may comprise also biological agents and oligonucleotide sequences.

As used herein, an “epigenetic regulator” may be any molecule that target an epigenetic regulator or may itself be an epigenetic regulator. When said molecule targets an epigenetic regulator, said epigenetic regulator is preferably defined as any protein able to directly regulate genic transcription through interaction with DNA, RNA or chromatin.

Lysine specific demethylase-1 (LSD1) represents an important class of histone demethylases and has fundamental roles in the development of various pathological conditions. Targeting LSD1 has been recognized as a promising therapeutic option for cancer treatment.

As used herein, DDP38003 corresponds to compound N-[4-[(1S,2R)-2-aminocyclopropyl]phenyl]-4-(4-methylpiperazin-1-yl)benzamide); DDP37368 corresponds to compound N-[4-[(1R,2S)-2-aminocyclopropyl]phenyl]4-(4-methylpiperazin-1-yl)benzamide. “Virus” or “viral” as used herein includes any virus able to perform dsRNA or any RNA virus. Then, it is preferred that the virus is not a DNA virus, such as Herpes virus.

By “coronavirus infection” it is intended an infection caused by or otherwise associated with growth of coronavirus in a subject, in the family Coronaviridae (subfamily Coronavirinae). The virus might affect human and/or other species.

Preferably, the coronavirus is selected from the group consisting of:
(a) alphacoronavirus;
(b) betacoronavirus;
(c) gammacoronavirus; and
(d) deltacoronavirus.

Examples of alphacoronaviruses include Alphacoronavirus 1, Bat coronavirus CDPHE15, Bat coronavirus HKU10, Human coronavirus 229E, Human coronavirus NL63, Miniopterus bat coronavirus 1, Miniopterus bat coronavirus HKU8, Mink coronavirus 1, Porcine epidemic diarrhoea virus, Rhinolophus bat coronavirus HKU2, and Scotophilus bat coronavirus 512. Examples of betacoronaviruses include Murine coronavirus, Betacoronavirus 1, Hedgehog coronavirus 1, Human coronavirus HKU1, Middle East respiratory syndrome-related coronavirus, Pipistrellus bat coronavirus HKU5, Rousettus bat coronavirus HKU9, Severe acute respiratory syndrome-related coronavirus, and Tylonycteris bat coronavirus HKU4.

Examples of gammacoronaviruses include Avian coronavirus and Beluga whale corona virus SW1.

Examples of deltacoronaviruses include Bulbul coronavirus HKU11, Common moorhen coronavirus HKU21, Coronavirus HKU15, munia coronavirus HKU13, Night heron coronavirus HKU19, Thrush coronavirus HKU12, White-eye coronavirus HKU16, and Wigeon coronavirus HKU20.

Preferably, the coronavirus is a human coronavirus, more preferably it is selected from the group consisting of:
(a) human coronavirus 229E;
(b) human coronavirus OC43;
(c) Severe Acute Respiratory Syndrome coronavirus (SARS-CoV)
(d) human Coronavirus NL63 (HCoV-NL63, New Haven coronavirus);
(e) human coronavirus HKU1;
(f) Middle East respiratory syndrome coronavirus (MERS-CoV);
(g) Severe Acute Respiratory Syndrome coronavirus 2 (SARS-CoV-2).

Preferably, the genome of the coronavirus is as defined in: https://www.ncbi.nlm.nih.gov/genomes/GenomesGroup.cgi?taxid=11118, and any database entry linked thereto, e.g. as defined in GenBank with the accession no. NC_034440.1.

Preferably, the nucleotide sequence of the coronavirus and/or the amino acid sequence of the coronavirus proteins are as defined in: https://www.ncbi.nlm.nih.gov/genbank/sars-cov-2-seqs/, e.g. as defined in GenBank with the accession no. MN908947.3.

Preferably, the molecule as defined hereinabove is not one of those disclosed in D. E. Gordon et al., “A SARS-CoV-2-Human Protein-Protein Interaction Map Reveals Drug Targets and Potential Drug-Repurposing” doi: https://doi org/10.1101/2020.03.22.002386, herein incorporated by reference.

The term “treatment” comprises the alleviation, in part or in whole, of the symptoms of the viral infection, preferably the Coronavirus infection (depending on the particular type of coronavirus, and on the stage of the infection, the symptom may include but not being limited to elevated body temperature, sore throat, blocked and/or runny nose, cough, anosmia and other sensory deficits, respiratory distress associated with pneumonia which may require artificial ventilation and require intensive care therapy). Such treatment may include eradication, or slowing of the Coronavirus growth, and may include the eradication or slowing the growth of other viral agents or of other microbial agents which co-associated with the Coronavirus infection. Such treatment may lead to disappearance or amelioration of the symptoms associated with Coronavirus infection, including but not being limited to the effect of treatment being that of blocking the worsening of the subject symptoms which required hospitalization, artificial ventilation, and recovery in intensive care units.

The term “prevention” includes the reduction in risk of the viral infection and/or of the viral disease, preferably of coronavirus in patients. However, it will be appreciated that such prevention may not be absolute, i.e. it may not prevent all such patients developing a coronavirus infection, slowing down the infection and/or attenuating its symptoms as above. As such, the terms “prevention” and “prophylaxis” may be used interchangeably.

In one embodiment, the Coronavirus infection is an infection of the upper and/or lower respiratory tract. Alternatively, or additionally, the Coronavirus infection may be in the gastrointestinal tract, or affect other organs (such as the central nervous system). Certain coronavirus, such as MERS, can also infect renal epithelial cells. In all cases, Coronaviruses may infect target cells through binding to specific receptors. In the case of certain Coronaviruses (SARS-CoV, MERS, SARS-CoV-2) the receptor is the ACE2 receptor. As such, the coronavirus infection is an infection of any organ which contains cells expressing the ACE2 receptor in its parenchymal cells, or in cells of the connective tissue (vascular cells, fibroblast cells).

The term “upper respiratory tract” includes the mouth, nose, sinus, middle ear, throat, larynx, and trachea. The term “lower respiratory tract” includes the bronchial tubes (bronchi) and the lungs (bronchi, bronchioles and alveoli), as well as the interstitial tissue of the lungs.

By “gastrointestinal tract” it is intended the canal from the mouth to the anus, including the mouth, oesophagus, stomach and intestines.

In an alternative embodiment the Coronavirus infection is a renal infection.

The present invention also comprises analogs, tautomeric forms, stereoisomers, polymorphs, solvates, intermediates, pharmaceutically acceptable salts, metabolites, and prodrugs of the molecules herein described.

Salts of the herein described molecules are within the scope of the present invention. As used herein, the term “salt” refers to an acidic and/or basic salt formed with inorganic and/or organic acids and bases. Salts of the compounds of the present invention may be formed, for example, by reacting a compound of the present invention with an equivalent amount of an acid or base in an aqueous medium or in a medium such as one in which a salt precipitates.

Non-limiting examples of such salts include the following: for example, acetic acid, adipic acid, benzenesulfonic acid, benzoic acid, camphoric acid, camphorsulfonic acid, citric acid, cyclamic acid, ethane-1,2-disulfonic acid, ethanesulfonic acid, 2-hydroxyethanesulfonic acid, formic acid, fumaric acid, bromic acid, hydrochloric acid, sulfuric acid, nitric acid, phosphoric acid, malic acid, maleic acid, methanesulfonic acid, naphthalene-2-sulfonic acid, naphthalene-1,5-disulfonic acid, 1-hydroxy-2-naphthalene acid, nicotinic acid, trifluoroacetic acid, oxalic acid, p-toluenesulfonic acid, propionic acid, glycolic acid, succinic acid, tartaric acid, amino acid (e.g., lysine), salicylic acid, 2,2-chloroacetic acid, L-aspartic acid, (+)-(1S)-camphor-10-sulfonic acid, 4-acetamidobenzoic acid, caproic acid, cinnamic acid, gentisic acid, glutaric acid, malonic acid, mandelic acid, orthic acid, pamoic acid, aminosalicylic acid and the like may be used to form an acid addition salt. When a plurality of basic groups are present, they may form mono- or poly-acid addition salts.

In addition, the molecules of the invention may be employed in unsolvated as well as in solvated forms with pharmaceutically acceptable solvents such as water, EtOH and the like. Preferably, said solvate is a hydrate.

Molecules herein disclosed may contain one or more asymmetric carbon atoms. The individual stereoisomers (enantiomers and diastereomers) as well as mixtures of these are included within the scope of the present invention.

Likewise, it is understood that molecules of the invention may exist in tautomeric forms and all of these are also included within the scope of the present invention.

Included in the present invention are “pharmaceutically acceptable derivative”, i.e. pharmaceutically acceptable salts, hydrates, solvates, prodrugs, complexes, stereoisomers or enantiomers of the molecules of the present invention, that maintain the desired biological activity of the molecules and minimally exhibit or do not exhibit undesirable toxicological effects In addition, the present invention includes prodrugs of the compounds of the present invention. The term “prodrug” is intended to indicate a compound that is covalently bonded to a carrier (vehicle), and when the prodrug is administered to a mammalian subject, an active ingredient may be released. Release of the active ingredient may occur in vivo. Prodrugs may be prepared by techniques that would be known to those skilled in the art. An appropriate functional group in a certain compound is modified by these techniques. Such a modified functional group regenerates the original functional group in routine manipulation or in vivo. Examples of prodrugs include, but are not limited to, esters (e.g., acetate, formate and benzoate derivatives) and the like.

A typical suitable dosage of the molecules of the present invention required for treatment as a single dose or separation dosage is in the range of about 0.001 to 750 mg per kg of body weight per day, in particular 0.001 to 100 mg per kg of body weight per day, preferably 0.001 to 10 mg, most preferably in the range of 0.005 to 5 mg per kg of body weight. However, a specific dose level for an individual patient may vary depending on the particular compound to be used, the body weight, sex and diet of the patient, time of administration of a drug, method of administration, rate of excretion, drug mix, condition and age of the patient, and the like.

The pharmaceutical composition as disclosed above may further comprise a pharmaceutically acceptable carrier. For example, said carrier may be inert and may be selected from, but are not limited to, fillers such as sugar including lactose, dextrose, sucrose, sorbitol, mannitol, xylitol, erythritol and maltitol; starch including corn starch, wheat starch, rice starch and potato starch; cellulose family including cellulose, methyl cellulose, sodium carboxymethyl cellulose and hydroxypropyl methylcellulose; gelatin, polyvinylpyrrolidone and the like. In addition, in some cases, a disintegrant such as crosslinked polyvinylpyrrolidone, agar, alginic acid or sodium alginate may be added, but the disintegrant is not limited thereto.

For instance, the pharmaceutical composition may further include, but is not limited to, an anti-cohesive agent, a lubricant, a wetting agent, a flavor, an emulsifier and a preservative.

In addition, the compounds or pharmaceutical compositions of the present invention can be administered by any route as desired. The compounds or pharmaceutical compositions can be administered orally or parenterally, and examples of the parenteral administration route include, but are not limited to, various routes such as transdermal, nasal, peritoneal, muscular, subcutaneous, intravenous injection and the like. Specifically, the administration route of the compounds or pharmaceutical compositions of the present invention is preferably injection and oral administration.

The injectable preparations, for example, a sterilized injectable aqueous or oleaginous (oily) suspension, may be prepared using suitable dispersing agents, wetting agents or suspending agents according to the known technique. Solvents that may be used for this purpose include water, Ringer's solution and isotonic NaCl solution, and sterilized, fixed oils are also conventionally used as a solvent or suspending medium. Any non-irritating fixed oils, including mono- or di-glycerides, can be used for this purpose, and fatty acids such as oleic acid can also be used in injectable preparations.

Solid dosage forms for oral administration include capsules, tablets, pills, powders and granules, and capsules and tablets are especially useful.

Tablets and pills are preferably prepared as enteric-coated ones. Solid dosage forms can be prepared by admixing a molecule according to the present invention with a carrier such as one or more inert diluents including sucrose, lactose, starch, etc.; lubricants such as magnesium stearate; disintegrants, binders and the like.

In one embodiment of the present invention, the above molecules or pharmaceutically acceptable salts thereof, or the pharmaceutical compositions comprising the same, may be administered in combination among themselves, to achieve improved antiviral efficacy and/or reduced toxicity.

In one embodiment of the present invention, the above molecules or pharmaceutically acceptable salts thereof, or the pharmaceutical compositions comprising the same, may be administered in combination with one or more additional agents to achieve therapeutic exposures and biological activity, such as a cytidine-deaminase inhibitor.

In one embodiment of the present invention, the above molecules or pharmaceutically acceptable salts thereof, or the pharmaceutical compositions comprising the same, may be administered in combination with one or more additional agents having antiviral efficacy to prevent and treat respiratory viral infectious diseases.

For example, the pharmaceutical compositions may be administered in combination with one or more antiviral drugs such as, Interferon, Ribavirin or Lopinavir/Ritonavir, chloroquine, hydroxychloroquine.

In one embodiment of the present invention, the above molecules or pharmaceutically acceptable salts thereof, or the pharmaceutical compositions comprising the same, may be administered in combination with one or more additional agents which may (administered in combination) improve efficacy and/or reduce toxicity, for example retinoic acid, lipoic acid, heparin, anti-coagulating agents, Vitamin D, antibiotics, corticosteroids.

In one embodiment, the combination can be administered simultaneously, separately or sequentially (in any order).

The invention will be illustrated by means of non-limiting examples in reference to the following figures.

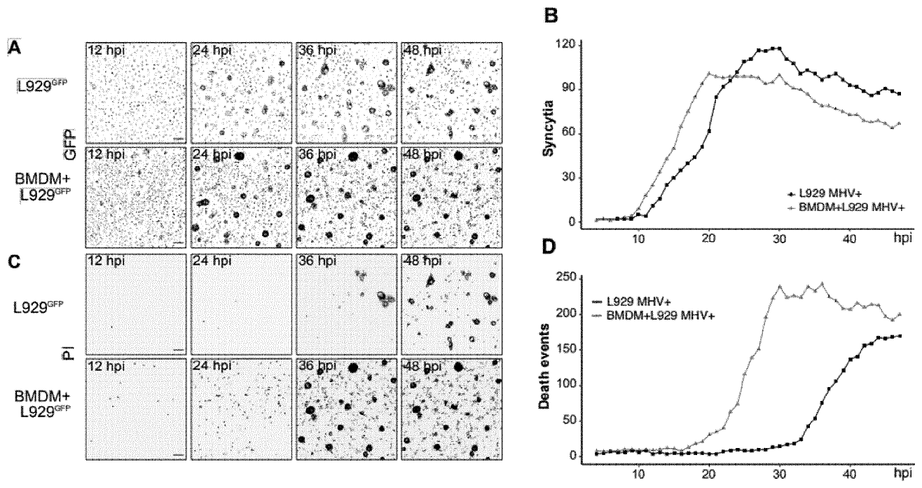

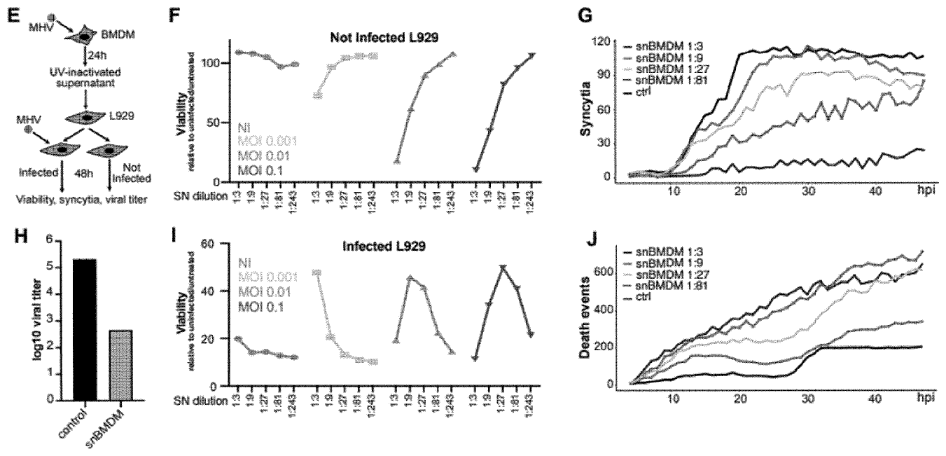

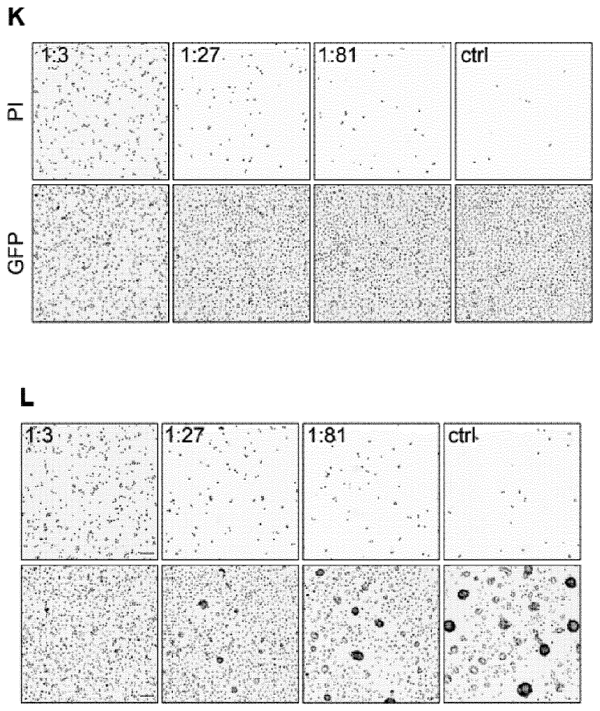

FIG. 1. Description of the experimental system

A, C: Representative snapshots from time lapse microscopy imaging of L929GFP cells alone or cocultured with BMDM at a 1:2 ratio. Cells were infected at MOI 0,1. hpi: hours post infection. A, GFP signal, C: PI signal (cell death). Fluorescence signals were visualized with the inverted grey look up table (LUT). Scale bars: 100 μm.

B, D. Time course of syncytia formation (B) and death events occurrence (D), monitored by time lapse microscopy, of L929GFP cells alone or cocultured with BMDM at a ratio 1:2 respectively. Cells were infected at MOI 0.1. hpi: hours post infection.

E. Experimental strategy to evaluate BMDM extrinsic activity.

F,I: Cell viability of uninfected (F) and infected (I) L929 cells exposed to BMDM supernatants at three-fold dilutions (from 1:3 to 1:243). BMDM supernatants were collected from not infected (NI) or MI-IV infected BMDM at different MOI (MOI 0,1-0,001). Viability was measured by Cell Titer Glo in triplicate and expressed as Relative Luminescence Units (RLU) compared to uninfected and untreated cells.

G,J: Time course of syncytia formation (G) and death events occurrence (J), monitored by time lapse microscopy, of MHV-infected L929GFP cells (MOI 0.1) exposed to the supernatant of MHV infected BMDM (MOI 0.1) at the indicated dilutions. Normal medium was used as control (CTRL).

H: viral titer from L929 cells treated (sn BMDM) or not (control) with supernatant from MHV-infected BMDM (MOI 0.1) at 1:3 dilution. Viral titers were measured by TCID50, 24 hours post infection.

K,L: Representative snapshots from time lapse microscopy imaging at 24 hours of L929GFP cells not infected (K) or MHV infected at 0.1 MOI (L), exposed to MHV infected BMDM supernatants at the indicated dilutions. Scale bars: 100 μm.

Abbreviations: NI=not infected, I=infected, PBS=Phosphate Buffered Saline (added in place of supernatant as negative control in fractionation experiments), NE=Not Eluted, RLU=Relative Luminescence Units

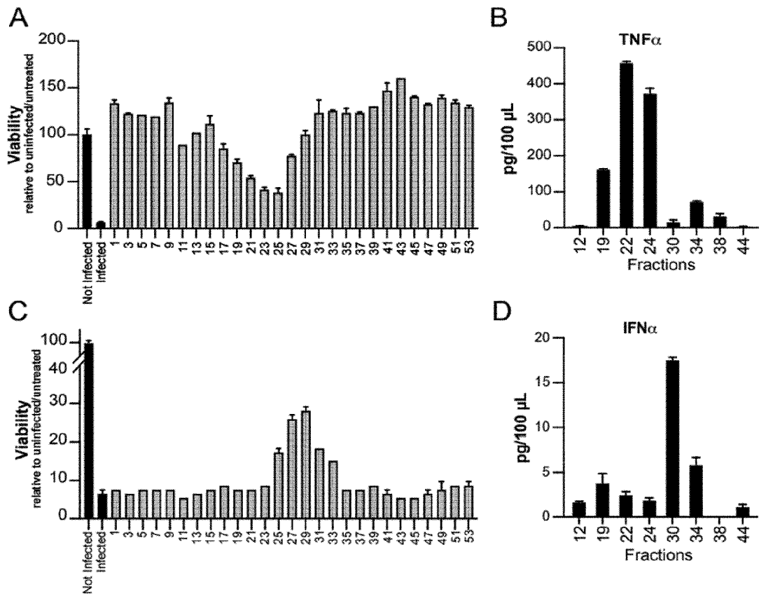

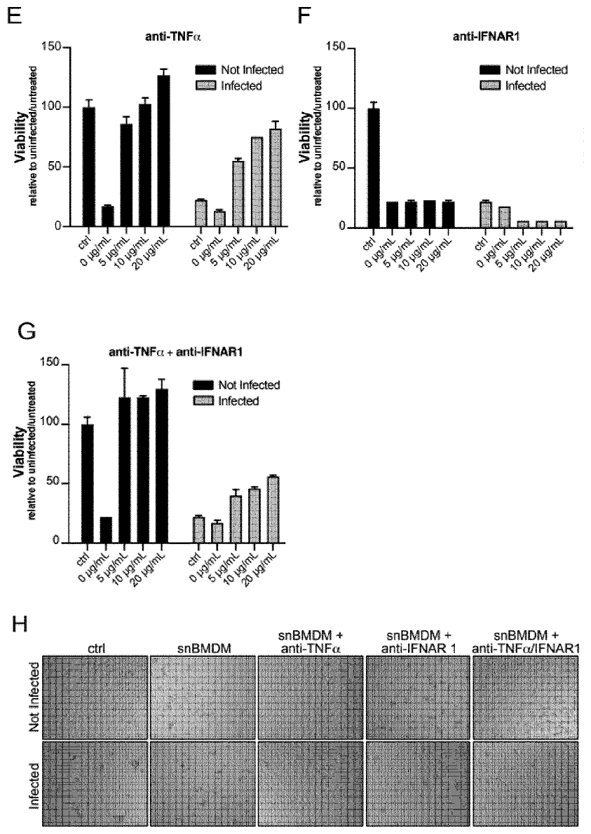

FIG. 2. BMDM-secreted cytotoxic and antiviral activities are biochemically distinct and are sustained by TNFa and IFNa

A,C: Effect of size-fractionated supernatant from MI-IV-infected (MOI 0.1) BMDMs on viability of non-infected (A) or infected MOI 0.1 (C) L929 cells. Viability is measured by Cell Titer Glo in triplicate (shown is mean+SD) and is expressed as fraction of uninfected and untreated cells.

B, D. ELISA for TNFα and IFNα on size-fractionated supernatant from MHV-infected (MOI 0.1) BMDMs. Mean±SD of triplicate wells per experiment.

E-G. Effect of increasing doses of TNFα- or IFNα-neutralizing antibodies, alone or in combination, on 48h viability of non-infected or infected MOI 0.1 L929 cells exposed to supernatant from MOI 0.1-infected BMDM. Mean±SD of triplicate wells per experiment.

H. representative bright field images of non-infected or infected MOI 0.1 L929 cells 24 hours after treatment with control, supernatant from infected BMDM with or without TNFα- or IFNα-neutralizing antibodies, alone or in combination.

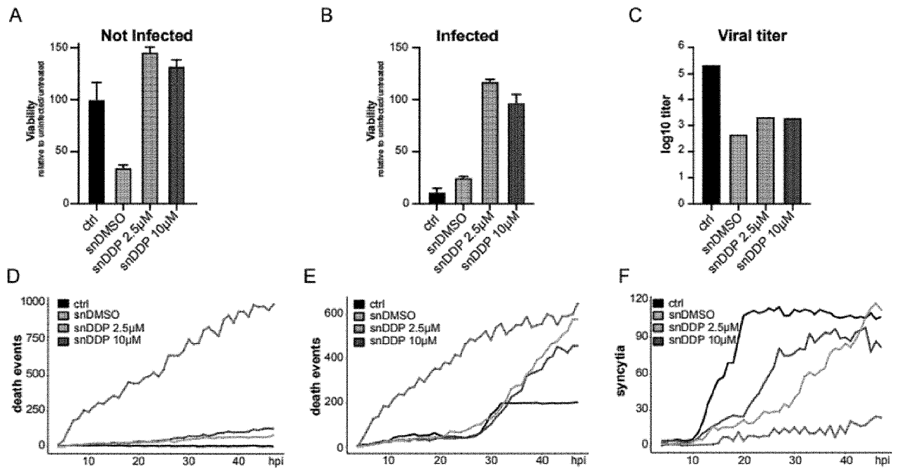

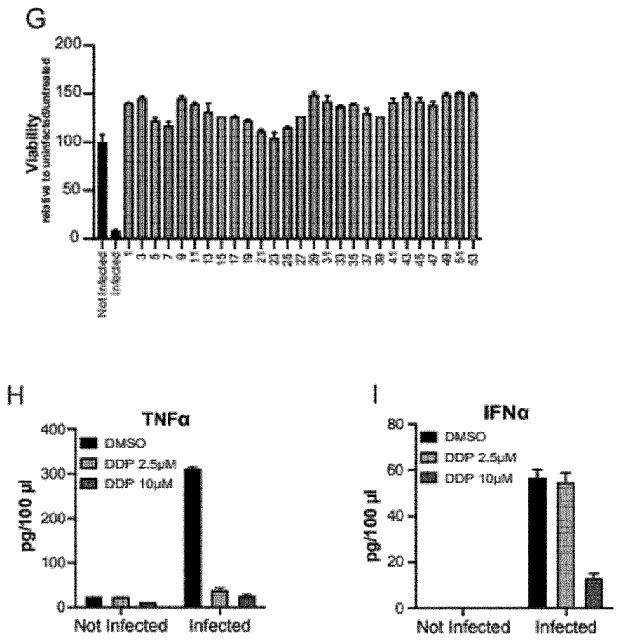

FIG. 3. LSD1 inhibition ablates extrinsic cytotoxic activity but preserves extrinsic antiviral activity

A-F. Effect of LSD1 inhibitor DDP38003 on BMDM supernatant. BMDM were treated with vehicle (snDMSO) or LSD1 inhibitor DDP38003 (snDDP) at medium (2.5 μM) or high (10 μM) doses, 24 hours prior to infection with MHV-A59 at MOI 0.1. Supernatant was collected from BMDM cultures 24 hpi and applied to infected or uninfected L929 or L929^{GFP} cells.

A: 48h viability of uninfected L929 cells measured by CTG; mean±SD of triplicate wells.

B: 48h viability of infected L929 cells measured by CTG (for A and B mean and standard deviation of triplicate wells of one representative experiment are shown); mean±SD of triplicate wells.

C. Viral titer measured by TCID50 24 hpi on L929 cells.

D number of death events in time lapse imaging of uninfected L929^{GFP} cells

E. number of death events in time lapse imaging of infected L929^{GFP} cells

F. number of syncitia in time lapse imaging of infected L929^{GFP} cells live cell imaging

G-J. size-exclusion chromatography on supernatant from BMDM infected with MOI 0.1 and treated with DDP 2.5 μM. Biological activity of individual fractions was measured as in FIG. 2 on uninfected (G) or MOI 0.1-infected (J) L929 cells.

H-I. ELISA for TNFα (H) and IFNα (I) on supernatant from BMDM infected or not with MHV 0.1 MOI and treated with vehicle (DMSO) or DDP at medium or high dosage. Mean±SD of triplicate wells per experiment.

K,L. ELISA for TNFα and IFNα on size-fractionated supernatant from BMDM infected with MHV 0.1 MOI and treated with vehicle (DMSO) or DDP at medium or high dosage. Mean±SD of triplicate wells per experiment.

M. representative bright field images of non-infected or infected MOI 0.1 L929 cells 24 hours after treatment with control or supernatant from MOI 0.1-infected BMDM, treated with DDP 2.5 μM and treated with or without TNFα- or IFNα-neutralizing antibodies, alone or in combination.

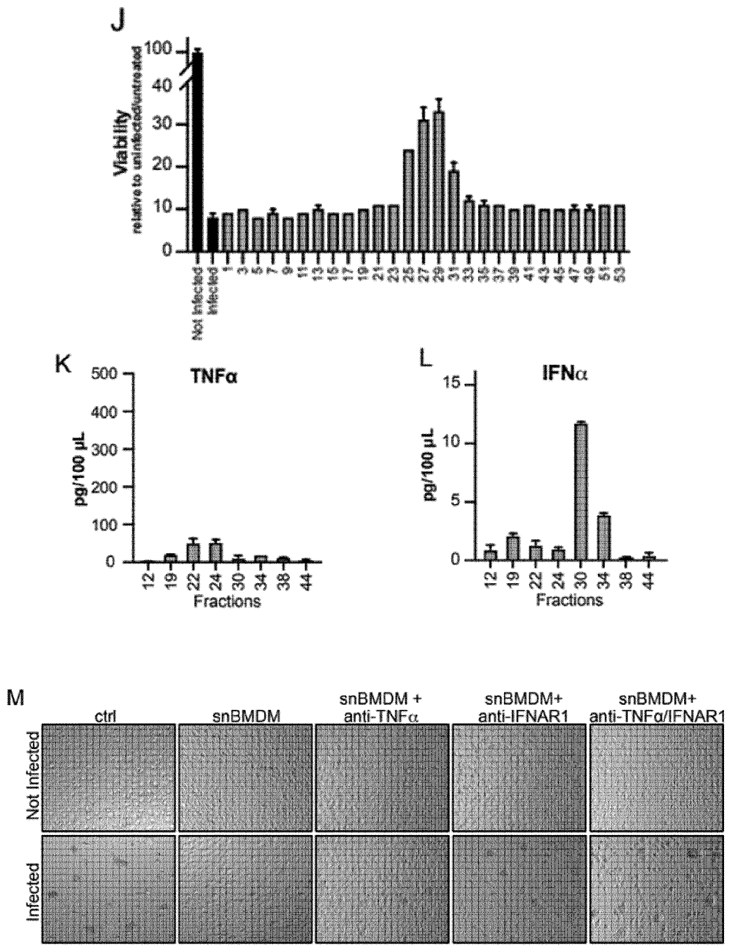

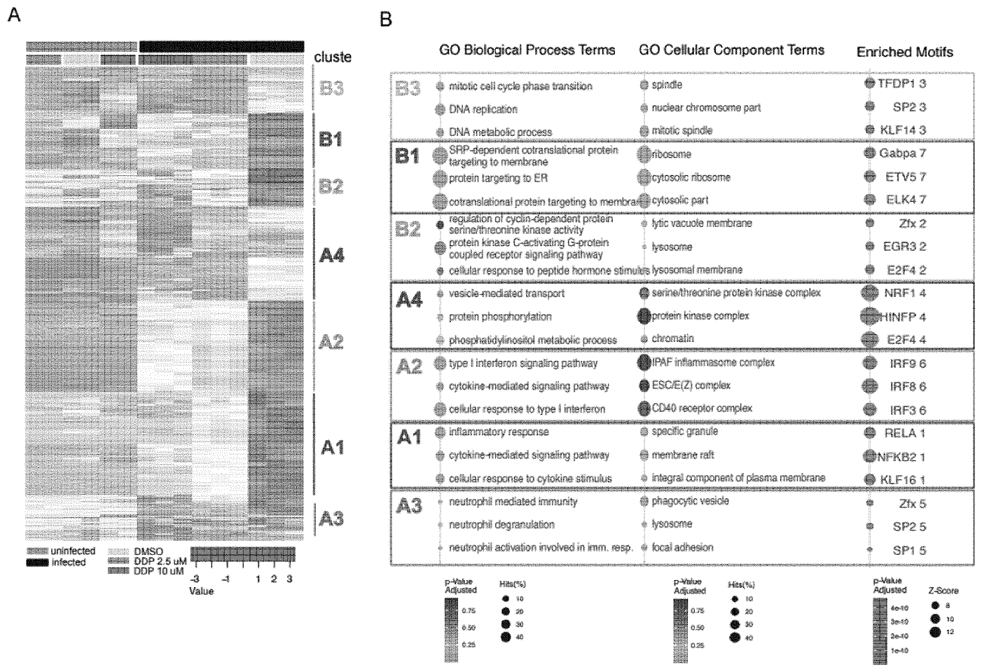

FIG. 4. Effect of MHV infection and DDP treatment on macrophage transcriptional response

A. Hierarchical clustering of RNAseq data of BMDM infected or not with MHV-A59 MOI 0.1 and treated with DMS, DDP 2.5 μM or DDP 10 μM. Noninfected cells are in duplicate, infected in triplicate

B. Gene ontology and transcription factor enrichment by cluster

C. boxplot of log 2-fold changes of genes within each cluster over uninfected, untreated cells. Color code as in FIGS. 4A,B.

D, E. Gene expression analysis by qRT-PCR of representative NF-KB target genes (D) and IFNα and representative ISG (E) over the first 24 hpi in BMDM infected or not with MHV-A59 MOI 0.1 and treated with DMSO, DDP 2.5 μM or DDP 10 μM.

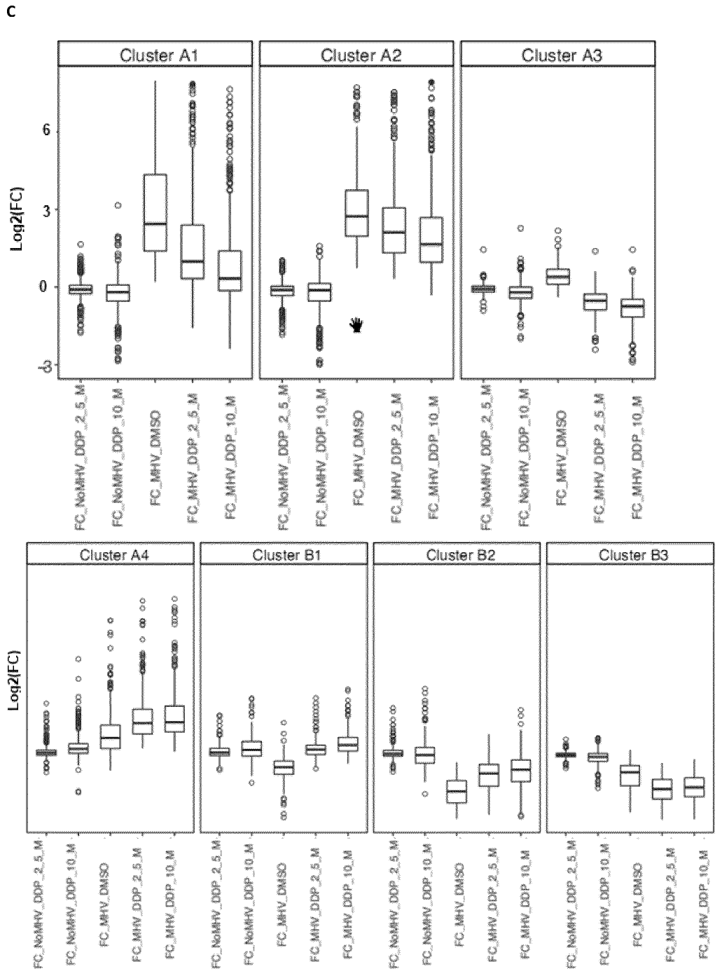

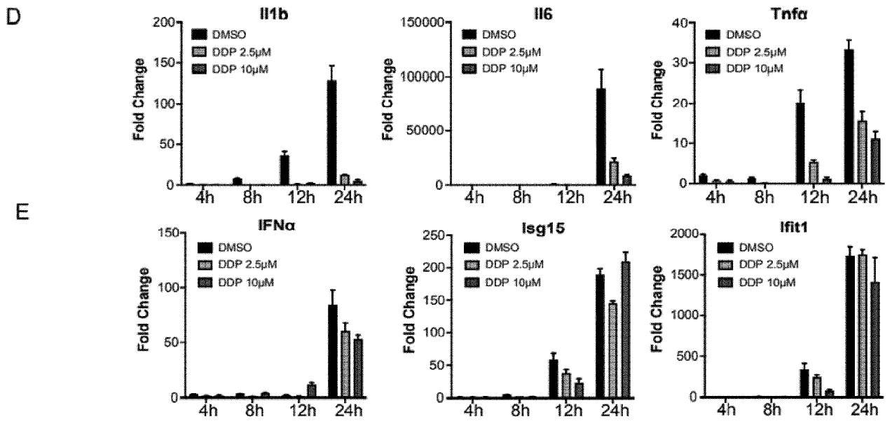

FIG. 5. LSD1 inhibition abrogates NFKB nuclear translocation more than IRF1 nuclear translocation in response to MHV-A59 infection

A-B WB of whole cell lysate (WCL), cytoplasmic (C) and nuclear (N) fractions of BMDM at 10 hpi MOI 5 (A) and 24 hpi MOI 0.1 (B)

C, D. IF for NFKB and IRF1 (D) showing subcellular localization in response to MHV; NSP9 identifies infected cells. In C representative images at 10 hpi MOI 5 are shown. In D, boxplot of the mean nuclear signal for NFKB (upper panel) or IRF1 (lower panel), divided by NSP9 positivity and treatment. Dashed line indicates the signal of uninfected and untreated cells at baseline

E. Chromatin IP for NFKB and LSD1 in BMDM infected with MHV-A59 MOI 5 at 10 hpi, treated with DMSO or DDP 2.5 μM. Mean±standard deviation of triplicate PCRs

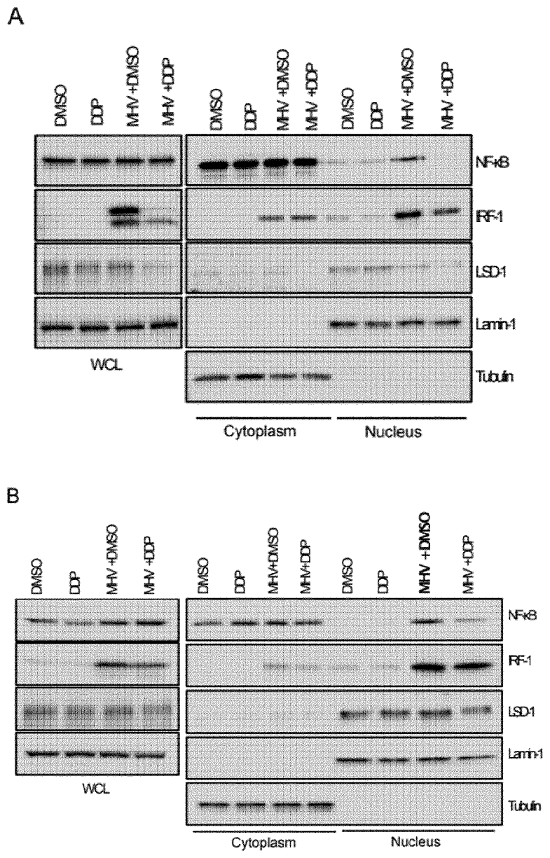

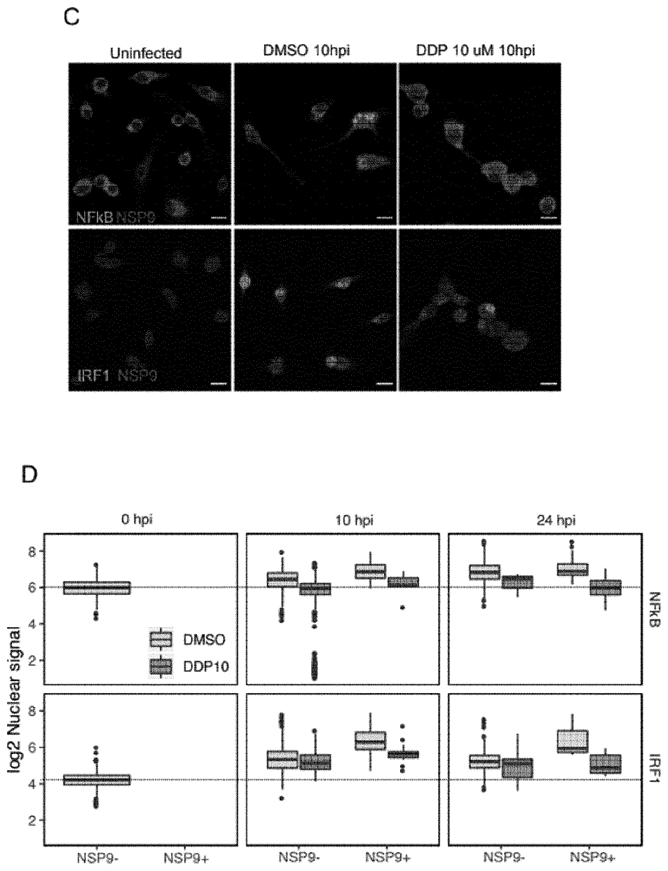

FIG. 6. LSD1 inhibition exerts intrinsic antiviral activity and enhances lysosomal acidification

A-D. Direct antiviral effect on L929 cells, measured as survival by CTG (A), death events (B) and live cells (C) by live cell imaging, viral titer at 24 hpi (D) and syncitia formation by live cell imaging on L929^{GFP} cells.

E-F. Effect of LSD1 KO (2 independent clones KO1 and KO2 vs non-targeted clone WT) on 48 hpi viability (E) or 24 hpi titer (F) of L929 cells infected with MHV at indicated MOI.

G. Expression of selected ISGs by qRT-PCR in response to MHV-A59 MOI 0.1 and DMSO or DDP at increasing doses

H. Lysosome pH measurement using lysosensor on L929 cells uninfected or infected with MHV-A59 MOI 5 and treated with DMSO or DDP 10 μM, at 12 hpi. Representative images (left) and quantification (right). All distributions are significantly different by pairwise comparison with p values <0.0001 by Mann-Whitney test (distributions are non-normal) and adjusted by Benjamini-Hochberg

I. BiP extrusion in L929 cells uninfected or infected with MHV-A59 MOI 5 and treated with DMSO or DDP 10 μM at 12 hpi, measured by WB on cell pellets and 100 ul supernatant). The GST peptide was added as spike to the supernatant to equalize supernatant loading

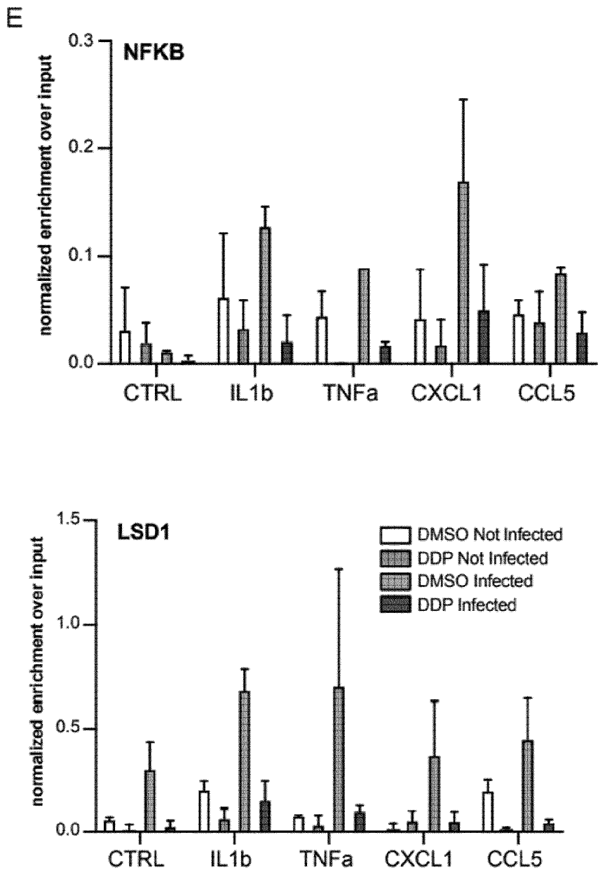

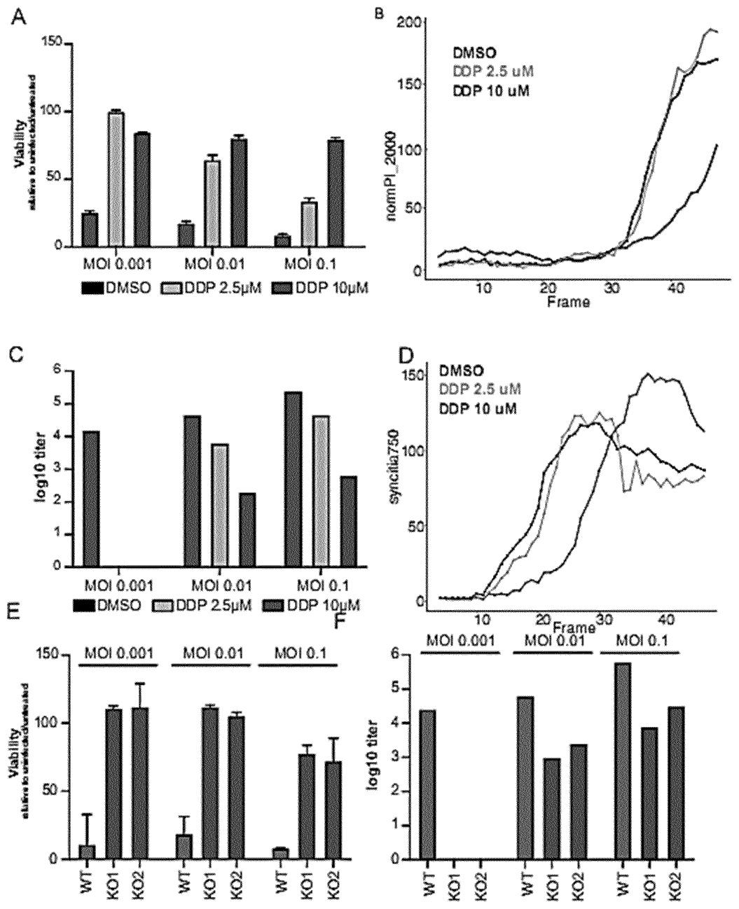

FIG. 7. LSD1 inhibition is a viable target for human Covid19 treatment

A-D response to equimolar doses of DDP38003 (DDP), Ory1001(ORY) and dexamethasone (DEXA) on macrophage extrinsic activity (A), cell survival of L929 cells and viral titer of L929 cells (C); representative bright field images of L929 cells infected at MOI 0.1 and treated as indicated are shown in D

E. Expression level by qRT-PCR of NF-KB and ISGs in human macrophages stimulated with TLR3/RIGI/MDA5 agonist polyIC and TLR7/8 agonist R848.

F. Model depicting innate responses to coronaviruses regulated by LSD1

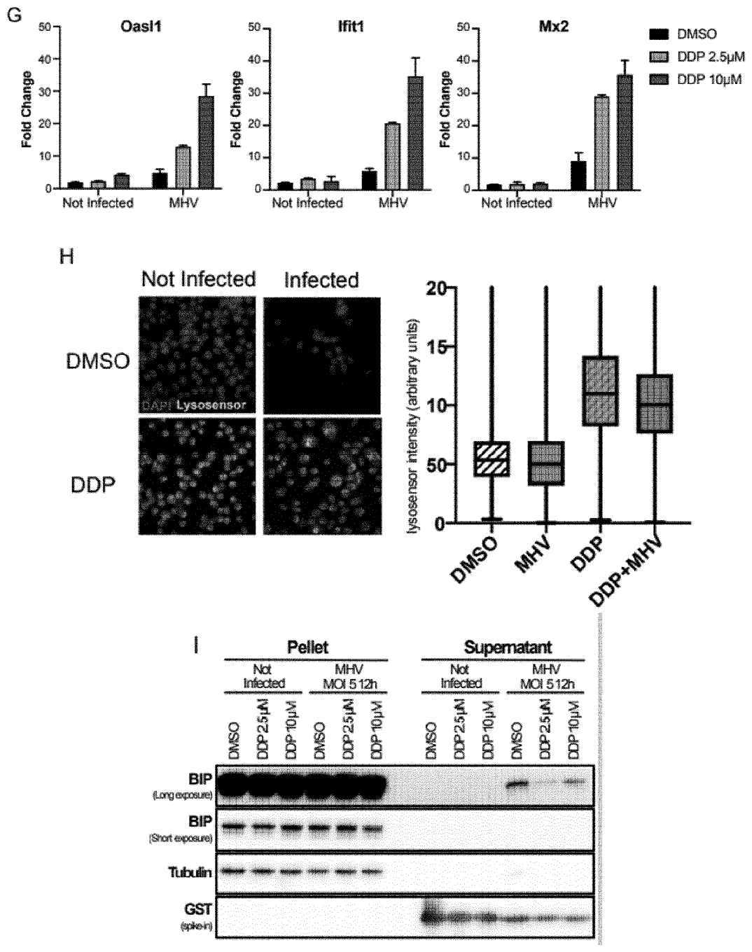

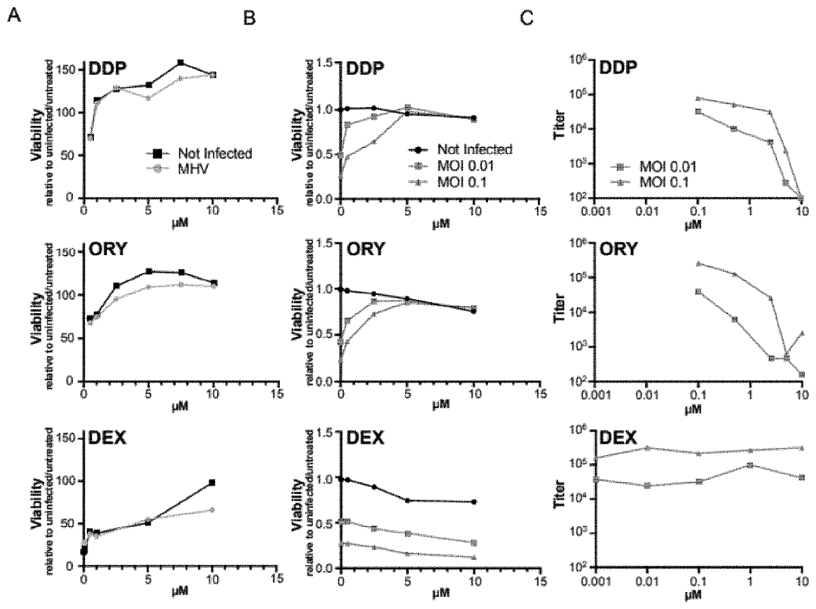

FIG. 8

A. Viral titer 24 hpi in BMDM, L929 and 2:1 cocultures, by MOI, measured by TCID50

B. Cell viability 48 hpi measured by CTG in BMDM, L929 and 2:1 cocultures, by MOI. Mean and standard deviation of triplicate wells, expressed as percentage relative to uninfected cells

C. representative bright field images of mono- and cocultures 48 hpi. Scale bars: 100 μM.

D. TCID50 of L929^{GFP} vs L929^{WT} cells

E. Death events overlapping with syncitia or non-syncitia in L929^{GFP} monocultures (upper panel) or 2:1 BMDM:L929 cocultures (bottom panel)

F. Time course of death events in time lapse microscopy, of uninfected L929^{GFP} cells (MOI 0.1) exposed to the supernatant of MI-IV infected BMDM (MOI 0.1) at the indicated dilutions. Normal medium was used as control (CTRL)

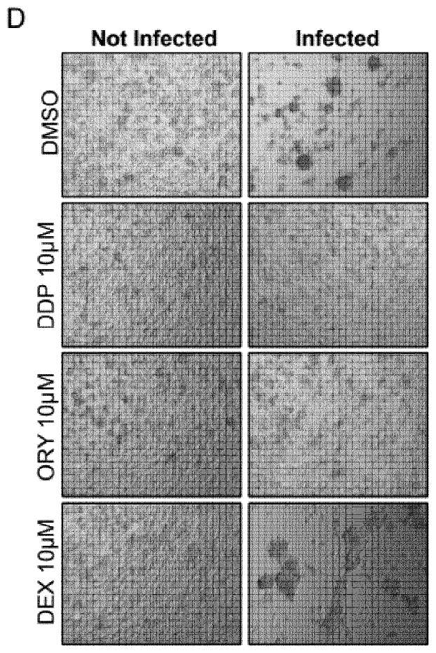

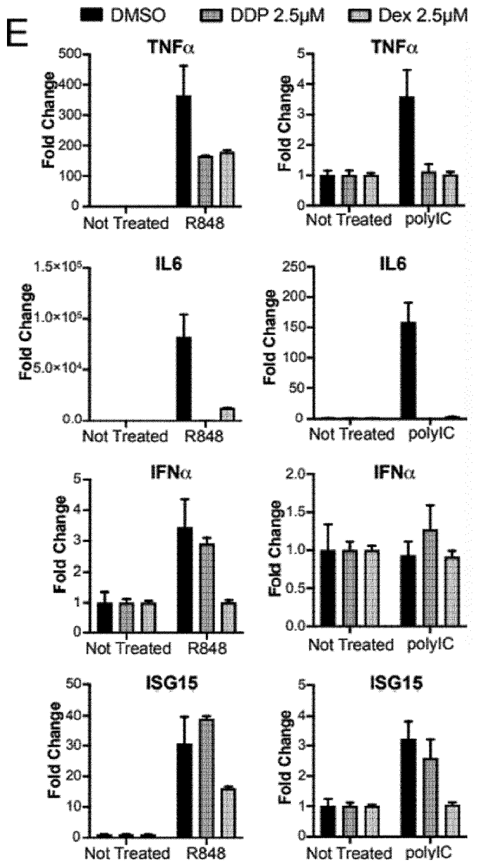

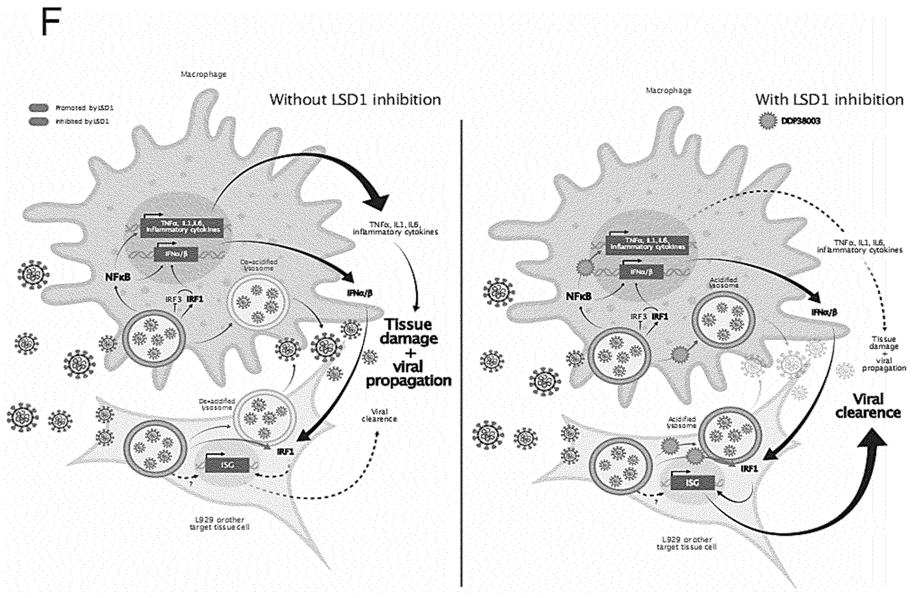

FIG. 9.

Effect of increasing doses of JAK inhibitor I, on 48h viability of non-infected or infected MOI 0.1 L929 cells. Mean±SD of triplicate wells per experiment

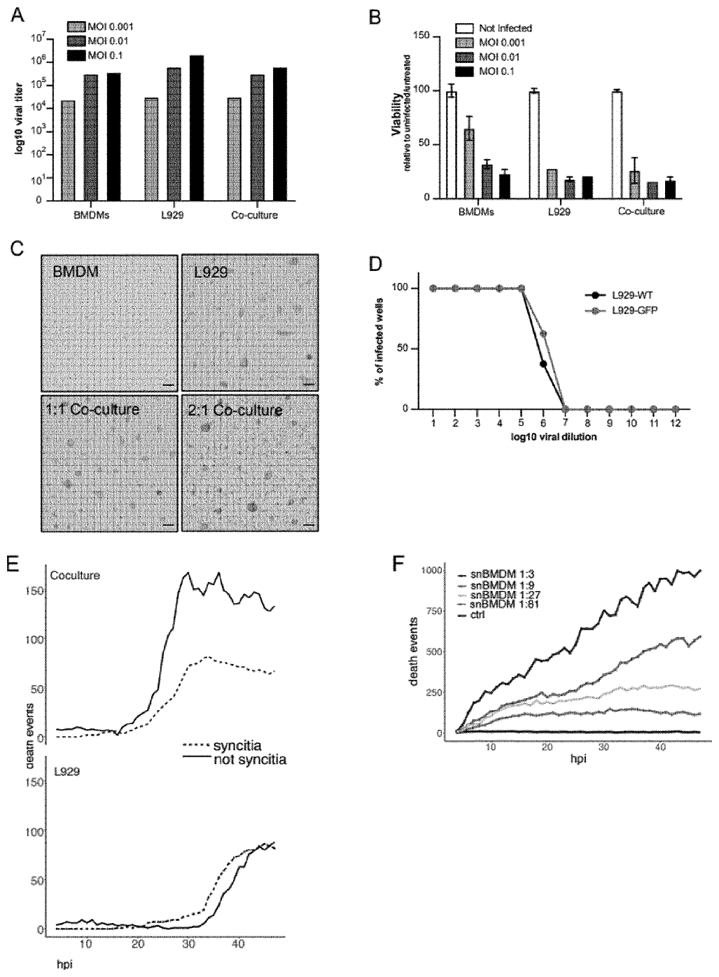

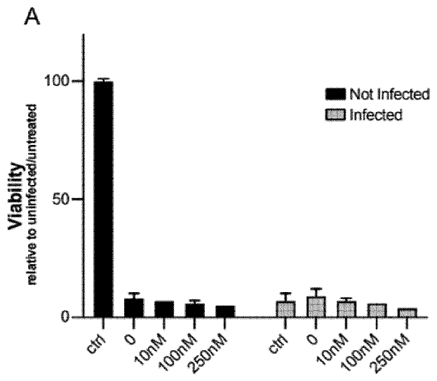

FIG. 10

A. Effect of DDP38003 on viability of L929 and BMDM cells

B-E. Effect of increasing doses of anti-TNFα (B), anti-IFNα (C), anti-TNFα+anti-IFNα (D) or JAK inhibitor I (E), on 48h viability of non-infected or infected MOI 0.1 L929 cells exposed to supernatant from MOI 0.1-infected BMDM treated with DDP 2.5 or 10 μM. Mean±SD of triplicate wells per experiment

F. Efficiency of shLSD1 on LSD1 protein levels in RAW264.7 cells

G. Viral titer 24 hpi with MI-IV-A59 MOI 0.1 in Raw 264.7 cells, measured by TCID50

H. Viability of infected (MOI 0.1) or uninfected L929 cells exposed for 48 hours to empty medium (control) or snBMDM scramble and shLSD1 infected RAW264.7 cells

I. Viability of infected (MOI 0.1) or uninfected LA4 cells exposed for 48 hours to empty medium (control) or supernatant from BMDM MOI 0.1 infected and treated with DMSO or DDP 2.5 μM

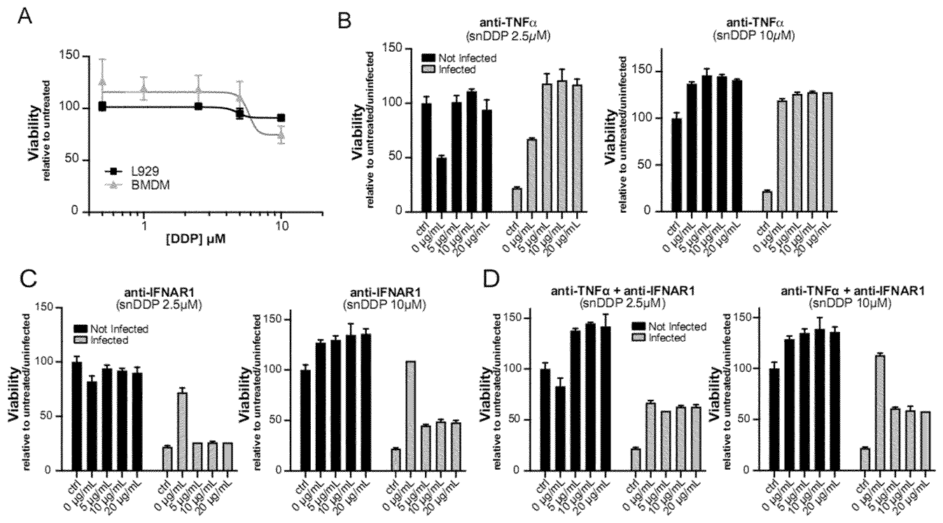

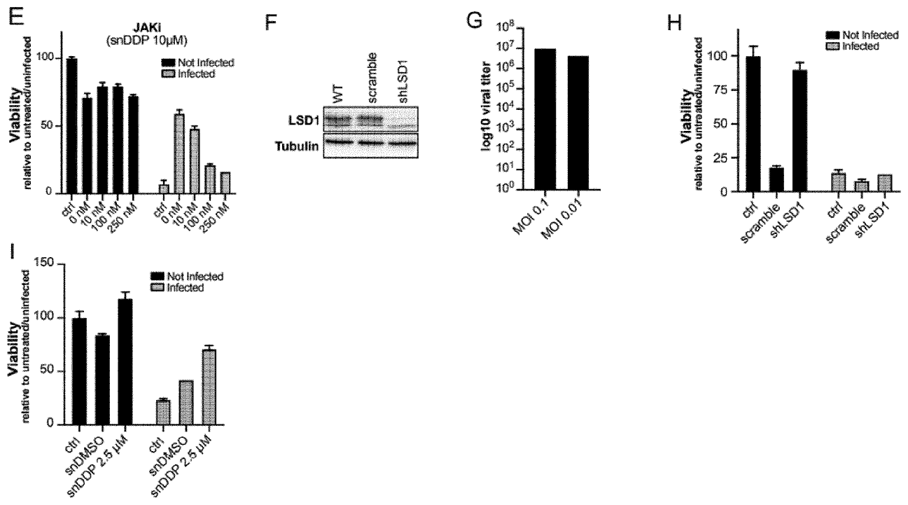

FIG. 11

A. Volcano plots of indicated contrasts in BMDM infected or not with MI-IV-A59 MOI 0.1 at 24 hpi and treated with vehicle (DMSO) or DDP at 2.5 or 10 μM

B. Upset plot showing overlap between genes in BMDM clusters and clusters in Kim et al.

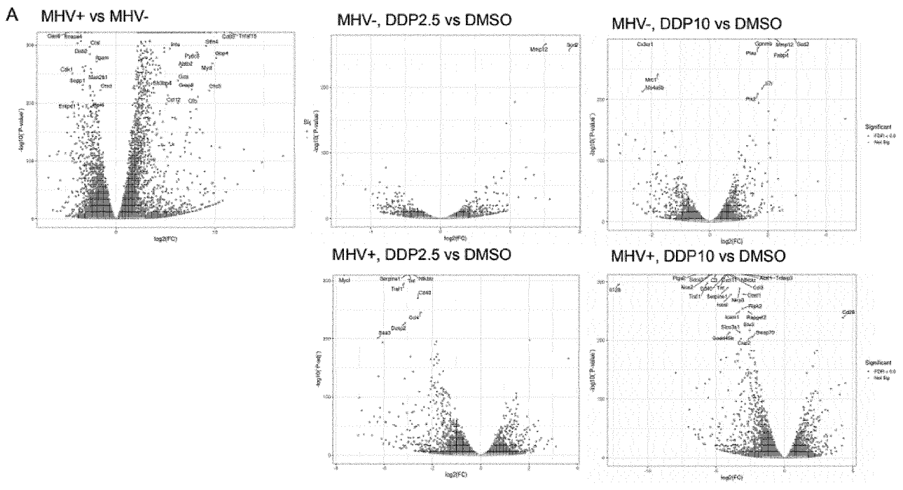

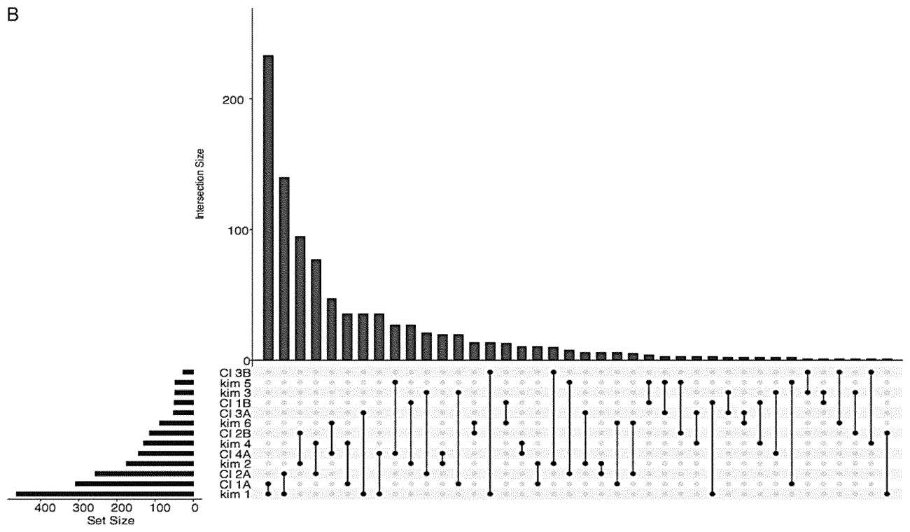

FIG. 12

A. Expression of IRF factors in nuclear extracts of infected BMDM at 8h (MOI 5) and 24h (MOI 1), by Western Blotting

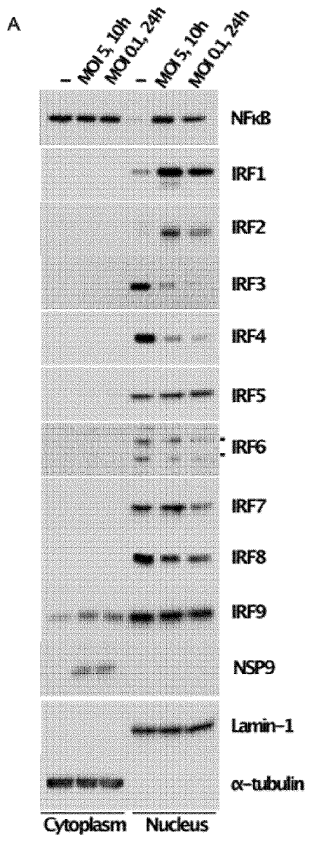

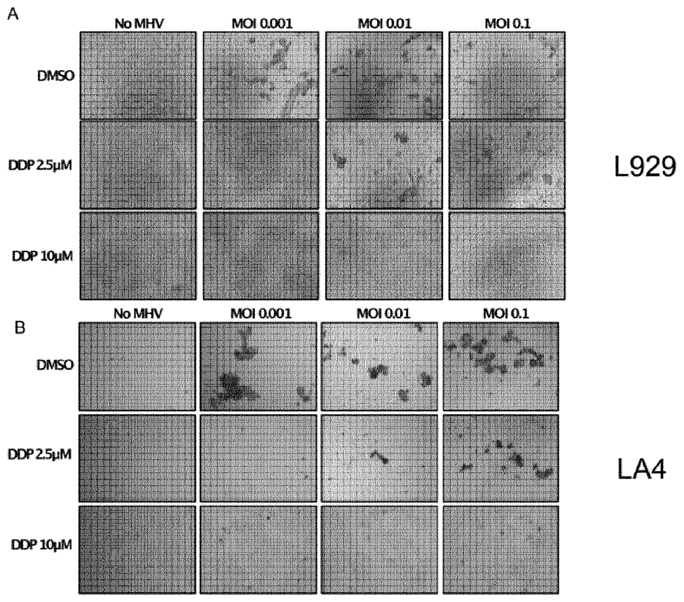

FIG. 13

A. Bright field image of L929 cells infected with MHV-A59 and treated with DDP at indicated doses and MOI

B. Bright field image of LA4 cells infected with MHV-A59 and treated with DDP at indicated doses and MOI

C. Survival (measured by Cell Titer Glow) of LA4 cells infected with MHV-A59 and treated with DDP at indicated doses. Mean±SD of triplicate wells per experiment

D. Viral titer (TCID50) of LA4 cells infected with MHV-A59 and treated with DDP at indicated doses

E. Survival (measured by Cell Titer Glow) of BMDM infected with MHV-A59 and treated with DDP at indicated doses. Mean±SD of triplicate wells per experiment

F. Viral titer (TCID50) of BMDM infected with MHV-A59 and treated with DDP at indicated doses and MOI

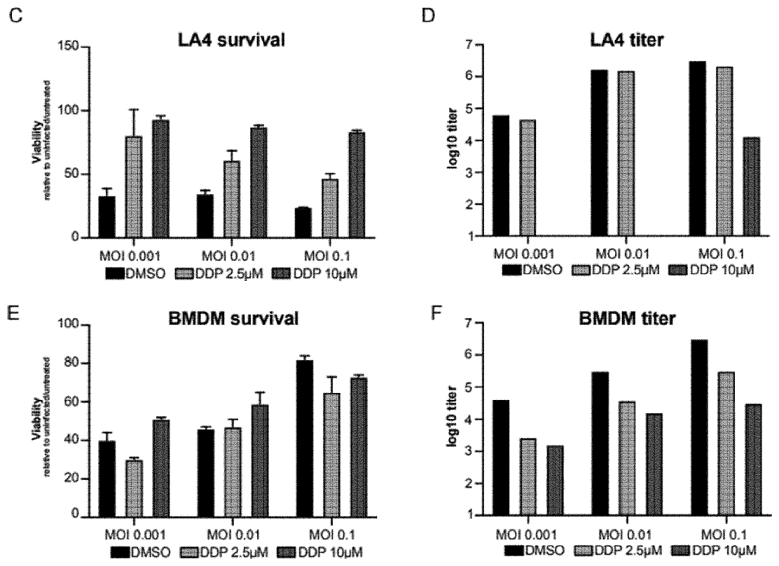

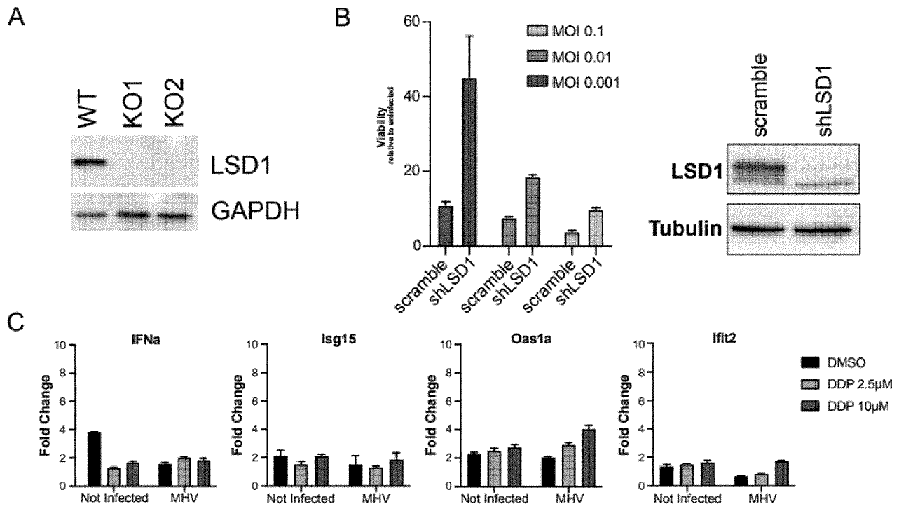

FIG. 14

A. Western Blot showing ablation of LSD1 protein in L929 cells by CRISPR-Cas9

B. Effect of LSD1 knockdown on L929 cell survival measured by CTG after infection with different MOIs of MHV-A59. Mean±SD of triplicate wells per experiment. On the right, WB demonstrating efficient though incomplete LSD1 knockdown

C. Expression of IFNα and selected ISGs by qRT-PCR in response to MHV-A59 MOI 0.1 24 hpi and DMSO or DDP at 2.5 or 10 μM. Values are expressed as fold change over Time 0. Mean±SD of triplicate wells

D. No effect of JAK inhibitor I on DDP rescue of cell viability of infected L929 cells at different MOI as indicated. Mean±SD of triplicate wells per experiment

E. Expression of the lysosomal ATPase in BMDM (RPKM from RNAseq in FIG. 4)

F. Lysosome pH measurement using lysosensor on BMDM uninfected or infected with MHV-A59 MOI 5 and treated with DMSO or DDP 10 μM, at 12 hpi

G. BiP extrusion upon infection on BMDM uninfected or infected with MHV-A59 MOI 5 and treated with DMSO or DDP 10 μM, at 12 hpi, measured by WB on cell pellets and 1000 μl supernatant).

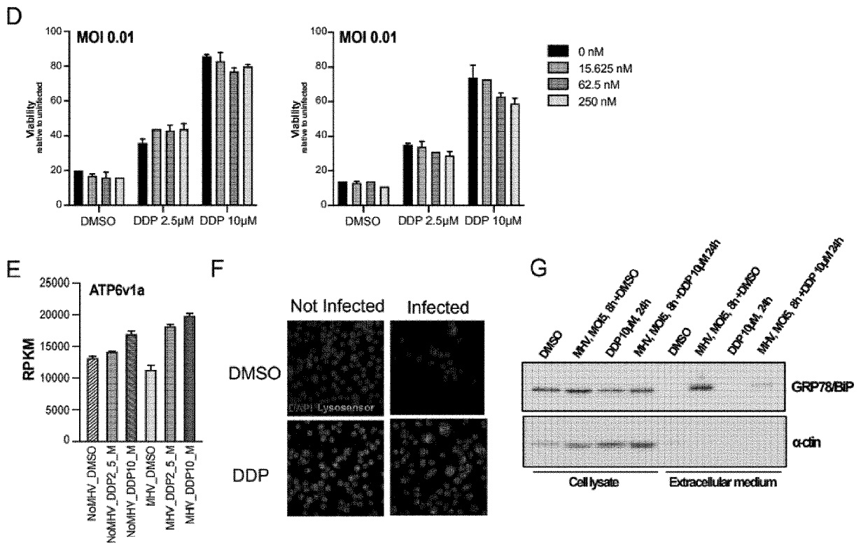

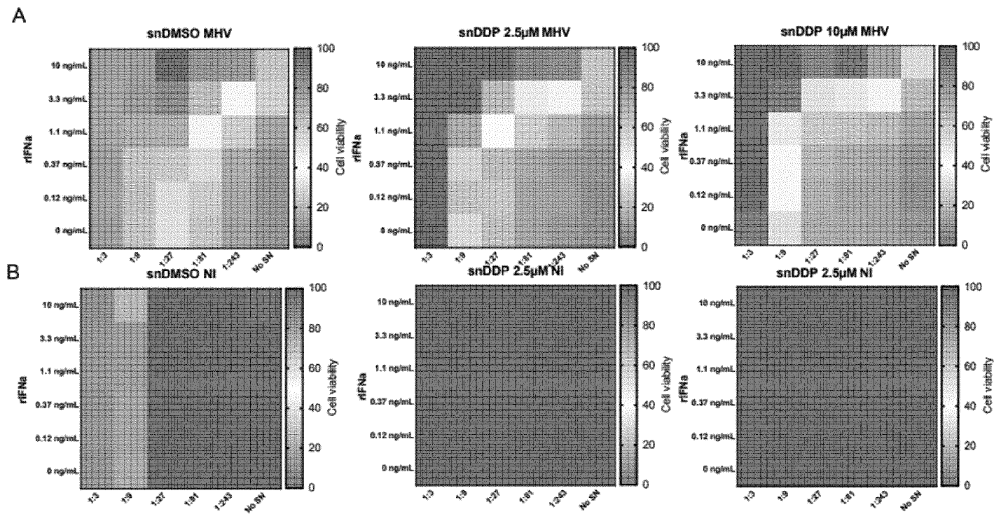

FIG. 15

A. Cell viability by CTG (relative to uninfected) of infected L929 cells co-treated with IFNα and supernatant of infected BMDMs treated with DMSO or DDP 2.5 or 10 μM

B. Cell viability by CTG (relative to uninfected) of uninfected L929 cells co-treated with IFNα and supernatant of infected BMDMs treated with DMSO or DDP 2.5 or 10 μM

C. Cell viability by CTG (relative to uninfected) of infected L929 cells co-treated with IFNα and DMSO or DDP 2.5 or 10 μM

D. Viral titer in human cells (CD14-derived macrophages, Calu3 and VeroE6) in response to in vitro inoculation of SARS-CoV2 MOI 0.1, showing lack of productive infection in macrophages

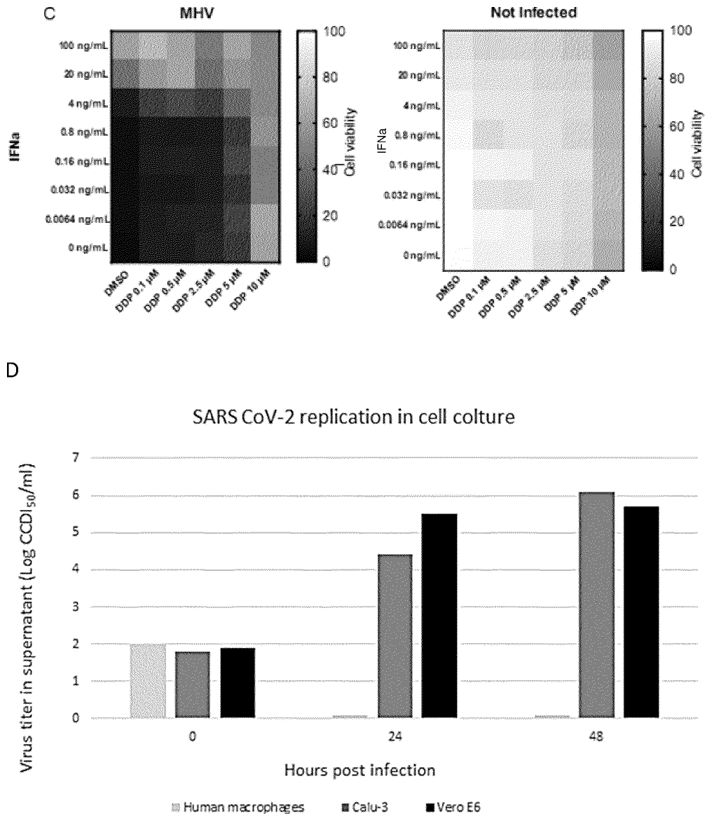

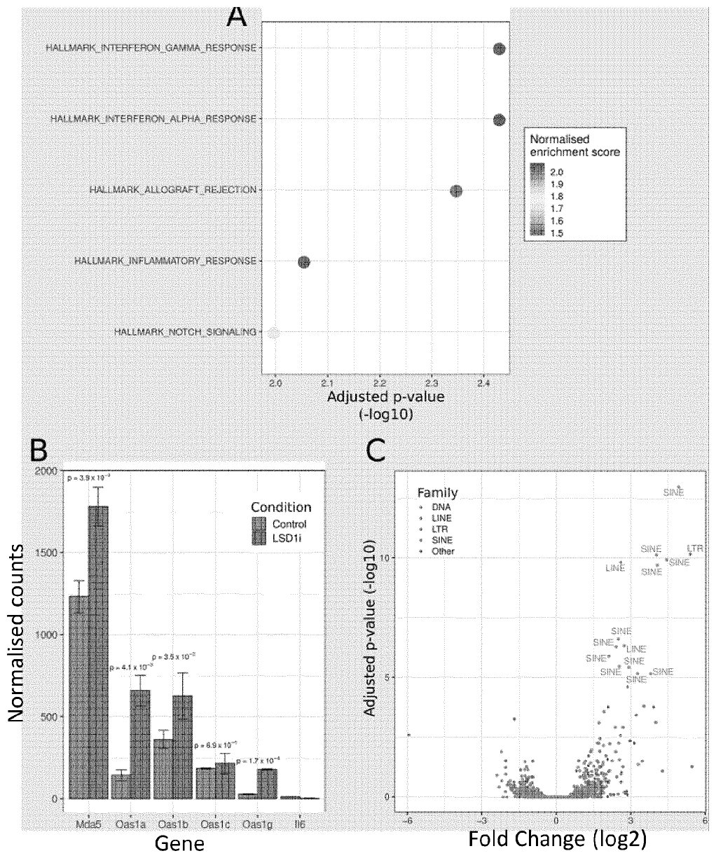

FIG. 16: In vivo effect of the treatment of hematopoietic myeloid leukemia cells using the LSD1 inhibitor 38003 (3 days) on the Interferon signaling (A), the expression of mouse ERV families (C) and of the dsRNA sensor OAS1 (RNAseq) (B).

### Materials and Methods

### Viral Stock.

MI-IV strain A59, was kindly provided by dr Riccardo Villa at the IZSLER and propagated on L929 cells. Briefly, L929 cells were plated the day before infection at a density of 20 million cells in T175 flask. Cells were infected at MOI: 0.5 in 10 ml of free serum DMEM. After 1 hour incubation at 37° C. 40 ml of DMEM supplemented with 3% FBS were added. Supernatant containing virus was harvested when the virus-induced cytopathic effect was visible on more than 70% of cells, usually 36 hours after infection. The identity of the virus was confirmed by Illumina sequencing (see Table 1a-c)

TABLE 1a

| strain | genbank acc code | link |
|---|---|---|
| A59 | Accession: | https://www.ncbi.nlm.nih.gov/ |
| | NC_048217.1 | nuccore/NC_048217.1 |
| | GI: 1842094262 | |
| Mhv-1 | ACCESSION | https://www.ncbi.nlm.nih.gov/ |
| | FJ647223 | nuccore/225403250 |
| | VERSION | |
| | FJ647223.1 | |
| Mhv-3 | GenBank: FJ647224.1 | https://www.ncbi.nlm.nih.gov/ |
| | | nuccore/FJ647224.1 |
| Mhv-jhm | AC_000192.1 | https://www.ncbi.nlm.nih.gov/ |
| | | nuccore/AC_000192.1 |

TABLE 1b

| | | | | indels |
|---|---|---|---|---|
| strain | genome coverage (%) | mismatches | indels | length |
| A59 | 99.962 | 36 | 1 | 3 |
| MHV-1 | 58.01 | 981 | 2 | 4 |
| MHV-3 | 7.117 | 75 | 0 | 0 |
| JHM | 48.16 | 627 | 0 | 0 |

TABLE 1c

| A59 | | | | | |
|---|---|---|---|---|---|
| reference | variants list | | | | |
| assembly code | our assembly | ref pos | ref | alt | alt pos |
| NC_048217.1 | NODE_1_length_31349_cov_10497.166922_g0_i0 | 417 | A | G | 407 |
| NC_048217.1 | NODE_1_length_31349_cov_10497.166922_g0_i0 | 818 | C | T | 808 |
| NC_048217.1 | NODE_1_length_31349_cov_10497.166922_g0_i0 | 3476 | G | A | 3466 |
| NC_048217.1 | NODE_1_length_31349_cov_10497.166922_g0_i0 | 4547 | C | T | 4537 |
| NC_048217.1 | NODE_1_length_31349_cov_10497.166922_g0_i0 | 4567 | T | C | 4557 |
| NC_048217.1 | NODE_1_length_31349_cov_10497.166922_g0_i0 | 5522 | T | C | 5512 |
| NC_048217.1 | NODE_1_length_31349_cov_10497.166922_g0_i0 | 6266 | T | C | 6256 |
| NC_048217.1 | NODE_1_length_31349_cov_10497.166922_g0_i0 | 6414 | C | T | 6404 |
| NC_048217.1 | NODE_1_length_31349_cov_10497.166922_g0_i0 | 7476 | A | G | 7466 |
| NC_048217.1 | NODE_1_length_31349_cov_10497.166922_g0_i0 | 7625 | G | C | 7615 |
| NC_048217.1 | NODE_1_length_31349_cov_10497.166922_g0_i0 | 9060 | A | G | 9050 |
| NC_048217.1 | NODE_1_length_31349_cov_10497.166922_g0_i0 | 9246 | T | C | 9236 |
| NC_048217.1 | NODE_1_length_31349_cov_10497.166922_g0_i0 | 9433 | C | A | 9423 |
| NC_048217.1 | NODE_1_length_31349_cov_10497.166922_g0_i0 | 10846 | T | C | 10836 |
| NC_048217.1 | NODE_1_length_31349_cov_10497.166922_g0_i0 | 11165 | C | T | 11155 |
| NC_048217.1 | NODE_1_length_31349_cov_10497.166922_g0_i0 | 11358 | C | T | 11348 |
| NC_048217.1 | NODE_1_length_31349_cov_10497.166922_g0_i0 | 12141 | A | G | 12131 |
| NC_048217.1 | NODE_1_length_31349_cov_10497.166922_g0_i0 | 12181 | A | G | 12171 |
| NC_048217.1 | NODE_1_length_31349_cov_10497.166922_g0_i0 | 12986 | A | G | 12976 |
| NC_048217.1 | NODE_1_length_31349_cov_10497.166922_g0_i0 | 13605 | A | G | 13595 |
| NC_048217.1 | NODE_1_length_31349_cov_10497.166922_g0_i0 | 18647 | C | A | 18637 |
| NC_048217.1 | NODE_1_length_31349_cov_10497.166922_g0_i0 | 20411 | T | C | 20401 |
| NC_048217.1 | NODE_1_length_31349_cov_10497.166922_g0_i0 | 22740 | G | T | 22730 |
| NC_048217.1 | NODE_1_length_31349_cov_10497.166922_g0_i0 | 22743 | C | T | 22733 |
| NC_048217.1 | NODE_1_length_31349_cov_10497.166922_g0_i0 | 23199 | AGA | . | 23189 |
| NC_048217.1 | NODE_1_length_31349_cov_10497.166922_g0_io | 23394 | T | C | 23381 |
| NC_048217.1 | NODE_1_length_31349_cov_10497.166922_g0_i0 | 23861 | C | T | 23848 |
| NC_048217.1 | NODE_1_length_31349_cov_10497.166922_g0_i0 | 24448 | A | G | 24435 |
| NC_048217.1 | NODE_1_length_31349_cov_10497.166922_g0_i0 | 24449 | T | C | 24436 |
| NC_048217.1 | NODE_1_length_31349_cov_10497.166922_g0_i0 | 24451 | A | T | 24438 |
| NC_048217.1 | NODE_1_length_31349_cov_10497.166922_g0_i0 | 26444 | C | A | 26431 |
| NC_048217.1 | NODE_1_length_31349_cov_10497.166922_g0_i0 | 28923 | A | G | 28910 |
| NC_048217.1 | NODE_1_length_31349_cov_10497.166922_g0_i0 | 28936 | T | C | 28923 |
| NC_048217.1 | NODE_1_length_31349_cov_10497.166922_g0_i0 | 29013 | C | G | 29000 |
| NC_048217.1 | NODE_1_length_31349_cov_10497.166922_g0_i0 | 29651 | C | T | 29638 |
| NC_048217.1 | NODE_1_length_31349_cov_10497.166922_g0_i0 | 30110 | T | A | 30097 |
| NC_048217.1 | NODE_1_length_31349_cov_10497.166922_g0_i0 | 30277 | C | T | 30264 |

For SARS-CoV2 experiments, a strain isolated from an Italian patient in February 2020 (Stefanelli et al., Euro Surveill. 2020 April; 25(13)) was used in the Biosafety level 3 facility of the Istituto Superiore di Sanita' in Rome.

The virus was propagated in Vero E6 cells that were propagated at 37° C. in 5% CO₂ in minimal essential medium (MEM) supplemented with 10% fetal calf serum (FCS), 1% 1-glutamine, and 1.4% sodium bicarbonate. Virus-infected cells were maintained at 37° C. in 5% CO₂ in MEM supplemented with 2% FCS. Titers were measured by CCID₅₀ system in Vero E6 cell. Briefly, samples were serially diluted 1/10 in medium. Then 100 μL of each dilution was plated into ten wells of 96-well plates containing 80-90% confluent cells. The plates were incubated at 37° C. under 5% carbon dioxide for five days. Each well was then scored for the presence or absence of the virus. The limiting dilution end point (CCID₅₀/ml) was determined by the Kärber equation

### Key Resource Table

Table below (Table 2) provides a list of materials used in the course of the present invention.

TABLE 2
Key resources table

| | | |
|---|---|---|
| REAGENT or RESOURCE | SOURCE | IDENTIFIER |

Antibodies

| | | |
|---|---|---|
| Rabbit Polyclonal Anti-LSD1 | Abcam | Cat#ab17721, |
| | | RRID: AB_443964 |
| Mouse Anti-Vinculin Monoclonal Antibody, Clone | Sigma-Aldrich | Cat#V9131, RRID: |
| hVIN-1 | | AB_477629 |
| Mouse Anti-TNF alpha Monoclonal Antibody (MP6- | Thermo Fisher | Cat#16-7321-81, |
| XT22), Functional Grade | Scientific | RRID: AB_494133 |
| Rat IgG1 kappa Isotype Control (eBRG1), | Thermo Fisher | Cat#14-4301-81, |
| eBioscience ™ antibody | Scientific | RRID: AB_470101 |
| InVivoMab anti-mouse IFNAR-1 antibody | Bio X Cell | Cat#BE0241, RRID: |
| | | AB_2687723 |
| InVivoMab mouse IgG1 isotype control antibody | Bio X Cell | Cat#BE0083, RRID: |
| | | AB_1107784 |
| Human anti-human TNF alpha monoclonal antibody | Miltenyi | Cat# 130-120-490, |
| (clone cA2), FITC conjugated | | RRID: AB_2752116 |
| Mouse anti-human CD11B monoclonal antibody | BD Biosciences | Cat# 560914 |
| (clone ICRF44), APC-Cy7 conjugated | | |
| Mouse anti-human CD80 monoclonal antibody | R&D Systems | Cat# FAB140P |
| (clone37711), PE conjugated | | |
| Human anti-human CD14 monoclonal antibody | Miltenyi | Cat #170-081-072 |
| (clone REA599), APC conjugated | | |
| Human anti-mouse TNF alpha monoclonal antibody | Miltentyi | Cat# 130-119-561 |
| (cloneREA636), PE conjugated | | |
| Mouse anti-mouse CD14 monoclonal antibody (clone | Invitrogen | Cat# 11-0141-82, |
| Sa2-8), FITC conjugated | | RRID: AB_464949 |
| Rat anti-mouse CD11B monoclonal antibody (clone | BD Biosciences | Cat# 553312 |
| M1/70), APC conjugated | | |
| Hamster anti-mouse CD80 monoclonal antibody | BD Biosciences | Cat# 561955 |
| (clone 16-10A1), PE conjugated | | |
| NF-κB p65 (D14E12) XP ® Rabbit mAb | Cell Signaling | Cat# 8242, RRID: |
| | Technology | AB_10859369 |
| Mouse Anti-NSP9 (Strain A59) monoclonal antibody | OriGene | Cat# AM08450PU-N |
| (clone 2C6.H1) | | |
| Rabbit Anti-dsRNA (clone J2) | CliniScience | Cat# Ab01299-23.0 |
| Mouse Anti-dsRNA (clone J2) | Scicons | Cat# 10010200 |
| Alexa Fluor ® 488 AffiniPure Donkey Anti-Mouse IgG | Jackson | Cat# 715-545-150 |
| (H + L) | ImmunoResearch | |
| Alexa Fluor ® 488 AffiniPure Donkey Anti-Rabbit IgG | Jackson | Cat# 715-545-152 |
| (H + L) | ImmunoResearch | |
| Alexa Fluor ® 647 AffiniPure Donkey Anti-Mouse IgG | Jackson | Cat# 715-605-150 |
| (H + L) | ImmunoResearch | |
| Cy ™3 AffiniPure Donkey Anti-Mouse IgG (H + L) | Jackson | Cat# 715-165-150 |
| | ImmunoResearch | |
| IRF Family Antibody Sampler Kit | Cell Signaling | Cat# 57103 |
| | Technology | |
| IRF-9 (D9I5H) Rabbit mAb (Mouse Specific) | Cell Signaling | Cat# 28845 |
| | Technology | |
| BiP (C50B12) Rabbit mAb | Cell Signaling | Cat# 3177, |
| | Technology | RRID: AB_2119845 |
| Anti-Lamin B1 antibody - Nuclear Envelope Marker | Abcam | Cat# ab16048, |
| | | RRID: AB_10107828 |
| Anti-alpha Tubulin antibody - Loading Control | Abcam | Cat# ab4074, |
| | | RRID: AB_2288001 |
| Bacterial and Virus Strains | | |
| Murine hepatitis virus (strain A59) | Dr. Riccardo Villa | NCBI: txid11142 |
| | IZSLER (Brescia) | |
| Biological Samples | | |
| Human Peripheral Blood from healthy donors | Blood Transfusion | N/A |
| | Service and | |
| | Hematology | |
| | Department of | |
| | Umberto I Hospital | |
| | (Rome, Italy) | |
| Murine Primary Bone Marrow Derived Macrophages | This study | N/A |
| from C57BL/6 strain | | |
| Chemicals, Peptides, and Recombinant Proteins | | |
| DDP-38003 LSD1 Inhibitor | Vianello et al. 2016 | CAS: 1831167-98-6 |
| ORY-1001 LSD1 Inhibitor | Vianello et al. 2016 | CAS: 1431326-61-2 |
| Dexamethasone | Sigma-Aldrich | Cat#D8893; CAS: |
| | | 50-02-2 |
| InSolution JAK Inhibitor I | Merck Millipore | Cat#420097; CAS: |
| | | 457081-03-7 |
| Recombinant Mouse M-CSF | Bio-techne Srl | Cat#416-ML-050 |
| Recombinant Human M-CSF | Peprotech | Cat#300-25 |
| LPS, from E coli O55: B5 | Merck Millipore | Cat#L4524 |
| R848 (Resiquimod) | InvivoGen | Cat#tlrl-r848; CAS: |
| | | 144875-48-9 (free |
| | | base) |
| Poly {I} Poly {C} | Cytiva | Cat #27473201 |
| Lipofectamine 2000 | Thermo Fisher | Cat#11668019 |
| | Scientific | |
| Recombinant Human TNF alpha | LSBio | Cat # LS-G259-50 |
| Recombinant Human IL-1 beta | R&D Systems | Cat # 210-LB |
| Recombinant Human IFN-gamma | Peprotech | Cat# 300-02 |
| Recombinant Human IL-4 | Peprotech | Cat# AF200-04 |
| Recombinant Human IL-13 | Peprotech | Cat# AF200-13 |
| Recombinant Mouse IL-4 | Merck Millipore | Cat# SRP3211 |
| Recombinant Mouse IL-13 | Peprotech | Cat# 200-13 |
| Recombinant Human Interferon alpha1 | Cell Signaling | Cat#8927SC |
| | Technology | |
| Recombinant mouse IFN gamma | R&D Systems | Cat#485-MI |
| Critical Commercial Assays | | |
| CellTiter-Glo ® Luminescent Cell Viability Assay | Promega | Cat#G7571 |
| Luna ® Universal One-Step RT-qPCR Kit | New England Biolabs | Cat#E3005L |
| Direct-zol RNA Miniprep Kit | Zymo Research | Cat#R2050 |
| eBioscience ™ Intracellular Fixation & | Thermo Fisher | Cat#88-8824-00 |
| Permeabilization Buffer Set | Scientific | |
| Murine TNF alpha ELISA Set II | BD Biosciences | Cat#558534 |
| Human TNF alpha Quantikine ELISA kit | R&D Systems | Cat# DTD00D |
| Murine pan-Interferon alpha Verikine ELISA kit | PBL Assay Sciences | Cat# 42115-1 |
| Human pan-Interferon alpha Verikine ELISA kit | PBL Assay Sciences | Cat# 41115-1 |
| Murine Interferon beta Verikine ELISA kit | PBL Assay Sciences | Cat# 42400-1 |
| Murine Interferon gamma ELISA kit | Sigma Aldrich | Cat# RAB0224 |
| Human Interferon gamma ELISA kit | Sigma Aldrich | Cat# RAB0222 |
| Human CD14 microbeads | Miltenyi | Cat# 130-050-201 |
| Dynabeads ™ Protein G for Immunoprecipitation | Thermo Fisher | Cat# 10004D |
| | Scientific | |
| Agencourt AMPure XP beads | Beckman Coulter | Cat# A63880 |
| Fast SYBR ™ Green Master Mix - Thermo Fisher | Thermo Fisher | Cat# 4385612 |
| Scientific | Scientific | |
| QIAquick PCR Purification Kit | QIAGEN | Cat# 28106 |
| LysoSensor ™ Green DND-189 - Special Packaging | Thermo Fisher | Cat# L7535 |
| | Scientific | |
| Experimental Models: Cell Lines | | |
| Murine: NCTC clone 929 | DSMZ | Cat# ACC-2, RRID: |
| | | CVCL_0462 |
| Murine: LA-4 | ATCC | Cat# CCL-196, |
| | | RRID: CVCL_3535 |
| Human: CALU-3 | ATCC | Cat# HTB-55, RRID: |
| | | CVCL_0609 |
| Human: Vero-E6 | ATCC | Cat#CRL-1586 |
| Experimental Models: Organisms/Strains | | |
| Mouse: C57BL/6 | Charles River | RRID: |
| | | IMSR_JAX: 00064 |
| Oligonucleotides | | |
| shRNA scramble sequence: | | N/A |
| 5′-GTGGACTCTTGAAAGTACTAT-3′ (SEQ ID No. 1) | | |
| shRNA Lsd1 targeting sequence: | Broad Institute-GPP | TRCN0000071375 |
| 5′-CCACAAGTCAAACCTTTATTT-3′ (SEQ ID No 2) | Web Portal | |
| CRISPR oligo Lsd1 targeting sequence: | Sheng et al., 2018 | N/A |
| CCTGAGAGGTCATTCGGTCA (SEQ ID No. 3) | | |
| Recombinant DNA | | |
| pLKO.1 | Stewart et al. 2003 | RRID |
| | | Addgene_8453 |
| pSpCas9(BB)-2A-GFP | Ran et al., 2013 | Addgene Cat#48138 |
| pWPXL-TTT-H2B-GFP | Falkowska-Hansen et | N/A |
| | al., 2010 | |

Software and Algorithms

| | | |
|---|---|---|
| ImageJ | Schneider et al., 2012 | https://imagej.nih.gov/ |
| | | ij/ |
| Bedtools | Quinlan et al., 2010 | v2.28.0 |
| | | https://github.com/arq5x/ |
| | | bedtools2 |
| Markov Cluster Algorithm | Enright et al., 2002 | v14-137 |
| | | https://micans.org/mcl/ |
| SPAdes | Nurk, Bankevich et al., | https://anaconda.org/ |
| | 2012 | bioconda/spades |
| QUAST | Gurevich et al., 2013 | http://quast.sourceforge.net/ |
| STAR | Dobin et al., 2013 | https://github.com/ |
| | | alexdobin/STAR |
| Samtools | Li et al., 2009 | https://github.com/ |
| | | samtools/samtools |
| Htseq | Anders et al., 2014 | https://github.com/ |
| | | simon-anders/htseq |
| Galaxy | Afgan et al., 2016 | https://usegalaxy.org/ |
| DESeq2 | Love et al., 2014 | v1.28.0 |
| | | https://bioconductor.org/ |
| | | packages/release/ |
| | | bioc/html/DESeq2.html |
| RepeatMasker | Smit, AFA, Hubley, R | v405 |
| | & Green, P. | https://www.repeatmasker.org/ |
| | RepeatMasker Open- | |
| | 4.0. | |
| | 2013-2015 | |
| | <http://www.repeatmasker.org>. | |

### Cell Culture

Raw264.7, L929, LA4, Calu3 and VeroE6 cell lines were purchased by American Type Culture Collection and grown according to ATCC recommendations. Cultures were maintained in a humidified tissue culture incubator at 37° C. in 5% CO₂. To assure mycoplasma-free conditions, all cells were routinely tested.

BMDM were obtained from bone marrow of 6-10 week old female C57B16 mice (Charles River). 106 cells were plated in 10 cm untreated cell culture dishes, resuspended in 8 ml of Alpha MEM containing 20% FBS, 2 mM of L-glutamine, antibiotics, 40 ng/ml of rm M-CSF (R&D System) and allowed to differentiate for 7 days.

Human monocytes were purified from peripheral blood collected from healthy blood donors at the Centro Trasfusionale Policlinico Umberto I, University La Sapienza blood bank (Rome, Italy) using Ficoll gradients (lymphocyte-H; Cedarlane). CD14 cells were purified by anti-CD14 monoclonal antibody (mAb)-conjugated magnetic microbeads (Miltenyi Biotec) and were cultured for 6 days in RPMI 1640 medium (Life Technologies Invitrogen), supplemented with heat-inactivated 10% lipopolysaccharide-free FBS, 1 mM sodium pyruvate, 0.1 mM nonessential amino acids, 2 mM L-glutamine, 25 mM HEPES, 100 U/ml penicillin, 100 μg/ml streptomycin (all from EuroClone) in the presence of human recombinant M-CSF (100 ng/ul; Peprotech). Blood donors provided written informed consent for the collection of samples and subsequent analysis. Blood samples were processed anonymously.

### Generation of H2^{GFP}-Expressing Cells

L929 cells were plated at a density of 5×104 in 24 well plate a day before the infection. A spin infection was used to infect L929 cells using a lentiviral vector carrying the H2B-GFP transgene (Falkowska-Hansen et al., 2010). Briefly, concentrated H2B-GFP lentivirus (MOI 4) was added to L929 cell in a 24-well non-tissue culture-treated plate and centrifuge at 750×g, 25° C. for 1 h. Infected cells were incubated for 3 h at 37° C. and replaced with fresh culture media (1 mL/well). After a recovery period of 2 days, GFP+ cells were sorted by fluorescence-activated cell sorting (FACS) and maintained in the culture for further experiments.

### In Vitro Pharmacological Treatments:

DDP38003 and Oryzon 1001 were synthesized as described in Journal of Medicinal Chemistry (2016), 59(4), 1501-1517.

Dexamethasone (Sigma) was dissolved in DMSO.

polyIC was purchased from Cytiva and dissolved in PBS to a concentration of 1 mg/ml.

LPS was purchased from Sigma and dissolved in water to a concentration of 1 mg/ml.

### Preparation of BMDM Supernatant

BMDM were plated at a density of 500.000 cells per well in untreated cell culture 6 well plates, in a total volume of 2 ml. After one night incubation, BMDM were treated with the indicated compounds. 24 hours later the cells were infected with MI-IV at the desired MOI (Multiplicity of Infection), by replacing the overnight medium with DMEM 3% FCS (Fetal Cow Serum) containing MI-IV. After 1 hour, the viral inoculum was removed and replaced with BMDM medium supplemented with the drugs. 24 hours later, supernatant was harvested, clarified by centrifugation and used for the subsequent experiments.

### UV Inactivation of BMDM Supernatant

500ul of BMDM supernatant were aliquoted in one 24 well and incubated on ice for 1 minute. UV inactivation was performed on ice, using Agilent Genomics/Stratagene Stratalinker 2400 UV Crosslinker, by delivering an energy dose equivalent to 0.3 Joules.

### Antiviral and Cytotoxicity Assay

L929 cells were plated at a density of 5000/well in 96 well plate, in a total volume of 100ul. The day after, the culture medium was removed and replaced with 50ul of the serially diluted UV inactivated BMDM supernatant. After 1 hours incubation—to test BMDM supernatant antiviral activity—cells were infected by adding of 50ul of DMEM supplemented with 3% FCS containing the desired MOI, or left uninfected—to test the BMDM supernatant cytotoxic effect—by adding 50ul of DMEM supplemented with 3% FCS without virus. 48 hours later, the vitality of the cells was evaluated by CellTiter-Glo luminescent cell viability assays (Promega, Madison, WI, USA), following the manufacturer's instructions.

### Viral Titration (TCID50)

Titration for MHV and SARS-CoV2 was performed using the TCID50 method, with some specifications. Briefly, supernatants from infected cells were 10-fold serially diluted and titrated on target cells plated at 80-90% confluence (for MHV, L929 cells; for SARS-CoV2, VeroE6 cells) in 96 wells in a total volume of 100 μl, (8 replicate wells for MHV, 10 replicate wells for SARS-CoV2) and incubated at 37° C. under 5% carbon dioxide. After a defined time period (2 days for MHV, 5 days for SARS-CoV2), each well was scored for the presence of virus-induced cytopathic effects. The limiting dilution end point (TCID50) was determined using the Reed-Muench method for MHV and the Kärber equation for SARS-CoV2.

### Generation of LSD1 KO Cells by CRISPR/Cas9.

The sgRNA oligo (see Table 2; Sheng et al., 2018), targeting exon 3 of Lsd1, was cloned into pSpCas9(BB)-2A-GFP, a gift from Feng Zhang (Addgene plasmid #48138; http://n2t.net/addgene:48138; RRID:Addgene 48138) (Ran et al., 2013). The plasmid was subsequently transfected, in parallel with empty vector controls, in L929 cells using Lipofectamine 2000 according to manufacturer's instructions. Forty-eight hours later, GFP-positive cells were single-cell sorted in 96-well plates and after clonal expansion, sublines were screened by Western blot against LSD1.

### RNAi Knockdown

To knock down LSD1, one short hairpin RNA (shRNA) sequences was tested. shLSD1 #1 (see Table 2 for sequence) was cloned into the pLKO vector by AgeI-Eco RI double digestion. The plasmid was used to produce lentiviral particles in 293T cells.

### Size Exclusion Chromatography

5 ml of UV-inactivated cell culture supernatants were fractionated into 53 fractions on a Superdex 200 16/60 column (Cytiva Life Sciences) with PBS as eluting buffer. 1 ml fractions were collected and analyzed by antiviral and cytotoxic assays, and ELISA.

### Live Cell Imaging

L929 (20 k cells), BMDM (5K or 10 k cells), BMDM:L929 1:1 (12.5 k:12.5 k cells) and BMDM:L929 2:1 (20 k:10 k cells) cocultures were seeded on a 96-wells plate at day 0, treated at day 1, infected with MHV 0.1MOI at day 2 for 1 h, washed and kept in fresh medium plus treatments and 0.4 ug/m1Propidium Iodide (PI) for the total duration of the time-lapse experiment. GFP, PI and bright field images were acquired on a Nikon Eclipse Ti microscope (Nikon Instruments S.p.A., Firenze, Italy) equipped with a xyz motorized stage, a Spectra X light engine (Lumencor, Beaverton, OR, USA), a Zyla 4.2 sCMOS camera (Andor, Oxford Instruments plc, Tubney Woods, Abingdon, Oxon OX13 5QX, UK) a multi-dichroic mirror and single emission filters (Semrock, Rochester, New York, USA). Large images made by four partially overlapping (2%) fields of view (FOV) were acquired for each well with a 2×2 camera binning every hour from 4 hpi to 48 hpi using a 10×, 0.3 NA objective lens. Temperature, CO₂ and humidity was controlled by a microscope cage incubator (Okolab, Napoli, Italy).

Bone marrow derived macrophages or L929 cells were seeded on a 12 glass-bottom wells (MatTek Corporation, Ashland, MA 01721, USA), coated with poly-D-Lysine 0.1% (w/v) in water (3×105 cells/well). After 24 hours cells were treated with DDP (10 μM) or DMSO and then infected with MHV for 12 hours. Cells were incubated with 1 μM of Lysosensor Green DND-189 (Thermo Fisher Scientific, Monza, Italy) for 90 min. and Hoechst 33342 (Euroclone S.p.A., Pero, Italy) for the last 30 min. Cells were washed and fresh medium was added. Labelled live cells were imaged at 37° C. and 5% CO₂ on a Leica Thunder Imager system (Leica Microsystems GmbH, Wetzlar, Germany), equipped with a xy motorized stage, 5 LED sources, a DFC9000 GTC sCMOS camera, a multi-dichroic mirror and 4 emission filters. Ninety-nine images were acquired for each condition using a 63×1.4NA oil immersion objective lens.

### Immunofluorescence

Cells were seeded on Poly-D-lysine coated slides (10⁵ cells/slide). After treatments cells were fixed with methanol at −20° C. for 6 min, blocked with 5% donkey serum for 60 min. Slides were stained with primary antibodies diluted in 1% BSA in PBS (NF-kB and IRF-1 1:400, NSP9 1:1000) for 90 min. Secondary anti-rabbit (A488) and anti-mouse (Cy3) were used at 1:200 for 1 hour. Nuclei were stained with DAPI 1:1000 for 20 min. Mowiol was used as mounting solution. Cells labelled with DAPI, with the anti-NFkB or anti-IRF1 primary Ab and AlexaFluor488 conjugated secondary Ab and anti-NSP9 primary Ab and Cy3 conjugated secondary Ab were imaged with a 60×1.4 NA oil immersion objective lens on a CSU-W1 Yokogawa Spinning Disk confocal system with a 50 μm pinhole disk mounted on an Eclipse Ti2 stative and equipped with a motorized xyz stage, 6 solid state lasers, a multi-dichroic mirror, single emission filters and a Prime BSI sCMOS camera (TELEDYNE PHOTOMETRICS, Tucson, AZ 85706, USA). Hundred FOV per condition were automatically acquired thanks to the JOBS application of the NIS software (Nikon). Briefly, for each of the 100 positions defined in the JOB, an autofocus routine using the DAPI channel was used to define the acquisition focal plane and the 3 channels corresponding to the 3 stainings were acquired.

### Bioinformatic Analysis of Imaging Data:

### Syncytia and Death Events Quantification

Time-lapse raw images were first corrected for uneven illumination (shading correction) and background (BG) variation in time thanks to the BaSiC Fiji/Image) plugin (Peng, T. et al, 2017). The corrections were done in batch using a custom made Image) macro. Briefly, for each 4-channel .nd2 time series, the channels were split, BaSiC-corrected and saved as tiff sequence in a new folder.

A custom-made Image) macro was used to identify syncytia formed by infected L929^{GFP} cells. Briefly, for each condition and in batch, the GFP time series was BG-corrected using a rolling ball radius of 50 pixels, filtered with a median filter (radius=2 pixels), then objects were identified using a threshold defined by the Otsu method, the objects with a size bigger than 10 um² were identified by the Analyze Particles Image) function and the region of interest (ROI) added to the ROI manager. In each Field of View (FOV), the list of ROI was used to calculate the ROI area and the intensity of the PI channel.

The mean area of single nuclei was calculated from the control condition (DMSO, not infected) at the first time point and the mean nuclei area resulted about 150 um². Syncytia were arbitrarily considered as the union of at least 5 nuclei, using an object size threshold of 750 um². This threshold was visually confirmed to accurately capture the majority of syncitia.

To calculate the death events of L929 cells, the mean PI intensity in each ROI obtained from the segmentation of the GFP channel was considered, and an intensity threshold of 2000 grey levels was used to define a PI-positive object. For both populations, the number of objects were transformed in nuclei number (“death events”) multiplying every single object by a integer factor (>=1) calculated dividing the area of each identified object by the mean nucleus area (150 um²) and rounding to the minor integer. This correction was necessary to avoid underestimating death events associated with syncitia in-L929 infected cells. Live cells were calculated by dividing the total estimated nuclei by the number of death events, per each frame. In all figures, death events are expressed in absolute terms, whereas live cells are expressed relative to the number of live cells at the earliest recorded time frame (4 hpi).

### NFkB and IRF1 Immunofluorescence Quantification

Confocal images of BMDM were analyzed by a custom-made ImageJ macro written in Jython. Briefly, the DAPI channel, after a median filter and BG subtraction, was used to automatically segment the nuclei. To the nuclei binary image a Voronoi filter was applied to roughly identify the area relative to each cell. For each cell identified by a nucleus, a band around the nucleus with a thickness of 6 μm the cytoplasm was created and the intersection between the band and the cell area was considered as the “cytoplasm”. The total NSP9 intensity inside the cytoplasm was measured for each cell. The signal corresponding to 2 standard deviations above the mean NSP9 signal in the non-infected cells was used as threshold to identify the NSP9 positive and NSP9 negative cell populations in infected cells

The IRF1/NFKB nuclear signal was quantified inside the ROI obtained from the DAPI segmentation as mean intensity

### Lysosensor Assay

Bone marrow derived macrophages or L929 cells were seeded on a 12 glass-bottom wells (MatTek Corporation, Ashland, MA 01721, USA), coated with poly-D-Lysine 0.1% (w/v) in water (3×105 cells/well). After 24 hours cells were treated with DDP (10 μM) or DMSO and then infected with MHV for 12 hours. Cells were incubated with 1 μM of Lysosensor Green DND-189 (Thermo Fisher Scientific, Monza, Italy) for 90 min. and Hoechst 33342 (Euroclone S.p.A., Pero, Italy) for the last 30 min. Cells were washed and fresh medium was added. Labelled live cells were imaged at 37° C. and 5% CO2 on a Leica Thunder Imager system (Leica Microsystems GmbH, Wetzlar, Germany), equipped with a xy motorized stage, 5 LED sources, a DFC9000 GTC sCMOS camera, a multi-dichroic mirror and 4 emission filters. Ninety-nine images were acquired for each condition using a 63×1.4NA oil immersion objective lens. Widefield images of the DAPI and Lysosensor were quantified using a custom-made ImageJ macro. Similarly to what was done for the NSP9 quantification, the “cytoplasmic” Lysosensor total signal was calculated in a 6 μm thick band around the nucleus after BG subtraction.

### ChIP Assay.

Plates containing 15×106 cells were washed 3 times with PBS and fixed at RT with 1% formaldehyde for 15 min. Cells were washed again 3 times with PBS, harvested with a cell lifter, collected into Falcon tubes and centrifuged at 424 rcf for 5 min at 4° C. Each pellet was resuspended in 3 ml of Lysis Buffer 1 (50 mM Hepes-KOH, pH 7.5, 140 mM NaCl, 1 mM EDTA, 10% glycerol, 0.5% NP-40 and 0.25% Triton X-100) and incubated on ice for 10 minutes. Nuclei were pelleted at 1600 rcf for 5 min. at 4° C., washed with 3 ml of Lysis Buffer 2 (10 mM Tris-HCl, pH 8.0 5M, 200 mM NaCl, 1 mM EDTA and 0.5 mM EGTA) and incubated at RT for 10 minutes. Nuclei were pelleted again at 424 rcf for 5 minutes at 4° C. and the nuclear membrane was disrupted with 1.5 ml of Lysis Buffer 3 (10 mM Tris-HCl, pH 8.0, 100 mM NaCl, 1 mM EDTA, 0.5 mM EGTA, 0.1% Na-Deoxycholate, 0.5% N-lauroylsarcosine and protease inhibitors). Chromatin fragmentation was performed by sonication (Bioruptor® Plus sonication device, 45-60 cycles, 30 seconds on/off, high power, at 4° C.). Chromatin extracts containing DNA fragments with an average of 300 bp were then subjected to immunoprecipitation. The immunoprecipitation was performed using magnetic Dynabeads Protein G. Beads were blocked with 0.5% BSA in PBS and then mixed with the different antibodies (15 μg antibody:100 μl beads ratio) and incubated overnight on a rotating platform at 4° C. 1% of Triton X-100 was added to the sonicated lysates and lysates were centrifuged in microfuge (8000 g, 10 min. at 4 C) to pellet debris. Supernatants containing chromatin were subjected to immunoprecipitation and the 10% of the volume was used as input. Antibody-coated beads were added to each lysate and incubated overnight on a rotating platform at 4° C. Beads were washed 6 times (5 min each) with Wash Buffer (RIPA) and once with TE and 50 mM NaCl. The immunocomplexes were eluted in 100 μl of elution buffer (TE and 2% SDS) at 65° C. for 15 minutes. To reverse cross-links immunocomplexes (and inputs) were treated with RNAase (0.5 mg/ml) at 37° C., 20 min followed by proteinase K and 1% SDS at 65° C. overnight. DNA was purified using AMPure XP beads following manufacturer's instructions. Isolated DNA was used to analyze by quantitative PCR the expression of NF-κB targets (TNFα, IL1β, CXCL1, CCL5, CXCL10 and a negative control), with the Fast SYBR™ Green Master Mix on a thermocycler Viia7 (Life Technologies, Inc.)

### RT-qPCR

Total RNA was purified using the RNeasy kit (Qiagen). RT-qPCR was performed using Luna® Universal One-Step RT-qPCR Kit (New England Biolabs), following the manufacturer's instructions. Primers details are specified in table 3.

TABLE 3

| Gene | Organism | Forward | Reverse | Assay |
|---|---|---|---|---|
| Il1β | Mus | GACCTTCCAGGATGAGGACA (SEQ | TCCATTGAGGTGGAGAGCTT (SEQ | RT-qPCR |
| | musculus | ID NO. 4) | ID NO. 5) | |
| Il6 | Mus | CCATAGCTACCTGGAGTACATG(SEQ | TGGAAATTGGGGTAGGAAGGAC | RT-qPCR |
| | musculus | ID NO. 6) | (SEQ ID NO. 7) | |
| Tnfα | Mus | TCTTCTCATTCCTGCTTGTGG (SEQ | CACTTGGTGGTTTGCTACGA (SEQ | RT-qPCR |
| | musculus | ID NO. 8) | ID NO. 9) | |
| IFNα | Mus | TCTGATGCAGCAGGTGGG (SEQ ID | AGGGCTCTCCAGACTTCTGCTCTG | RT-qPCR⁽¹⁾ |
| | musculus | NO. 10) | (SEQ ID NO. 11) | |
| Isg15 | Mus | GGGGGAGAGTCGATCCAG (SEQ ID | CCCCAGCATCTTCACCTTTA (SEQ | RT-qPCR |
| | musculus | NO. 12) | ID NO. 13) | |
| Ifit1 | Mus | GCTCTGCTGAAAACCCAGAG (SEQ | CCCAATGGGTTCTTGATGTC (SEQ | RT-qPCR |
| | musculus | ID NO. 14) | ID NO. 15) | |
| Ifit2 | Mus | CACCTTCGGTATGGCAACTT (SEQ | GCAAGGCCTCAGAATCAGAC | RT-qPCR |
| | musculus | ID NO. 16) | (SEQ ID NO. 17) | |
| Oas1a | Mus | GGGGGAGAGTCGATCCAG (SEQ ID | CCCCAGCATCTTCACCTTTA (SEQ | RT-qPCR |
| | musculus | NO. 18) | ID NO. 19) | |
| Osl1 | Mus | CTCCGAGGTCTACGCAAATC (SEQ | CAGCTCCAGGGCATAGAGAG | RT-qPCR |
| | musculus | ID NO. 20) | (SEQ ID NO. 21) | |
| Mx2 | Mus | GACATTGCCACCACAGAGG (SEQ ID | CTGCTCTTGGATGTCCTGCT (SEQ | RT-qPCR |
| | musculus | NO. 22) | ID NO. 23) | |
| TNFα | Homo | GGCTCCAGGCGGTGCTTGTTC (SEQ | AGACGGCGATGCGGCTGATG | RT-qPCR |
| | sapiens | ID NO. 24) | (SEQ ID NO. 25) | |
| IL6 | Homo | CACTGGCAGAAAACAACCTGAA | ACCAGGCAAGTCTCCTCATTGA | RT-qPCR |
| | sapiens | (SEQ ID NO. 26) | (SEQ ID NO. 27) | |
| IFNα | Homo | TTGATGGCAACCAGTTCCAG (SEQ | TCATCCCAAGCAGCAGATGA (SEQ | RT-qPCR⁽²⁾ |
| | sapiens | ID NO. 28) | ID NO. 29) | |
| | | | TGTTCCCAAGCAGCAGATGA (SEQ | RT-qPCR⁽²⁾ |
| | | | ID NO. 30) | |
| ISG15 | Homo | CGCAGATCACCCAGAAGATCG (SEQ | TTCGTCGCATTTGTCCACCA (SEQ | RT-qPCR |
| | sapiens | ID NO. 31) | ID NO. 32) | |
| NEG2 | Mus | TTTTCCAGGCAAAGCAGATT (SEQ ID | ATGTATGGGCACAAGCACAA (SEQ | ChIP-qPCR |
| | musculus | NO. 33) | ID NO. 34) | |
| Cxcl1 | Mus | TAATCCTTGGGAGTGGAGCA (SEQ | GTGTGGCTGGAGTCTGGAGT | ChIP-qPCR |
| | musculus | ID NO. 35) | (SEQ ID NO. 36) | |
| Il1β | Mus | AATAATGCCCATTTCCACCA (SEQ ID | GCTGTGAAATTTTCCCTTGG (SEQ | ChIP-qPCR |
| | musculus | NO. 37) | ID NO. 38) | |
| Ccl5 | Mus | CTGTCCTGGCTTAGGCTTTG (SEQ | GGAAACCCCACGACTGACTA | ChIP-qPCR |
| | musculus | ID NO. 39) | (SEQ ID NO. 40) | |
| Cxcl10 | Mus | CCGGCTGCTGAGGAGTATTT (SEQ | AGCAATGCCCTCGGTTTACA (SEQ | ChIP-qPCR |
| | musculus | ID NO. 41) | ID NO. 42) | |
| Tnfα | Mus | CGCGGATCATGCTTTCTGTG (SEQ | GAAAAGCAAGCAGCCAACCA | ChIP-qPCR |
| | musculus | ID NO. 43) | (SEQ ID NO. 44) | |

⁽¹⁾Used in RT-qPCR experiments to assess the collective expression of murine interferon alpha genes.
⁽²⁾Used in RT-qPCR experiments to assess the collective expression of human interferon alpha genes.
All 3 primers were used in a single reaction.

### RNA Sequencing

mRNA-seq libraries were prepared according to the TruSeq low sample protocol (Illumina, San Diego, CA, USA), starting with 1 μg of total RNA per sample. RNA-seq libraries were pair-end sequenced on an Illumina NovaSeq 6000 sequencing platform. RNA-seq data were mapped using STAR aligner (Dobin et al., 2013) against the mouse genome (mm10). Counts were obtained by htseq-counts (Anders et al., 2015) and differential expression analysis was performed with DESeq2 package hosted in Galaxy online platform (Afgan et al., 2018) using a false discovery rate (FDR) cut-off of 1×10-4 (Kim et al., 2018). Hierarchical clustering was performed on z-score across samples for each gene, using Ward's criterion with 1−(correlation coefficient) as a distance measure.

Transposable Element Quantification from RNAseq

In order to quantify the expression of Transposable Elements (TEs) while avoiding the biases introduced by multimapping reads, we applied a clustering procedure that groups together TEs whose expression is supported by the same set of multimapping reads. Briefly, reads were mapped to the mouse reference genome (GRCm38 assembly) using Star and allowing an unlimited number of mapping locations. Then, BAM files for all samples were pooled and the coordinates of mapped reads were intersected (bedtools intersect; Bioinformatics, Volume 26, Issue 6, 15 Mar. 2010, Pages 841-842, https://doi.org/10.1093/bioinformatics/btq033) with those of TEs annotated in RepeatMasker (v405). This operation allowed us to build a binary matrix associating each read to the TEs that it maps to. Such matrix was then transformed with mcxarray into a square matrix of Tanimoto distances between each pair of TEs and subjected to clustering using the Markov Cluster Algorithm (MCL, filtering parameter >=0.5. Tanimoto distance. Inflation parameter 1.2; Nucleic Acids Research, Volume 30, Issue 7, 1 Apr. 2002, Pages 1575-1584, https://doi.org/10.1093/nar/30.7.1575). This operation generated 514.866 clusters that contain a variable number of TEs with common mappability profiles. The number of reads in each cluster was then quantified for each sample (discarding reads mapping to multiple clusters) and the resulting TE expression matrix was imported into R for differential expression analysis with DESeq2 (Genome Biology volume 15, Article number: 550 (2014); https://doi.org/10.1186/s13059-014-0550-8). Cluster counts for each sample were first normalised using size factors estimated from the number of reads uniquely mapping to Ensembl genes, then differential expression analysis was performed using the Wald Test and a design formula capturing the interaction between DDP treatment and MHV infection status (˜Infection*Treatment). The p-values thus obtained were then corrected for multiple hypothesis testing using the Benjamini-Hochberg procedure. To generate fold change boxplots for LINEs, SINEs and LTR clusters, we first discarded all clusters containing TEs belonging to multiple Repeat Masker classes. For the remaining the TEs, we then plotted the log 2 fold changes estimated by DESeq2 as boxplots. In order to test for differences in these distribution we used Welch's t test.

### Viral Genome Analysis and Assembly

To identify the MHV viral strain we used by the SPADES software (Nurk et al, 2013). The longest contig obtained was compared with the most common strains of MHV (MHV1, MHV3, JHM, A59-Table 1) using QUAST tool (Gurevich et al, 2013).

### Western Blotting

RIPA buffer, containing the cOmplete™ Protease Inhibitor Cocktail for all experiments, except for experiment in Supplementary FIG. 8A, in which 8M Urea buffer was used at RT. Cytoplasmic-nuclear protein fractions were performed as previously described (Czerkies et al., 2018). Cells were scraped in cold PBS, with a cell lifter, and centrifuged at 1350 rpm, 5 min at 4° C. Extracellular membranes were lysed, adding 1 ml of a hypotonic buffer (20 mM HEPES pH 8.0, 0.2% NP-40, 1 mM EDTA, 1 mM DTT and protease inhibitors) and incubating on ice for 10 min. The supernatant obtained after centrifugation at 1700×g, 5 min at 4° C. contained the cytoplasmic protein fraction; the pelleted nuclei were washed with the same buffer and centrifuged again. The nuclear content was extracted by adding 150 μl of nuclear fraction buffer (20 mM HEPES pH 8, 420 mM NaCl, 20% glycerol, 1 mM EDTA, 1 mM DTT and protease inhibitors), incubating on ice for 30 min and isolating the supernatant after centrifugation at 10,000×g, 10 min at 4° C. For both total and cytoplasmic-nuclear fractions, proteins were quantified using the Pierce™ BCA Protein Assay Kit (Thermo Fisher Scientific). Equal amount of proteins was mixed with Laemmli buffer and analyzed by SDS-PAGE, using nitrocellulose membranes. After blocking, membranes were incubated over night with the different primary antibodies at 4° C. and secondary antibodies for 1 hour at RT. Digital images were obtained using Clarity Western ECL Substrate and the ChemiDoc™ MP Imaging System (Bio-Rad)

### ELISA

To measure levels of cytokines secreted by BMDMs in response to MHV infection, ELISA was performed using 24 hrs post-infection supernatant from both infected and infected BMDM cultures. Murine TNFa levels were measured from 10 ul of supernatant using the Mouse TNFa ELISA Set II kit (BD Biosciences) while murine IFNa was measured from 100ul of supernatant using the Verikine-High Sensitivity mouse IFNa all subtypes ELISA kit (PBL Assay Science). ELISAs were performed in duplicate according to manufacturer's protocols. Similarly, TNFa and pan-IFNa were measured in fractions from size exclusion chromatography using 100ul of fraction volume.

### Mouse Leukemia Model.

Murine Acute Promyelocytic Leukemias (APL) were generated in mice genetically engineered to express the human PML/RARa fusion transcript (Westervelt et al, High-penetrance mouse model of acute promyelocytic leukemia with very low levels of PML-RARalpha expression, Blood. 2003 Sep. 1; 102(5):1857-65). Cells were obtained from leukemic spleens or bone marrows and propagated by intravenous injection into recipient wild-type C57/B16 mice. For the described experiments, 3^{rd} passage (P3) cells (10⁶/mouse) were injected intravenously in CD45.1-expressing mice.

LSD1 inhibitor DDP 37368 was administered by oral gavage at 45 mg/kg 3 weeks after leukemic cell injection, for 3 consecutive days once a day. Mice were sacrificed 4 days after treatment start and leukemic cells for RNAseq were sorted by flow cytometry after staining for the donor marker CD45.1.

### Results

### LSD1 Inhibitors Induce Marked Upregulation of ERVs and OAS1 Expression.

Inventors have thoroughly investigated the effect of LSD1 inhibitors on models of Acute Promyelocytic Leukemia (APL) (Fioravanti et al., 2020). APL cells share several features of normal Antigen-Presenting Cells (APCs) and can be differentiated into cells with features of dendritic cells (H.-Y. Park et al., 2004). Inventors investigated the transcriptomic changes elicited after 3 days of treatment with LSD1 inhibitor DDP 37368 by oral gavage. Gene Set Enrichment Analysis showed dramatic upregulation of Interferon-related pathways upon LSD1 treatment (FIG. 16A). In particular, genes key to dsRNA sensing (OAS1 and MDA5, FIG. 16B) were among the most upregulated; importantly, inventors did not observe upregulation of IL6 or other inflammatory cytokines. As LSD1 has been recently suggested to modulate interferon responses indirectly through the regulation of ERVs, inventors used a recently developed bioinformatic approach to quantify ERV families in the RNAseq data. Indeed, LSD1 inhibition resulted in significant upregulation of ERVs and other repeated elements (FIG. 16C).

### MHV-Infected Macrophages Secrete Biochemically Distinct Extrinsic Antiviral and Cytotoxic Activities

To investigate cell dynamics following MHV infection, inventors characterized viral titer, cell viability and syncitia formation (a typical but cell type-specific coronavirus-associated cytopathic effect) [Chu H et al, 2020] in L929 fibroblasts and bone marrow-derived macrophages (BMDM) in response to the MHV strain A59 (see Table 1a-c for viral strain confirmation). In both cell types, infection was productive, as demonstrated by a similar viral titer at 24 hours post-infection (hpi) (FIG. 8A). Viability decreased at 48 hpi in both cell types, following comparable MOI-dependent kinetics (FIG. 8B); syncitia were consistently seen in L929 cells, but not in BMDMs (FIG. 8C).

To analyse cellular interactions between L929 fibroblasts and BMDMs, inventors set up a live-cell imaging system that allowed monitoring of cell death and syncitia formation of L929 and BMDM in mono- and co-cocultures. To distinguish L929 cells from BMDMs and to allow precise quantification of syncytia formation, L929 cells were engineered to express H2b-GFP (L929GFP), while cell death was monitored by adding Propidium Iodide (PI). L929^{GFP} and L929WT cells were equally permissive for infection (FIG. 8D). In L929^{GFP} monocultures, it was observed a rapid increase in syncitia numbers (FIG. 1A), starting from ˜12 hpi with a plateau at ˜30 hpi (FIG. 1B), followed by a wave of cell death starting from 33 hpi (FIG. 1C,D). In co-cultures of BMDM and L929^{GFP} cells, L929^{GFP} syncitia formed with kinetics similar to that in monoculture (FIG. 1A,B), while cell death was significantly anticipated by ˜15 hours (FIG. 1C,D), when no MHV-induced cytopathic effect is detectable. In contrast to L929 monoculture, in which death was equally distributed between syncytia and mono-nucleated cells, in cocultures death occurred predominantly outside syncytia (FIG. 8E). Importantly, viral titers were not increased by the presence of BMDM within 24 hpi (FIG. 8A), ruling out an increase in MHV virulence as a cause for early fibroblast death and suggesting the existence of a macrophage-dependent cytotoxic activity.

To characterize secreted activities, supernatants from BMDM infected at different MOI (from 0.001 to 0.1) were collected 24 hpi, UV-inactivated, and tested in progressive 3-fold dilutions on L929 cells, which were infected or not with MHV at MOI 0.1 (see FIG. 1E for a schematic view of the experimental protocol). Supernatants from non-infected BMDMs had no effect on L929 cells, infected or not (FIG. 1F and FIG. 8F). Supernatants from infected BMDMs, in turn, showed complex effects: at high MOI (0.01-0.1) and high concentrations (1:3-1:9 dilution), they strongly reduced syncitia formation (FIG. 1G,I) and viral titer (FIG. 1H), but also induced early cell death, resulting in decreased overall viability (FIG. 1F). Death was MHV-independent, as it was equal in infected and noninfected L929 cells, and started immediately after supernatant addition, a kinetics incompatible with prior viral cytopathic effect (FIG. 1J). At intermediate dilution (1:27), both syncitia formation and early MHV-independent cell death were significantly reduced, resulting in maximal gain in overall viability of infected cells (FIG. 1F). At further dilutions (1:81, 1:243), both antiviral activity (syncitia formation) and MHV-independent death were lost, giving way to late MHV-dependent death (FIG. 1I,J).

These experiments suggested the existence of two biochemically distinct species of secreted activities with antiviral and cytotoxic effect, respectively. To verify this, inventors subjected the supernatants from infected macrophages to size-exclusion chromatography through gel filtration and measured the effects of the supernatant fractions on cell viability of MHV-infected or uninfected cells. The loss of cell viability in uninfected cells was operatively defined as “extrinsic cytotoxic activity” (ECT) and the rescue of cell viability in infected cells as “extrinsic antiviral activity” (EAV). As shown in FIG. 2A-B, the two activities eluted in two clearly separated size ranges, with ECT peaking in fractions 21-27 (corresponding to a predicted protein size of 50-60 kDa) and EAV peaking in fraction 25-31 (corresponding to a predicted protein size of ˜20 kDa). Previous studies identified TNFa and type I interferon as candidates for coronavirus-induced cytotoxic and antiviral activities respectively [Qing H et al, 2020; WHO. Interim Laboratory Biosafety Guidelines for Handling and Processing Specimens Associated with Coronavirus Disease 2019 (COVID-19) 2020. https://www.cdc.gov/coronavirus/2019-ncov/lab/lab-biosafety-guidelines.html]. ELISA showed elevated levels of TNFa and IFNa in the fractions showing maximal ECT and EAV, respectively (FIG. 2C,D), consistent with their expected size (TNF is biologically active in 52 kDa trimers [Lee W S et al, 2020]). Neutralization experiments confirmed the role of TNF and IFNa, since: i) anti-TNFa antibodies dose-dependently rescued viability of both infected and uninfected cells (FIG. 2E); ii) anti-IFNAR antibodies (FIG. 2F) or JAK2 inhibitors (which block IFN signal transmission, FIG. 9A) had no effect on ECT (viability in uninfected cells remained low) and antiviral activity could not be adequately measured due to widespread cell death; iii) dual IFNAR+TNFa blockade resulted in loss of both activities: viability was fully restored in uninfected cells (FIG. 2G), but syncitia became evident in infected cells (FIG. 2H).

These results unequivocally demonstrate that coronavirus-infected macrophages secrete biochemically separated cytotoxic and antiviral activities, identified as TNFa and IFNa respectively. Additionally, supernatant transfer provides an easily tractable model to independently quantitate macrophage extrinsic activities, potentially useful for genetic or pharmaceutical screens

LSD1 Inhibition Abrogates Macrophage Extrinsic Cytotoxic Activity with Minimal Impact on Antiviral Activity

The lysine demethylase LSD1 has been previously implicated in the regulation of NFkB (the main driver of TNFa expression) and type I Interferon in LPS-induced inflammation and cancer respectively [Kim D et at, 2018; Sheng W et al, 2018], so inventors tested its direct involvement in coronavirus response. To this end, the effect of the LSD1 inhibitor DDP38003 was tested [Vianello P et al, 2016] (henceforth “DDP”) on BMDM-secreted extrinsic antiviral and cytotoxic activities. Importantly, DDP had no or negligible effect on cell viability per se at the doses employed (supp FIG. 3A).

Supernatants from MHV-infected BMDMs, treated with vehicle (snDMSO) or DDP (snDDP) at medium (2.5 μM) or high (10 μM) concentrations, were added to uninfected or MHV-infected L929 cells (MOI 0.1). Strikingly, snDDP rescued completely the extrinsic cytotoxic effect of the BMDM supernatants on both uninfected and infected L929 cells, as shown by restored viability (FIG. 3A,B) and abrogation of early cell death (FIG. 3D,E), similar to what obtained with maximal doses of anti TNFa (FIG. 2E). However, supernatant activity was not completely reversed and antiviral activity was not significantly compromised, since viral titer was still reduced by ˜2 logs upon addition of snDDP (FIG. 3C) and syncitia formation remained visibly less pronounced than in the absence of supernatant (FIG. 3F). Size-exclusion chromatography confirmed that DDP 2.5 μM completely eliminated the cytotoxic activity eluting at fractions 23-25 while maintaining antiviral activity at fractions 27-31 (FIG. 3G,J). TNFa became undetectable upon treatment of BMDM with DDP at 2.5 μM (FIGS. 3H,K), whereas IFN was preserved at 2.5 μM and only significantly reduced at 10 μM (FIG. 3I,L). Anti-TNFa antibodies had no effect on snDDP, consistently with complete DDP-dependent abrogation of TNFa secretion (FIG. 10B) whereas both anti-IFNAR antibodies or pharmacological JAK inhibition abrogated antiviral activity (FIG. 10C,E) and syncitia developed similarly to untreated cells (FIG. 3M).

To genetically confirm the role of LSD1, we silenced LSD1 by RNA interference in the RAW264.7 macrophage cell line. Similarly to BMDMs, RAW264.7 cells could be productively infected by MHV-A59 (FIG. 10G); EAV was negligible (FIG. 10H) but ECT was significant (80% viability loss in uninfected L929 cells, FIG. 10H). Consistent with DDP data, shLSD1 abrogated ECT and completely rescued viability of L929 cells exposed to infected RAW supernatant (FIG. 10H).

Finally, to provide a minimal representation of the cellular landscape of in vivo coronavirus airborne infections, inventors also tested the impact of snBMDM on the lung epithelial cell line LA4, previously shown to be susceptible to MHV-A59 infection [VanLeuven J T et al, 2017]. LA4 were not sensitive to BMDM ECT but did indeed show loss of viability upon MHV infection; this was partially reversed by snDDP (40% viability with snDDP 2.5 uM vs 20% in snDMSO) (FIG. 10I).

The above experiments demonstrate that LSD1 is required for the secretion of the TNFa-associated ECT but has little impact on the Interferon-dependent EAV.

### LSD1 Inhibition in Macrophages Abrogates MHV-Induced NFKB Transcriptional Program but Maintains the Interferon Program.

To investigate molecular mechanisms underlying the macrophage response to MHV and the role of LSD1, inventors performed RNAseq analyses of macrophages treated with DMSO or DDP at moderate (2.5 μM) or high (10 μM) concentrations and infected or not with MHV-A59 MOI 0.1, at 24 hpi.

Hierarchical clustering clearly separated infection and treatment groups, and revealed: i) a strong transcriptional effect of the viral infection; ii) a relatively modest impact of DDP on basal transcription; and iii) a strong and dose-dependent effect of DDP on MHV-dependent transcription (FIG. 4A and FIG. 11A). Seven transcriptional clusters were clearly demarcated, which were assigned to two groups, containing, respectively, genes up- (groups A1-4) or down- (groups B1-3) regulated by the infection. DDP largely antagonized the transcriptional effect of MHV-infection. In particular, in 5 of the 7 clusters, DDP markedly attenuated the down- (B2) or up- (A1,2) regulations induced by the infection, or even inverted their regulation (A3, B1). In only two clusters (A4, B3), instead, DDP intensified the up- (A4) or down- (B3) regulations induced by the infection (FIG. 3A,C). Gene ontology and motif-finding analyses revealed significant enrichment of distinct biological functions and transcription factors in each of the different clusters (FIG. 3B). As downregulation of the extrinsic cytotoxic activity is the dominant effect of DDP treatment, inventors initially focused on clusters containing genes whose MHV-dependent upregulation is inhibited by DDP (clusters A1-3). These clusters were enriched for proinflammatory cytokines and NFKB binding motifs (cluster A1), Interferon-stimulated genes and IRF binding motifs (cluster A2) and genes involved in granule formation and Sp 1 binding motifs (cluster A3) (FIG. 3B). Cluster A1 included all cytokines currently implicated in the severe form of Covid19 (IL1a, Il1b, IL6, TNFa;) and exhibited the strongest quantitative changes: it was the most highly upregulated in response to MHV (average of ˜8 fold) and the most downregulated in response to DDP (reaching baseline levels at 10 μM). The impact of DDP on IRF-associated genes in cluster A2 was significantly less pronounced: the extent of the MHV-induced upregulations was comparable to A1, but even with DDP 10 μM A3 cluster genes remained on average upregulated ˜5 fold (FIG. 3C). In further support of a direct activity of DDP on NFKB, we observed extensive overlaps between genes of the A1-A4 clusters and the reported LPS-induced and LSD1-regulated NFKB-targets [Kim D et al, 2018] (FIG. 11B). Gene expression analyses by qRT-PCR of representative NFKB-dependent cytokines (Il1b, TNFa, Il6) showed rapid induction at 8-12 hpi and strong down-regulation by DDP (FIG. 3D). In turn, the impact of DDP on the expression of IFNa and representative ISGs (Isg15 and Ifit1) was significantly less pronounced (FIG. 3E). Notably, IFNa transcription was detected after that of Isg15 and Ifit1, suggesting an Interferon-independent initiation of ISG expression, a phenomenon previously reported in West Nile virus infections [Siren J et al 2005].

These results further support the finding that LSD1 acts as a fundamental cofactor of the NFKB-dependent transcriptional response but has minimal effect on the interferon response.

### LSD1 Inhibition Inhibits NFKB Nuclear Translocation and Target Binding but Spares IRF1

Activation of both NFKB and IRFs is associated with nuclear localization [Hayden M S et al, 206; Honda K et al, 2006]. Thus, levels of nuclear NFKB and IRFs by biochemical fractionation and immunofluorescence were investigated.

Western blotting analyses of nuclear/cytoplasmic fractions showed rapid nuclear re-localization of NFKB after MHV-infection, as expected, already evident at 10 hpi (FIG. 5A-B). Same analysis for all 9 IRFs (FIG. 12A) showed upregulation and nuclear localization of IRF1 and, to a lesser extent, IRF2, whereas the other IRFs showed either no change (IRF5,9) or even decreased nuclear levels (IRF 3,4,6,7,8). Notable is the suppression of IRF3 and IRF7, typically involved in the response to dsRNA and ssRNA, thus confirming the existence of cellular-evasive mechanisms specific to coronaviruses [Zhou H et al, 2007; Karnam G et al, 2012; Kindler E et al, 2017]. Results obtained by biochemical fractionation were confirmed by immunofluorescence analyses using anti-NFKB and -IRF1 (FIG. 5C,D) antibodies, which showed nuclear localization of both factors upon MHV-infection. Notably, differential analyses of infected or uninfected cells (the former identified by NSP9 positivity) showed nuclear localization of both NFKB and IRF1 also in NSP9-negative cells, suggesting activation of paracrine loops (FIG. 5C-D). Treatment of MHV-infected cells with DDP abrogated nuclear NFKB but had a much less pronounced effect on IRF1, resulting in barely any difference at 24 hpi in fractionation experiments (FIG. 5B) and a nuclear signal persistently above the baseline by immunofluorescence, both qualitatively and quantitatively (FIG. 5C,D).

Chromatin Immunoprecipitation (ChIP) showed that MHV infection induced binding of both NFKB and LSD1 at the promoter of NFKB target genes, which was abrogated by DDP (FIG. 5E).

Collectively, these results demonstrate that LSD1 is specifically implicated in regulating MHV-induced NFKB nuclear relocalization, leaving relatively unaltered the IRF1 response.

LSD1 Inhibition Exerts Intrinsic Antiviral Activity Associated with Activation of a Subset of ISGs and Lysosome Acidification

In addition to the secreted (extrinsic) antiviral activity induced by DDP in MHV-infected BMDMs, we explored whether LSD1 inhibition also exerts a direct (intrinsic) antiviral activity, by measuring survival, titer and syncitia formation in L929, BMDMs and LA4 cells.

In all three cell types, treatment with DDP increased cell survival in a dose-dependent manner (FIG. 6A-B and FIG. 13) and reduced viral titer and syncitia formation (FIG. 6C-D and FIG. 13A, B, D, F). The magnitude of the antiviral activity was cell type-specific and most evident in L929 cells, where the titer was reduced by ˜1 log at 2.5 μM (FIG. 6C-D). In LA4 and BMDM, instead, a relevant effect was only evident at the higher dose of 10 μM (FIG. 13D,F). Importantly, LSD1 ablation by either CRISPR-Cas9 (FIG. 7E-F and FIG. 14A) or RNA interference (FIG. 14B) abrogated MHV-induced cytotoxicity and significantly reduced viral titer by at least 2 logs in L929 cells.

The mechanisms of the direct anti-viral effect of Lsd1 inhibition was then investigated. L929 cells do not produce any type I interferon in response to MHV, irrespective of DDP treatment (FIG. 14C). Consistently, JAK inhibition did not antagonize the DDP protective effect (FIG. 14B, D). Of note, a subset of ISGs (Ifit1, Oasl1, Mx2 among those tested) was modestly activated upon MHV and further upregulated by DDP in a dose-dependent manner (FIG. 6G), suggesting that ISG activation in DDP-treated L929 cells occurs in an Interferon-independent manner.

To further characterize the intrinsic antiviral activity of DDP inventors took advantage of BMDM RNAseq data, and focused on pathways enriched in clusters of the B group, which include genes that are either down-regulated or modestly affected by MHV, yet strongly upregulated by DDP treatment. These clusters were enriched for gene sets involved in vesicle biogenesis and lysosome biology (FIG. 3A-B). Coronavirus egress has been recently shown to occur through an unconventional lysosome-dependent pathway, which requires inhibition of lysosomal acidification and is associated with release of the ER marker BiP [Ghosh S et al, 2020]. In agreement, inventors observed downregulation by MHV and upregulation by DDP of the lysosomal ATP6v1a (FIG. 14E), a component of the vacuolar ATPase that mediates acidification of the organelle. In both BMDMs and L929 cells, DDP led to significant acidification of the lysosomal compartment, as measured by the pH-sensitive dye lysosensor, completely overriding the modest yet significant decrease in lysosomal acidification associated with MHV infection in L929 cells (FIG. 6H and FIG. 14F). Extracellular release of the endoplasmic reticulum chaperone BiP was reduced upon DDP treatment in both macrophages and L929 cells (FIG. 6I and FIG. 14G).

Together, these data suggest that LSD1 ablation allows the emergence of a cell-intrinsic antiviral response characterized by interferon-independent ISG activation and restoration of lysosomal acidification, resulting in reduced viral release. The precise point of regulation of this activity remains to be elucidated.

### LSD1 is a Valid Therapeutic Target to Prevent Human Cytokine Storm in COVID-19

To explore the translational relevance of LSD1 inhibition as a potential treatment for Covid19 or other coronavirus infections, inventors compared the activity of DDP with ORY-1001 and dexamethasone. ORY-1001 is an LSD1 inhibitor of the same class of DDP that has completed initial phases of clinical development in the context of hematological malignances [Fu D-J et al, 2020]. Dexamethasone is the only medical treatment approved to date for Covid-19 as it moderately improves survival and effectively dampens the NFKB-dependent response, but is also suspected to suppress interferon activity thus resulting in prolonged viral shedding [Flammer J R et al, 2010; Jalkanen J et al, 2020; Cano E J et al, 2020].

All three compounds inhibited the macrophage-secreted cytotoxic activity in a dose-dependent manner, as measured by restoration of cell viability of non-infected L929 cells exposed to BMDM-conditioned supernatants (FIG. 7A). When the same supernatants were added to infected L929 cells, even at the highest concentrations, neither of the two LSD1 inhibitors decreased the macrophage-secreted antiviral activity, as judged by restoration of cell viability and lack of syncitia formation. Dexamethasone, instead, at cytotoxic-suppressing dosages failed to recover viability and to prevent syncitia (10 μM, FIG. 7B,D). Similar results were obtained when the three drugs were applied directly on infected L929 cells: both DDP and Ory1001, but not dexamethasone, elicited intrinsic antiviral activity (FIG. 7C-D).

Then, it was the tested pharmacological interactions with type I Interferon, relevant for mechanistic understanding and potential pharmacological combinations. IFNa alone dose-dependently inhibited MI-IV activity and viral titer, and no synergism nor antagonism could be identified if cells were co-treated with increasing doses of DDP (FIG. 15A,B). Thus, although high-dose DDP does indeed reduce Interferon production by macrophages (FIG. 3J), it does not interfere with downstream interferon-dependent signaling.

Similar experiments were translated in the human setting. As mentioned above, the possibility to directly study the response of human macrophages in vitro to SARS-CoV2 remains challenging. As reported in literature [Dalskov L et al 2020], attempts to infect monocyte-derived macrophages in vitro have been unsuccessful (FIG. 15C). Transcriptomic data from broncho-alveolar lavage of Covid19 patients showed that in severe Covid19 patients, alveolar macrophages host actively replicating SARS-CoV2 (as shown by the detection of negative strand RNA reads), and that the transcriptional profile of infected Tissue-Resident Alveolar Macrophages (TRAM2) differ significantly from uninfected TRAMs (TRAM1) within the same patients. Inventors further characterized differentially expressed genes between TRAM1 and TRAM2 and found a striking overlap between TRAM2-overexpressed DEGs and genes of our clusters 1 (NFKB-enriched) and 6 (IRF-enriched). Mouse homologs of human TRAM2 were, expectedly, similarly impacted by DDP treatment in the mouse transcriptomic data, with cluster 1 homologs (NFKB-associated) significantly more downregulated than cluster 6 homologs (IRF1-associated). Inventors compared the effect of DDP and dexamethasone on human monocyte-derived macrophages stimulated with agents inducing antiviral innate responses to dsRNA (polyIC) and ssRNA (R848). Again, whereas Dexamethasone led to generalized reduction of proinflammatory cytokines TNFa and IL6, DDP maintained selectivity with a significantly stronger reduction in the transcription of NFKB-dependent cytokines than ISGs (FIG. 7F), demonstrating a general, trans-species and stimulus-independent selective effect of DDP on the inflammatory and interferon cascade.

### Discussion

The invention provides a systematic characterization of the transcriptional responses to the MHV-A59 coronavirus in mouse macrophages and demonstrates that this model system is relevant for investigating human SARS-CoV2, as the transcriptional responses elicited by MHV-infected murine macrophages are highly similar to those activated in infected alveolar macrophages in severe Covid-19 patients. They include strong and early activation of NFKB-dependent production of proinflammatory cytokines, and less intense and slower activation of type I interferon and ISGs. Inventors identified IRF1 as the key activator of the interferon response and reveal a crucial role for the lysine demethylase LSD1 in dampening the NFKB-dependent arm, providing a mechanistic rationale to test LSD1 inhibitors, a class of drugs developed for oncological indications, for the treatment of Coronavirus infections.

The finding that IRF1 and to a lesser extent IRF2 are the only IRFs that clearly change their intracellular localization in response to MHV infection is perhaps surprising, given the widespread involvement of other IRF members in the antiviral response to many viruses. However, although generally considered dispensable for the activation of antiviral responses [Honda K et al, 2006], IRF1 can lead to early activation of ISGs and type I Interferon, independently from other IRFs that typically occur as a later event, such as dsRNA-MDA5/RIG-I activated IRF3 [Pulit-Penaloza J A et al, 2012; Schoggins J W et al, 2011; Panda D et al, 2019]. In the absence of other viral-induced effects, this would lead to Interferon-loop amplification, inhibition of TNF signaling or IL1b production and attenuation of NFKB signaling [Guarda G et al, 2011; Park S H et al, 2017]. However, in the presence of additional viral-induced mechanisms of evasion of the Interferon response, the late IRF3-dependent amplification of Interferon production and the cross-inhibition of the NFKB loop is inhibited, thus favoring inflammation over Interferon-induced viral clearance.

The invention clearly demonstrates that LSD1 plays a key role in the amplification of the NFKB response to MHV, allowing elevated and sustained production of IL1b, TNFa, IL6 and other proinflammatory cytokines. The results obtained expand a prior study showing that LSD1 is phosphorylated in response to LPS and induces NFKB demethylation and its persistence in the nucleus (Kim et al, 2018). Likewise, MHV, with MHV infection induced recruitment of LSD1 to NFKB target-genes, as shown by ChIP and immunofluorescence. Further research is required to elucidate if histone demethylase activity is required for the effect of LSD1 in the NFKB response, and if main targets are NFKB itself or critical effector proteins encoded by NFKB target-genes.

An intriguing finding is the existence of an LSD1-dependent, interferon-independent cell-intrinsic response that results in the activation of a subset of interferon-stimulated genes and inhibition of lysosomal-mediated virus egress. The precise nature of this response, which may contribute to the initial, Interferon-independent trigger of IRF1, requires further investigation and may involve direct histone demethylation at regulatory elements of ISGs and/or genes involved in lysosomal activity.

Perhaps unexpectedly, inventors did not find significant upregulation of dsRNA-forming ERVs upon LSD1 inhibition in uninfected cells, suggesting that mechanisms responsible for ERV suppression in non-transformed myeloid cells are significantly different from those operating in transformed cells, where LSD1 ablation leads instead to strong ERV upregulation [Sheng W et al, 2018]. On the contrary, LSD1 inhibition actively suppressed ERV upregulation secondary to MHV infection. Notably, it has been recently reported that inflammation following SARS-CoV2 infection leads to ERV upregulation in human cells [Zhang L et al, 2020], reinforcing the conclusion that suppression of the MHV-induced inflammatory response is the dominant effect of LSD1 inhibition.

NFKB and IRF factors are known to intersect at multiple levels, most notably in the transcriptional regulation of type I interferon genes in the so-called “enhanceosome”, which critically relies on the simultaneous binding of NFKB and IRF1 or IRF9 to IFN enhancer regulatory-elements [Schafer S L et al, 1998; Thanos D et al, 1995]. The need for cooperation between these two factors may provide a mechanistic explanation for the differential activity of steroids vs LSD1 inhibitors: whereas steroids inhibit NFKB by preventing NFKB nuclear translocation through upregulation of IkB [Auphan N et al, 1995], LSD1 has been proposed to suppress the degradation of nuclear-translocated NFKB [Kim D et al, 2018]. Thus, LSD1 inhibition may allow minute amounts of NFKB to enter the nucleus for a restricted time window, allowing it to act as a “pioneer” transcription factor to render chromatin of the IRF1 target loci accessible for subsequent binding of canonical transcriptional activators.

As a matter of fact, the present invention provides multiple LSD1 inhibitors exhibiting strong anti-inflammatory activity in virus-infected macrophages, both mouse and human, without however suppressing their antiviral responses, as instead occurs with dexamethasone, the only drug approved for Covid to date. Thus, LSD1 inhibition is a highly promising strategy to prevent Covid19 progression. In particular, LSD1 inhibits NFKB-dependent gene expression from a variety of signals that mimic viral RNA recognition, yet it showed moderate effects on Interferon secretion and response, suggesting a generalized positive effects on coronavirus infections. This strategy is further supported by findings in murine models of SARS-CoV respiratory diseases, in which pharmacological inhibition of NFKB or generation of virus mutants inducing attenuated NFKB activation led to decreased disease severity and prolonged survival [DeDiego M L et al, 2014]. In addition to LSD1 inhibition in monotherapy, the results showed in the present invention provide a clear basis for the synergism of LSD1 inhibitors with Interferon.

The results obtained also confirmed that in vitro-differentiated human macrophages are not productively infected nor significantly activated when exposed to SARS-CoV2, despite recent findings that alveolar macrophages in Covid19 patients host elevated amounts of actively replicating virus. While further research is required to identify the factors that determine in vivo susceptibility of human macrophage to coronavirus infection, the mouse system characterized in the present study represents a useful model to further investigate conserved host innate responses to coronaviruses.

### REFERENCES

Auphan N, DiDonato J A, Rosette C, Helmberg A, Karin M. Immunosuppression by glucocorticoids: inhibition of NF-kappa B activity through induction of I kappa B synthesis. Science 1995; 270:286-90. https://doi.org/10.1126/science.270.5234.286
Bagnall J, Boddington C, England H, Brignall R, Downton P, Alsoufi Z, et al. Quantitative analysis of competitive cytokine signaling predicts tissue thresholds for the propagation of macrophage activation. Sci Signal 2018; 11:eaaf3998. https://doi.org/10.1126/scisignal.aaf3998
Banerjee, S., Gusho, E., Gaughan, C., Dong, B., Gu, X., Holvey-Bates, E., Talukdar, M., Li, Y., Weiss, S. R., Sicheri, F., Saunthararajah, Y., Stark, G. R., & Silverman, R. H. (2019). OAS-RNase L innate immune pathway mediates the cytotoxicity of a DNA-demethylating drug. Proceedings of the National Academy of Sciences of the United States of America, 116(11), 5071-5076. https://doi.org/10.1073/pnas.1815071116
Bastard P, Rosen L B, Zhang Q, Michailidis E, Hoffmann H-H, Zhang Y, et al. Autoantibodies against type I IFNs in patients with life-threatening COVID-19. Science (80) 2020; 370:eabd4585. https://doi.org/10.1126/science.abd4585
Birdwell, L. D., Zalinger, Z. B., Li, Y., Wright, P. W., Elliott, R., Rose, K. M., Silverman, R. H., & Weiss, S. R. (2016). Activation of RNase L by Murine Coronavirus in Myeloid Cells Is Dependent on Basal Oas Gene Expression and Independent of Virus-Induced Interferon. Journal of Virology, 90(6), 3160-3172. https://doi. org/10.1128/jvi.03036-15
Brocks, D., Schmidt, C. R., Daskalakis, M., Jang, H. S., Shah, N. M., Li, D., Li, J., Zhang, B., Hou, Y., Laudato, S., Lipka, D. B., Schott, J., Bierhoff, H., Assenov, Y., Helf, M., Ressnerova, A., Islam, M. S., Lindroth, A. M., Haas, S., . . . Plass, C. (2017). DNMT and
Cano E J, Fuentes X F, Campioli C C, O'Horo J C, Saleh O A, Odeyemi Y, et al. Impact of Corticosteroids in Coronavirus Disease 2019 Outcomes: Systematic Review and Meta-analysis. Chest 2020. https://doi.org/10.1016/j.chest.2020.10.054
Canton J, Fehr A R, Fernandez-Delgado R, Gutierrez-Alvarez F J, Sanchez-Aparicio M T, Garcia-Sastre A, et al. MERS-CoV 4b protein interferes with the NF-KB-dependent innate immune response during infection. PLOS Pathog 2018; 14:e1006838
Cao, B., Wang, Y., Wen, D., Liu, W., Wang, J., Fan, G., Ruan, L., Song, B., Cai, Y., Wei, M., Li, X., Xia, J., Chen, N., Xiang, J., Yu, T., Bai, T., Xie, X., Zhang, L., Li, C., . . . Wang, C. (2020). A Trial of Lopinavir-Ritonavir in Adults Hospitalized with Severe Covid-19. The New England Journal of Medicine. https://doi.org/10.1056/NEJMoa2001282
Cella, M., Salio, M., Sakakibara, Y., Langen, H., Julkunen, I., & Lanzavecchia, A. (1999). Maturation, activation, and protection of dendritic cells induced by double-stranded RNA. The Journal of Experimental Medicine, 189(5), 821-829. https://doi.org/10.1084/jem.189.5.821
Chen, C., Pan D., Deng A M., Huang F., Sun B., Yang R. (2013). DNA methyltransferases 1 and 3B are required for hepatitis C virus infection in cell culture. Virology, 441, 57-65. http://dx.doi.org/10.1016/j.virol.2013.03.005
Chu H, Chan J F-W, Yuen T T-T, Shuai H, Yuan S, Wang Y, et al. Comparative tropism, replication kinetics, and cell damage profiling of SARS-CoV-2 and SARS-CoV with implications for clinical manifestations, transmissibility, and laboratory studies of COVID-19: an observational study. The Lancet Microbe 2020; 1:e14-23. https://doi.org/10.1016/S2666-5247(20)30004-5
Chiappinelli, K. B., Strissel, P. L., Desrichard, A., Li, H., Henke, C., Almon, B., Hein, A., Rote, N. S., Cope, L. M., Snyder, A., Makarov, V., Buhu, S., Slamon, D. J., Wolchok, J. D., Pardoll, D. M., Beckmann, M. W., Zahnow, C. A., Mergoub, T., Chan, T. A., . . . Strick, R. (2015). Inhibiting DNA Methylation Causes an Interferon Response in Cancer via dsRNA Including Endogenous Retroviruses. Cell, 162(5), 974-986. https://doi.org/10.1016/j.cell.2015.07.011
Chu, H., Zhou, J., Ho-Yin Wong, B., Li, C., Cheng, Z. S., Lin, X., Kwok-Man Poon, V., Sun, T., Choi-Yi Lau, C., Fuk-Woo Chan, J., Kai-Wang To, K., Chan, K. H., Lu, L., Zheng, B. J., & Yuen, K. Y. (2014). Productive replication of Middle East respiratory syndrome coronavirus in monocyte-derived dendritic cells modulates innate immune response. Virology, 454-455(1), 197-205. https://doi.org/10.1016/j.virol.2014.02.018
Cui, J., Li, F., & Shi, Z.-L. (2019). Origin and evolution of pathogenic coronaviruses. Nature Reviews. Microbiology, 17(3), 181-192. https://doi.org/10.1038/s41579-018-0118-9
Dalskov L, Møhlenberg M, Thyrsted J, Blay-Cadanet J, Poulsen E T, Folkersen B H, et al. SARS-CoV-2 evades immune detection in alveolar macrophages. EMBO Rep 2020; 21:e51252. https://doi.org/https://doi.org/10.15252/embr.202051252
Daskalakis, M., Brocks, D., Sheng, Y. H., Islam, M. S., Ressnerova, A., Assenov, Y., Milde, T., Oehme, I., Witt, O., Goyal, A., Kuhn, A., Hartmann, M., Weichenhan, D., Jung, M., & Plass, C. (2018). Reactivation of endogenous retroviral elements via treatment with DNMT- and HDAC-inhibitors. Cell Cycle, 17(7), 811-822. https://doi.org/10.1080/15384101.2018.1442623
DeDiego M L, Nieto-Torres J L, Regla-Nava J A, Jimenez-Guarderio J M, Fernandez-Delgado R, Fett C, et al. Inhibition of NF-KB-mediated inflammation in severe acute respiratory syndrome coronavirus-infected mice increases survival. J Virol 2014; 88:913-24. https://doi.org/10.1128/JVI.02576-13.
Del Valle D M, Kim-Schulze S, Huang H-H, Beckmann N D, Nirenberg S, Wang B, et al. An inflammatory cytokine signature predicts COVID-19 severity and survival. Nat Med 202026:1636-43. https://doi.org/10.1038/s41591-020-1051-9
Deng, X., Hackbart, M., Mettelman, R. C., O'Brien, A., Mielech, A. M., Yi, G., Kao, C. C., & Baker, S. C. (2017). Coronavirus nonstructural protein 15 mediates evasion of dsRNA sensors and limits apoptosis in macrophages. Proceedings of the National Academy of Sciences of the United States of America, 114(21), E4251-E4260. https://doi.org/10.1073/pnas.1618310114
Diamantopoulos P. T., Michael M., Benopoulou O., Bazanis F., Tzeletas G., Meletis J., Vayopoulos G., and Nora-Athina Viniou (2012). Antiretroviral activity of 5-azacytidine during treatment of a HTLV-1 positive myelodysplastic syndrome with autoimmune manifestations. Virology Journal, 9:1. doi: 10.1186/1743-422X-9-1.
Fenaux, P., Mufti, G. J., Hellstrom-Lindberg, E., Santini, V., Finelli, C., Giagounidis, A., Schoch, R., Gattermann, N., Sanz, G., List, A., Gore, S. D., Seymour, J. F., Bennett, J. M., Byrd, J., Backstrom, J., Zimmerman, L., McKenzie, D., Beach, C. L., & Silverman, L. R. (2009). Efficacy of azacitidine compared with that of conventional care regimens in the treatment of higher-risk myelodysplastic syndromes: a randomised, open-label, phase III study. The Lancet Oncology, 10(3), 223-232. https://doi.org/https://doi.org/10.1016/S1470-2045(09)70003-8
Fioravanti, R., Romanelli, A., Mautone, N., Di Bello, E., Rovere, A., Corinti, D., Zwergel, C., Valente, S., Rotili, D., Botrugno, O. A., Dessanti, P., Vultaggio, S., Vianello, P., Cappa, A., Binda, C., Mattevi, A., Minucci, S., Mercurio, C., Varasi, M., & Mai, A. (2020). Tranylcypromine-Based LSD1 Inhibitors: Structure-Activity Relationships, Antiproliferative Effects in Leukemia, and Gene Target Modulation. Chem Med Chem. https://doi.org/10.1002/cmdc.201900730
Flammer J R, Dobrovolna J, Kennedy M A, Chinenov Y, Glass C K, Ivashkiv L B, et al. The Type I Interferon Signaling Pathway Is a Target for Glucocorticoid Inhibition. Mol Cell Biol 2010; 30:4564 LP-4574. https://doi.org/10.1128/MCB.00146-10
Fung T S, Liu D X. Human coronavirus: Host-pathogen interaction. Annu Rev Microbiol 2019; 73:529-60. https://doi.org/10.1146/annurev-micro-020518-115759
Gantier, M. P., & Williams, B. R. G. (2007). The response of mammalian cells to double-stranded RNA. Cytokine & Growth Factor Reviews, 18(5-6), 363-371. https://doi.org/10.1016/j.cytogfr.2007.06.016
Ghosh S, Dellibovi-Ragheb T A, Kerviel A, Pak E, Qiu Q, Fisher M, et al. &#x3b2;-Coronaviruses Use Lysosomes for Egress Instead of the Biosynthetic Secretory Pathway. Cell 2020. https://doi.org/10.1016/j.cell.2020.10.039
Guarda G, Braun M, Staehli F, Tardivel A, Mattmann C, Förster I, et al. Type I Interferon Inhibits Interleukin-1 Production and Inflammasome Activation. Immunity 2011; 34:213-23. https://doi.org/10.1016/j.immuni.2011.02.006
Gurevich, A., Saveliev, V., Vyahhi, N., and Tesler, G. (2013). QUAST: Quality assessment tool for genome assemblies. Bioinformatics 29, 1072-1075.
Hall J C, Rosen A. Type I interferons: crucial participants in disease amplification in autoimmunity. Nat Rev Rheumatol 2010; 6:40-9. https://doi.org/10.1038/nrrheum.2009.237.
Haw, D. J., Cummings, D. A. T., Lessler, J., Salje, H., Read, J. M., & Riley, S. (2019). Differential mobility and local variation in infection attack rate. PLoS Computational Biology, 15(1), e1006600. https://doi.org/10.1371/journal.pcbi.1006600
Hadjadj J, Yatim N, Barnabei L, Corneau A, Boussier J. Impaired type I interferon activity and inflammatory responses in severe COVID-19 patients 2020; 724:718-24
Hayden M S, West A P, Ghosh S. NF-κB and the immune response. Oncogene 2006; 25:6758-80. https://doi.org/10.1038/sj.onc.1209943
Hirai A, Ohtsuka N, Ikeda T, Taniguchi R, Blau D, Nakagaki K, et al. Role of Mouse Hepatitis Virus (MHV) Receptor Murine CEACAM1 in the Resistance of Mice to MHV Infection: Studies of Mice with Chimeric mCEACAM1a and mCEACAM1b. J Virol 2010; 84:6654 LP-6666. https://doi.org/10.1128/JVI.02680-09
Honda K, Takaoka A, Taniguchi T. Type I Interferon Gene Induction by the Interferon Regulatory Factor Family of Transcription Factors. Immunity 2006; 25:349-60. https://doi.org/10.1016/j.immuni.2006.08.009
Jalkanen J, Pettilä V, Huttunen T, Hollmén M, Jalkanen S. Glucocorticoids inhibit type I IFN beta signaling and the upregulation of CD73 in human lung. Intensive Care Med 2020; 46:1937-40. https://doi.org/10.1007/s00134-020-06086-3
Johnson, N. R., Yeoh, J. M., Coruh, C., & Axtell, M. J. (2016). Improved Placement of Multimapping Small RNAs. G3 (Bethesda, Md.), 6(7), 2103-2111. https://doi.org/10.1534/g3.116.030452
Karnam G, Rygiel T P, Raaben M, Grinwis G C M, Coenjaerts F E, Ressing M E, et al. CD200 receptor controls sex-specific TLR7 responses to viral infection. PLoS Pathog 2012; 8:e1002710. https://doi.org/10.1371/journal.ppat.1002710
Kassiotis, G., & Stoye, J. P. (2016). Immune responses to endogenous retroelements: taking the bad with the good. Nature Reviews. Immunology, 16(4), 207-219. https://doi.org/10.1038/nri.2016.27
Kim D, Nam H J, Lee W, Yim H Y, Ahn J Y, Park S W, et al. PKCα-LSD1-NF-κB-Signaling Cascade Is Crucial for Epigenetic Control of the Inflammatory Response. Mol Cell 2018; 69:398-411.e6. https://doi.org/10.1016/j.molce1.2018.01.002
Kindler, E., Gil-Cruz, C., Spanier, J., Li, Y., Wilhelm, J., Rabouw, H. H., Züst, R., Hwang, M., V'kovski, P., Stalder, H., Marti, S., Habjan, M., Cervantes-Barragan, L., Elliot, R., Karl, N., Gaughan, C., van Kuppeveld, F. J. M., Silverman, R. H., Keller, M., . . . Thiel, V. (2017). Early endonuclease-mediated evasion of RNA sensing ensures efficient coronavirus replication. PLoS Pathogens, 13(2), 1-26. https://doi.org/10.1371/journal.ppat.1006195
Knoops, K., Kikkert, M., Van Den Worm, S. H. E., Zevenhoven-Dobbe, J. C., Van Der Meer, Y., Koster, A. J., Mommaas, A. M., & Snijder, E. J. (2008). SARS-coronavirus replication is supported by a reticulovesicular network of modified endoplasmic reticulum. PLoS Biology, 6(9), 1957-1974. https://doi.org/10.1371/journal.pbio.0060226
Kopecky-Bromberg, S. A., Martinez-Sobrido, L., Frieman, M., Baric, R. A., & Palese, P. (2007). Severe acute respiratory syndrome coronavirus open reading frame (ORF) 3b, ORF 6, and nucleocapsid proteins function as interferon antagonists. Journal of Virology, 81(2), 548-557. https://doi.org/10.1128/JVI.01782-06
Lawrence T, Natoli G. Transcriptional regulation of macrophage polarization: enabling diversity with identity. Nat Rev Immunol 2011; 11:750-61. https://doi.org/10.1038/nri3088Lech M, Avila-Ferrufino A, Skuginna V, Susanti H E, Anders H-J. Quantitative expression of RIG-like helicase, NOD-like receptor and inflammasome-related mRNAs in humans and mice. Int Immunol 2010; 22:717-28. https://doi.org/10.1093/intimm/dxq058
Lee W S, Wheatley A K, Kent S J, DeKosky B J. Antibody-dependent enhancement and SARS-CoV-2 vaccines and therapies. Nat Microbiol 2020; 5:1185-91. https://doi.org/10.1038/s41564-020-00789-5
Liang Y., Quenelle D., Vogel J. L., Mascaro C., Ortega A, Kristie T. M. (2013). A Novel Selective LSD1/KDM1A Inhibitor Epigenetically Blocks Herpes Simplex Virus Lytic Replication and Reactivation from Latency. mBio, 4(1):e00558-12. doi: 10.1128/mBio.00558-12.
Macfarlan, T. S., Gifford, W. D., Agarwal, S., Driscoll, S., Lettieri, K., Wang, J., Andrews, S. E., Franco, L., Rosenfeld, M. G., Ren, B., & Pfaff, S. L. (2011). Endogenous retroviruses and neighboring genes are coordinately repressed by LSD1/KDM1A. Genes & Development, 25(6), 594-607. https://doi.org/10.1101/gad.2008511
Merad M, Martin J C. Pathological inflammation in patients with COVID-19: a key role for monocytes and macrophages. Nat Rev Immunol 2020; 20:355-62. https://doi.org/10.1038/s41577-020-0331-4
Narayanan, K., Huang, C., Lokugamage, K., Kamitani, W., Ikegami, T., Tseng, C.-T. K., & Makino, S. (2008). Severe acute respiratory syndrome coronavirus nsp1 suppresses host gene expression, including that of type I interferon, in infected cells. Journal of Virology, 82(9), 4471-4479. https://doi.org/10.1128/JVI.02472-07
Nehme, Z., Pasquereau, S., & Herbein, G. (2019). Control of viral infections by epigenetic-targeted therapy. Clinical Epigenetics, 11(1), 55. https://doi.org/10.1186/s13148-019-0654-9
Nurk, S., Bankevich, A., Antipov, D., Gurevich, A., Korobeynikov, A., Lapidus, A., Prjibelsky, A., Pyshkin, A., Sirotkin, A., Sirotkin, Y., et al. (2013). Assembling Genomes and Mini-metagenomes from Highly Chimeric Reads. pp. 158-170.
Panda D, Gjinaj E, Bachu M, Squire E, Novatt H, Ozato K, et al. IRF1 Maintains Optimal Constitutive Expression of Antiviral Genes and Regulates the Early Antiviral Response. Front Immunol 2019; 10:1019. https://doi.org/10.3389/fimmu.2019.01019
Park, H.-Y., Park, J.-Y., Kim, J.-W., Lee, M.-J., Jang, M.-J., Lee, S.-Y., Baek, D.-W., Park, Y.-M., Lee, S.-W., Yoon, S., Bae, Y.-S., & Kwak, J.-Y. (2004). Differential expression of dendritic cell markers by all-trans retinoic acid on human acute promyelocytic leukemic cell line. International Immunopharmacology, 4(13), 1587-1601. https://doi.org/10.1016/j.intimp.2004.07.010
Park, S., Liu, M., & Kang, S. (2018). Alcohol Intake Interacts with CDKAL1, HHEX, and OAS3 Genetic Variants, Associated with the Risk of Type 2 Diabetes by Lowering Insulin Secretion in Korean Adults. Alcoholism, Clinical and Experimental Research, 42(12), 2326-2336. https://doi.org/10.1111/acer.13888
Park S H, Kang K, Giannopoulou E, Qiao Y, Kang K, Kim G, et al. Type I interferons and the cytokine TNF cooperatively reprogram the macrophage epigenome to promote inflammatory activation. Nat Immunol 2017; 18:1104-16. https://doi.org/10.1038/ni.3818
Peng, T., Thorn, K., Schroeder, T. et al. A BaSiC tool for background and shading correction of optical microscopy images. Nat Commun 8, 14836 (2017). https://doi.org/10.1038/ncomms14836
Pulit-Penaloza J A, Scherbik S V., Brinton M A. Type 1 IFN-independent activation of a subset of interferon stimulated genes in West Nile virus Eg101-infected mouse cells. Virology 2012; 425:82-94. https://doi.org/10.1016/j.virol.2012.01.006
Qing H, Sharma L, Hilliard B K, Peng X, Swaminathan A, Tian J, et al. Type I Interferon Limits Viral Dissemination-Driven Clinical Heterogeneity in a Native Murine Betacoronavirus Model of COVID-19. BioRxiv 2020:2020.09.11.294231. https://doi.org/10.1101/2020.09.11.294231
Schafer S L, Lin R, Moore P A, Hiscott J, Pitha P M. Regulation of Type I Interferon Gene Expression by Interferon Regulatory Factor-3. J Biol Chem 1998; 273:2714-20; Thanos D, Maniatis T. Virus induction of human IFN beta gene expression requires the assembly of an enhanceosome. Cell 1995; 83:1091-100. https://doi.org/10.1016/0092-8674(95)90136-1
Sheng, W., LaFleur, M. W., Nguyen, T. H., Chen, S., Chakravarthy, A., Conway, J. R., Li, Y., Chen, H., Yang, H., Hsu, P.-H., Van Allen, E. M., Freeman, G. J., De Carvalho, D. D., He, H. H., Sharpe, A. H., & Shi, Y. (2018). LSD1 Ablation Stimulates Anti-tumor Immunity and Enables Checkpoint Blockade. Cell, 174(3), 549-563.e19. https://doi.org/10.1016/j.cell.2018.05.052
Schoggins J W, Wilson S J, Panis M, Murphy M Y, Jones C T, Bieniasz P, et al. A diverse range of gene products are effectors of the type I interferon antiviral response. Nature 2011; 472:481-5. https://doi.org/10.1038/nature09907
Schulte-Schrepping J, Reusch N, Paclik D, Baßler K, Schlickeiser S, Zhang B, et al. Severe COVID-19 Is Marked by a Dysregulated Myeloid Cell Compartment. Cell 2020; 182:1419-1440.e23. https://doi.org/10.1016/j.cell.2020.08.001
Sirén J, Pirhonen J, Julkunen I, Matikainen S. IFN-α Regulates TLR-Dependent Gene Expression of IFN-α, IFN-β, IL-28, and IL-29. J Immunol 2005; 174:1932-7. https://doi.org/10.4049/jimmunol.174.4.1932
Thanos D, Maniatis T. Virus induction of human IFN beta gene expression requires the assembly of an enhanceosome. Cell 1995; 83:1091-100. https://doi.org/10.1016/0092-8674(95)90136-1
Thornbrough, J. M., Jha, B. K., Yount, B., Goldstein, S. A., Li, Y., Elliott, R., Sims, A. C., Baric, R. S., Silverman, R. H., & Weissa, S. R. (2016). Middle east respiratory syndrome coronavirus NS4b protein inhibits host RNase L activation. MBio, 7(2), 1-12. https://doi.org/10.1128/mBio.00258-16
Thummalapalli, R. (2018). EZH2 Inhibition Induces Endogenous Retroviral Elements and Primes Immunogenicity in Mesenchymal Small Cell Lung Cancer [Harvard]. http://nrs.harvard.edu/urn-3:HUL.InstRepos:37006471
Topper, M. J., Vaz, M., Chiappinelli, K. B., DeStefano Shields, C. E., Niknafs, N., Yen, R.-W. C., Wenzel, A., Hicks, J., Ballew, M., Stone, M., Tran, P. T., Zahnow, C. A., Hellmann, M. D., Anagnostou, V., Strissel, P. L., Strick, R., Velculescu, V. E., & Baylin, S. B. (2017). Epigenetic Therapy Ties MYC Depletion to Reversing Immune Evasion and Treating Lung Cancer. Cell, 171(6), 1284-1300.e21. https://doi.org/10.1016/j.cell.2017.10.022
Tseng C-T K, Perrone L A, Zhu H, Makino S, Peters C J. Severe Acute Respiratory Syndrome and the Innate Immune Responses: Modulation of Effector Cell Function without Productive Infection. J Immunol 2005; 174:7977 LP-7985. https://doi.org/10.4049/jimmunol.174.12.7977
VanLeuven J T, Ridenhour B J, Gonzalez A J, Miller C R, Miura T A. Lung epithelial cells have virus-specific and shared gene expression responses to infection by diverse respiratory viruses. PLoS One 2017; 12:e0178408-e0178408. https://doi.org/10.1371/journal.pone.0178408
Vianello P, Botrugno O A, Cappa A, Dal Zuffo R, Dessanti P, Mai A, et al. Discovery of a Novel Inhibitor of Histone Lysine-Specific Demethylase 1A (KDM1A/LSD1) as Orally Active Antitumor Agent. J Med Chem 2016; 59:1501-17. https://doi.org/10.1021/acs.jmedchem.5b01209
Wang, Y., Wang, Y., Chen, Y., & Qin, Q. (2020). Unique epidemiological and clinical features of the emerging 2019 novel coronavirus pneumonia (COVID-19) implicate special control measures. Journal of Medical Virology. https://doi.org/10.1002/jmv.25748
Wu, A., Peng, Y., Huang, B., Ding, X., Wang, X., Niu, P., Meng, J., Zhu, Z., Zhang, Z., Wang, J., Sheng, J., Quan, L., Xia, Z., Tan, W., Cheng, G., & Jiang, T. (2020). Genome Composition and Divergence of the Novel Coronavirus (2019-nCoV) Originating in China. Cell Host and Microbe, 1-4. https://doi.org/10.1016/j.chom.2020.02.001
Yang Z, Du J, Chen G, Zhao J, Yang X, Su L, et al. Coronavirus MHV-A59 infects the lung and causes severe pneumonia in C57BL/6 mice. Virol Sin 2014; 29:393-402. https://doi.org/10.1007/s12250-014-3530-y
Ye, Y., Hauns, K., Langland, J. O., Jacobs, B. L., & Hogue, B. G. (2007). Mouse hepatitis coronavirus A59 nucleocapsid protein is a type I interferon antagonist. Journal of Virology, 81(6), 2554-2563. https://doi.org/10.1128/JVI.01634-06
Yilla M, Harcourt B H, Hickman C J, McGrew M, Tamin A, Goldsmith C S, et al. SARS-coronavirus replication in human peripheral monocytes/macrophages. Virus Res 2005; 107:93-101. https://doi.org/10.1016/j.virusres.2004.09.004
Zhang L, Richards A, Khalil A, Wogram E, Ma H, Young R A, et al. SARS-CoV-2 RNA reverse-transcribed and integrated into the human genome. BioRxiv Prepr Serv Biol 2020:2020.12.12.422516. https://doi.org/10.1101/2020.12.12.422516
Zhao, J., Wohlford-Lenane, C., Zhao, J., Fleming, E., Lane, T. E., McCray, P. B. J., & Perlman, S. (2012). Intranasal treatment with poly(I*C) protects aged mice from lethal respiratory virus infections. Journal of Virology, 86(21), 11416-11424. https://doi.org/10.1128/JVI.01410-12
Zhou H, Zhao J, Perlman S. Autocrine interferon priming in macrophages but not dendritic cells results in enhanced cytokine and chemokine production after coronavirus infection. MBio 2010; 1:1-9. https://doi.org/10.1128/mBio.00219-10
Zhou H, Perlman S. Mouse Hepatitis Virus Does Not Induce Beta Interferon Synthesis and Does Not Inhibit Its Induction by Double-Stranded RNA. J Virol 2007; 81:568-74. https://doi.org/10.1128/jvi.01512-06
Zhou Z, Ren L, Zhang L, Zhong J, Xiao Y, Jia Z, et al. Heightened Innate Immune Responses in the Respiratory Tract of COVID-19 Patients. Cell Host Microbe 2020; 27:883-890.e2. https://doi.org/10.1016/j.chom.2020.04.017

1. A method for the treatment and/or prevention of a viral infection and/or viral disease caused by and/or associated with RNA viruses, comprising administering an LSD1 inhibitor to a patient in need thereof.

2. The method according to claim 1 wherein the RNA viruses are Coronaviridae.

3. The method of claim 1, wherein said LSD1 inhibitor is selected from:
a) a compound of general formula (I) 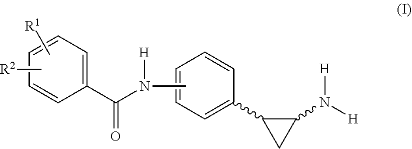
or a pharmaceutically acceptable salt or solvate thereof, wherein
R¹ is 4-methylpiperazin-1-yl, 1-methylpiperidin-4-yl, 2-oxooxazolidin-3-yl, piperidin-1-yl or morpholin-4-yl; and
R² is hydrogen, halogen, C₁₋₆ alkyl, C₁₋₆ alkoxy, C₁₋₆ haloalkyl, C₁₋₆ haloalkoxy, or benzyloxycarbonylamino;
or
b) a compound selected from the group consisting of:
ORY-1001 (or iadademstat), CC-90011, ORY-2001 (or vafidemstat), GSK-2879552, IMG-7289 (or bomedemstat), INCB059872, 4SC-202 (or domatinostat), Seclidemstat, TAK-418, SYHA-1807, BEA-17, HM-97211, HM-97346, JBI-097, JBI-128, ORY-3001, RN-1, SP-2509, T-3775440, T-448, EPI-110; and pharmaceutically acceptable salt or solvate thereof.

4. The method according to claim 3 wherein in the general formula (I):
R¹ is 4-methylpiperazin-1-yl and R² is hydrogen; or
R¹ is 4-methylpiperazin-1-yl and R² is benzyloxycarbonylamino; or
R¹ is 1-methylpiperidin-4-yl and R² is hydrogen; or
R¹ is 1-methylpiperidin-4-yl and R² is benzyloxycarbonylamino; or
R¹ is 2-oxooxazolidin-3-yl and R² is hydrogen; or
R¹ is 2-oxooxazolidin-3-yl and R² is benzyloxycarbonylamino;
R¹ is piperidin-1-yl and R² is hydrogen; or
R¹ is piperidin-1-yl and R² is benzyloxycarbonylamino; or
R¹ is morpholin-4-yl and R² is hydrogen; or
R¹ is morpholin-4-yl and R² is benzyloxycarbonylamino; or
and pharmaceutically acceptable salt or solvate thereof.

5. The method of claim 4, wherein the LSD1 inhibitor is selected from:
N-[4-[(1S,2R)-2-aminocyclopropyl]phenyl]-4-(4-methylpiperazin-1-yl)benzamide;
N-[4-[(1S,2R)-2-aminocyclopropyl]phenyl]-3-(2-oxooxazolidin-3-yl)benzamide;
N-[4-[(1R,2S)-2-aminocyclopropyl]phenyl]-4-(4-methylpiperazin-1-yl)benzamide;
N-[4-[(1R,2S)-2-aminocyclopropyl]phenyl]-3-(2-oxooxazolidin-3-yl)benzamide;
N-[4-[trans-2-aminocyclopropyl]phenyl]-4-(4-methylpiperazin-1-yl)benzamide;
N-[4-[trans-2-aminocyclopropyl]phenyl]-4-(1-methylpiperidin-4-yl)benzamide;
N-[4-[trans-2-aminocyclopropyl]phenyl]-3-(2-oxooxazolidin-3-yl)benzamide;
N-[4-[trans-2-aminocyclopropyl]phenyl]-4-(2-oxooxazolidin-3-yl)benzamide;
Benzyl N-[5-[[4-(trans-2-aminocyclopropyl)phenyl]carbamoyl]2-(4-methylpiperazin-1-yl)phenyl]carbamate;
Benzyl N-[4-[[4-(trans-2-aminocyclopropyl)phenyl]carbamoyl]2-(4-methylpiperazin-l-yl)phenyl]carbamate;
Benzyl N-[5-[[4-trans-2-aminocyclopropyl]phenyl]carbamoyl]-2-(1-piperidyl)phenyl]carbamate;
Benzyl N-[5-[[4-[trans-2-aminocyclopropyl]phenyl]carbamoyl]-2-morpholinophenyl] carbamate;
and pharmaceutically acceptable salt or solvate thereof.

6. The method of claim 1, wherein the LSD1 inhibitor is selected from the group consisting of: N-[4-[(1S,2R)-2-aminocyclopropyl]phenyl]-4-(4-methylpiperazin-1-yl)benzamide, N-[4-[trans-2-aminocyclopropyl]-phenyl]-4-(4-methylpiperazin-1-yl)benz amide, N-[4-[(1R,2S)-2-aminocyclopropyl] phenyl]-4-(4-methylpiperazin-1-yl)benzamide, ORY-1001 (or iadademstat), ORY-2001 (or vafidemstat) and a pharmaceutically acceptable salt or solvate thereof.

7. The method of claim 1 wherein the inhibitor inhibits and/or prevents the viral induced increased expression of inflammatory cytokines while sparing the expression of Interferon and Interferon-Stimulated Genes, wherein the inflammatory cytokines comprise Ccl3, Ccl4, Ccl5, Ccl8, Cxcl2, Cxcl3, Il10, Il12a, Il12b, Il1a, Il1b, 116, TNFa and their orthologs in different species and the Interferon and Interferon-Stimulated genes comprise Ifit1, Ifit2, Ifit3, Ifitm3, Isg15, Mx1, Mx2, Oas1, Oas2, Oas3, MDA5, RIG-I, Irf1, Irf2, Irf7 and genes encoding for type I, II and III interferon and their orthologs in different species.

8. The method of claim 1, further comprising administering at least one other therapeutic agent.

9. (canceled)

10. The method of claim 1, wherein the viral infection is an infection of the respiratory tract and/or of the gastrointestinal tract and/or kidneys and/or central nervous system.

11. The method of claim 1, wherein the viral infection and/or disease is a Coronavirus infection and/or disease.

12. The method of claim 11, wherein the Coronavirus infection and/or disease is selected from the group consisting of: COVID-19, SARS, MERS, any other severe acute respiratory syndrome caused by Coronavirus, upper respiratory tract infections, pneumonia, pneumonitis, and bronchitis.
